(19) United States
(12) Patent Application Publication
(10) Pub. No.: US 2020/0155611 A1
(43) Pub. Date: May 21, 2020

(54) METHODS AND COMPOSITIONS FOR TREATING ADDICTIONS

(71) Applicants: PLURISTEM LTD., Haifa (IL); BAR ILAN UNIVERSITY, Ramat Gan (IL)

(72) Inventors: Gal YADID, Shoham (IL); Rachel OFIR, Adi (IL); Niva SHRAGA HELED, Nahariya (IL)

(73) Assignees: PLURISTEM LTD., Haifa (IL); BAR ILAN UNIVERSITY, Ramat Gan (IL)

(21) Appl. No.: 16/631,861

(22) PCT Filed: Jul. 23, 2018
(86) PCT No.: PCT/IB2018/055473

§ 371 Date: Jan. 17, 2020

Related U.S. Application Data
(60) Provisional application No. 62/535,899, filed on Jul. 23, 2017.

(51) Int. Cl. A61K 35/35; A61K 9/00; A61P 25/30

(52) U.S. Cl. CPC A61K 35/35; A61P 25/30; A61K 9/0085; A61K 9/0043

(57) ABSTRACT

Disclosed herein are methods and compositions comprising adherent stromal cells.

### CROSS-REFERENCE TO RELATED APPLICATIONS

This application claims priority to U.S. Application No. 62/535,899, filed Jul. 23, 2017, which is hereby incorporated by reference in its entirety for all purposes

### FIELD

Disclosed herein are methods and compositions comprising adherent stromal cells.

### BACKGROUND

Craving and relapse is a major problem in treatment of addiction to cocaine and other substances. In humans, craving and relapse can be triggered by acute exposure to the drug, drug-associated cues, or stress. Cue-induced craving does not decay, but progressively intensifies, or ‘incubates’, over the first few weeks of withdrawal, and persists over extended periods of time. As a result, addicts are prone to relapse even after long durations of abstinence. Recent evidence in humans has shown time-dependent increases in cue-induced nicotine, alcohol, and methamphetamine craving during abstinence. An analogous phenomenon of incubation of cocaine craving has been identified in rats.

Currently, there are no FDA-approved medications for safe and effective treatment of cocaine addiction, and existing treatments do not show great efficacy in longitudinal studies where long-term abstinence is the primary outcome of interest. Several phase II clinical trials are currently examining the efficacy of drugs that modulate dopamine, serotonin, noradrenaline, GABA, or glutamate signaling. However, abusers' tendency not to complete treatment programs, and their propensity for relapse, further complicates treatment efficacy. Methods and compositions for treating addiction are urgently needed in the art.

### SUMMARY

In one embodiment, there is provided a method of treating an addiction in a subject in need thereof, comprising the step of administering to the subject a pharmaceutical composition comprising adherent stromal cells (ASC), thereby treating an addiction. In certain embodiments, the ASC are derived from a placenta. In other embodiments, the ASC are not derived from placental tissue, and may be e.g. derived from adipose tissue, or from bone marrow (BM).

In another embodiment, there is provided a pharmaceutical composition for treating an addiction, comprising a therapeutically effective amount of ASC. In certain embodiments, the ASC are derived from a placenta. In other embodiments, the cells are from adipose tissue, or BM. In other embodiments, the ASC are derived from another tissue source.

In another embodiment, there is provided use of ASC in the preparation of a medicament for treating addiction. In certain embodiments, the ASC are derived from a placenta. In other embodiments, the cells are derived from adipose tissue, or BM, or from another tissue.

In certain embodiments, the ASC described herein have been cultured on 2-dimensional (2D) substrate, a 3-dimensional (3D) substrate, or a combination thereof. Non-limiting examples of 2D and 3D culture conditions are provided in the Detailed Description and in the Examples.

Reference herein to “growth” of a population of cells is intended to be synonymous with expansion of a cell population.

Except where otherwise indicated, all ranges mentioned herein are inclusive.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the embodiments of the invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a diagram of a bioreactor that can be used to prepare the cells.

FIG. 2 contains pictures of bone marrow (BM)-derived MSC (top row) or placental cells after adipogenesis assays. Cells were incubated with (left column) or without (right column) differentiation medium. Placental ASC were expanded in SRM (middle 3 rows depict 3 different batches) or in full DMEM (bottom row).

FIG. 3 contains pictures of BM-derived MSC (top row) or placental cells after osteogenesis assays. Cells were incubated with (left column) or without (right column) differentiation medium. Placental ASC were expanded in SRM (middle 3 rows depict 3 different batches) or in full DMEM (bottom row).

FIG. 4 is a table showing various combinations and concentrations of factors tested for their effects on BDNF secretion, and the test results.

FIG. 5 is a plot showing BDNF concentration in CM collected from cells following incubation under various conditions, after seeding at 0.4, 0.8, or 2.9×10\hat{ }6 cells and growth for 5 days (left, middle, and right set of bars, respectively). For the left and middle sets, the left, middle and right bar within each set depicts incubation with no induction agents, or with induction agents for the last 24 hr. or the last 72 hr., respectively. For the right set of bars, the left bar depicts no induction agents, and right bar depicts a 72-hr incubation with induction agents. Cells seeded at 0.4 and 0.8×10\hat{ }6 cells/flask were grown for the whole period in DMEM+20% FBS, whereas cells seeded at 2.9×10\hat{ }6 cells/flask were grown in basal DMEM supplemented with glutamine and antibiotics for the last 72 hr. Vertical axis: BDNF concentration in pg per 10⁶ cells.

FIGS. 6A-D are plots showing BDNF concentration in CM collected from cells, after seeding (as described for FIG. 5) at 0.4, 0.8, or 2.9×10\hat{ }6 cells (A, B, and C, respectively), grown with or without serum supplementation which were or were not induced for 24 or 72 hours, following which the cells were cryopreserved, thawed and seeded equally in 6-well plates for 72 hours (0.5*10⁶ cells/well), and medium was sampled after 24, 48, and 72 (left, middle, and right bar, respectively, in each series). Cells seeded at 0.4 and 0.8×10\hat{ }6 cells/flask were grown for the whole period in DMEM+20% FBS, whereas cells seeded at 2.9×10\hat{ }6 cells/flask were grown in basal DMEM supplemented with glutamine and antibiotics for the last 72 hr. Different sets of bars depict different induction conditions (before cryopreservation). Namely, for A-B, the left, middle, and right sets of bars depict incubation with no induction agents, or with induction agents for the last 24 hr. or the last 72 hr., respectively. For C, the left and right sets of bars depict incubation with no induction agents or with induction agents for the last 72 hr., respectively. D depicts BDNF data from the 72-hr. timepoint of the experiment described above, but normalized to the number of cells that were harvested 72 hr after cell thawing, Vertical axis: BDNF concentration in pg/ml (A-C) or pg per 10⁶ cells (D).

FIGS. 7A-C are plots showing concentrations of very highly-, highly-, and medium-expressed cytokines (A, B, and C, respectively) in CM collected from ASC that were either induced for 24 h or not. Following induction (or not) cells were cryopreserved. After thawing, cells were seeded 0.5*10\hat{ }6 cells in 6 well plates in DMEM with 10% FBS, Glutamine and antibiotics. After 24 hours, cells were washed and resuspended in DMEM w/o FBS for an additional 24 h. Horizontal axis indicates the measured cytokines in the medium collected after the last incubation, for each set of bars. For the each set of bars, the left and right bars indicate incubation with no induction agents or 24-hr incubation with induction agents, respectively. Vertical axis: cytokine concentration in pg/10\hat{ }6 cells.

FIG. 8A contains microscopy images depicting staining of undifferentiated neurons (negative control; upper left panel), or neurons differentiated with 1 mM cAMP (upper middle panel), 10 mcM butyric acid (upper right panel), or CM from ASC incubated without induction agents (lower panels). Cells are stained for human βIII-tubulin (red) and human tyrosine hydroxylase (green). Nuclei are stained with DAPI (seen in blue). B depicts the relative percentage of differentiated neurons (vertical axis) in SH-SY5Y cells untreated or exposed to butyric acid, cAMP, or ASC-derived CM respectively (bars ordered left to right). The depicted percentages are the averages of 4 different batches. C is a plot of viability of differentiated SH-SY5Y cells. Cells were pretreated with either control medium (white circles), or CM from placental ASC subjected to either bioreactor expansion (black circles, solid line) or incubation with bFGF, N-2 supplement, heparin and cAMP (black circles, dotted line) Two hours later, H₂O₂ (200 μM) was added to each medium. Luminescence (vertical axis) was recorded every 15 minutes for 8 hours and is expressed as percent of control, which is the cells exposed to same medium without H₂O₂; horizontal axis shows time (hours) after the addition of H₂O₂. D. Column chart at the 6.5-hour timepoint for the experiment described for C. Black, white, and gray bars show control medium, bioreactor-expanded, and induction agent groups, respectively. E. Plot showing level of reactive oxygen species (ROS) in differentiated SH-SY5Y cells, after treatment with H₂O₂ (200 μM) in either control medium (gray circles), or CM from placental ASC subjected to either bioreactor expansion (black circles, solid line) or incubation with bFGF and cAMP (black circles, dotted line). Fluorescence (vertical axis) was recorded every 15 minutes for 6 hours and is expressed as fold change relative to control, which is the cells exposed to same medium without H₂O₂; horizontal axis shows time (hours) after the addition of H₂O₂.

FIG. 9A is a chart of liver (black bars) and lung (white bars) distribution of GNP-stained placental ASC in rats after i.v. (left) or i.n. (right) administration. N=2. Standard error of the mean (SEM) is <5%. Abbreviations: # (number of MSCs); IN (intranasal); IV (intravenous); Li (liver); Lu (lung). B—C are CT images of GNP-stained placental ASC in rat brains. Gold nanoparticle (GNP)-stained ASCs are seen as green dots. Depicted are CT images including the brain area of intranasally injected rats (B) and TV-injected rats (C). Coronal (left) and sagittal (right) views are shown for (B); coronal view is shown for (C). D is a graph showing ASC concentration (by FAAS analysis) in prefrontal cortex (PFC), nucleus accumbens (NAc), striatum (ST), dentate gyrus (DG), central amygdala, ventral tegmental area (VTA), ventricles, cerebellum, and brain stem, detected 28 days after i.n. or i.c.v. administration of GNP-stained placental ASC (first and second bar in each series, respectively).

FIG. 10A is a graph showing distribution of rat BM-MSC in rats given MSC intranasally. Other abbreviations: #/W (number of MSCs per dry weight (mg) of tissue); C (cerebellum); BS (brain stem); R (rest of brain). B—C are fluorescent images of DG showing MSCs labeled by fluorescent dye [(1,1′-dioctadecyl-3,3,3′,3′-tetramethylindocarbocyanine perchlorate [DiI]; from Sigma) (red) and DAPI (Blue)] (B) and hippocampal neuronal proliferation (magnification×200; C) 1 month after injection into the left ventricle. At 1 month after MSC administration, frozen sections of the DG area of the hippocampus (HC) were analyzed for MSC engraftment (using DiI labeling) and neurogenesis. Proliferating cells were detected by immunofluorescent labeling of proliferating cell nuclear antigen (PCNA). In C, PCNA-positive cells in the ipsilateral hippocampus (left) and contralateral hippocampus (right) are indicated by arrows.

FIG. 11A is a graph of number of active (cocaine-supplying) lever presses per hour by rats subjected to cocaine self-administration training sessions for one hour each day (1.5 mg/kg, 10 days); followed by administration of vehicle (circles; n=3) or GNP-stained ASC intracerebroventricularly (i.c.v.) (triangles; n=7) or intranasally (i.n) (squares; n=7) (n=3) on day 10, followed by extinction sessions (days 11-19) in which the rats are placed back in a cage where the active level no longer supplies cocaine. B—C are graphs of the number of active lever presses after cocaine-primed reinstatement (10 mg/kg cocaine, i.p.) 2 weeks (B) or four weeks (C) after cell administration, in rats given vehicle (left bar) or ASC i.c.v. (middle bar) or i.n (right bar). Values are presented as mean+SEM. Horizontal axis indicates study day numbers, which were assigned only on days when animals received training sessions.

FIGS. 12A-B are graphs showing presses of active (cocaine-supplying) and inactive levers (per hour; vertical axis) in rats addicted to cocaine and administered maternal (A) or fetal (B) ASC at the beginning of the extinction period. N=6, 9, and 8 in placebo, maternal, and fetal groups. Horizontal axis indicates study day numbers, which were assigned only on days when animals received training sessions. Values are presented as mean+SEM. C-D are bar graphs showing active lever presses during the first day of extinction (C) and induction of relapse (D). For C, bars from left show addicted/untreated, fetal-treated, maternal-treated, and sham groups. For D, bars from left show addicted/untreated, maternal-treated, fetal-treated, and sham groups.

FIG. 13 is a graph showing BDNF mRNA expression levels relative to control (vertical axis) in the DG in ASC-treated rats.

FIGS. 14A-B are graphs showing relative expression levels (vertical axis) of miR484 (A) and miR124 (B) in DG in placental ASC-treated rats, after removal of brains from animals (performed after the relapse session −12 days after ASC administration) C shows correlation of miR484 levels in DG with craving levels (P=0.0119; r=0.7230). D shows correlation of miR484 in the DG and miR124 levels in the NAc in maternal ASC-treated rats (P=0.0050, r=0.9419).

FIGS. 15A-B are graphs showing relative expression levels (vertical axis) of miR484 (A) and miR124 (B) in NAc in placental ASC-treated rats after the relapse session. C-D show correlation of miR484 (C) and miR124 (D) levels in the NAc with craving levels. For C, P=0.0006, and r=−0.9592. For D, P=0.0171, and r=−0.8434

FIG. 16 shows miRNA (miR) expression in VTA of addicted rats (“treated rats”) compared to sham-operated (“untreated”) rats. A is a scatter plot depicting the expression profile of 423 miRNAs obtained by Nanostring nCounter analysis system. Each dot represents the fold-change in the expression of a certain miRNA in treated vs. untreated rats. B shows 12 miRNAs out of the 423 miRNA presented in A, which showed a statistically significant change (p<0.05) in expression levels in treated vs. untreated rats (n=6). In A-B, positive and negative values represent an increase or decrease (respectively) in the expression level in treated vs. untreated rats. Abbreviations: FC (fold-change); 484 (rno-miR-484); 7i (rno-let-7i); 3583 (rno-miR-3583-3p); 30a (rno-miR-30a); 490 (rno-miR-490); 451 (rno-miR-451); 139 (rno-miR-139-3p); 103 (rno-miR-103); 378 (rno-miR-378); 16 (rno-miR-16); 21 (rno-miR-16); 325 (rno-miR-325-5p).

### DETAILED DESCRIPTION

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Aspects of the invention relate to methods and compositions that comprise adherent stromal cells (ASC). In some embodiments, the ASC are derived from placenta. In other embodiments, the ASC are derived from non-placental tissue, which may be e.g. adipose tissue, bone marrow (BM), or other tissues. Alternatively or in addition, the ASC may be human ASC, or in other embodiments animal ASC.

In some embodiments, there is provided a method of treating an addiction in a subject in need thereof, comprising the step of administering to the subject a pharmaceutical composition comprising ASC, thereby treating an addiction.

In other embodiments, there is provided a method of inhibiting relapse of an addiction in a subject in need thereof, comprising the step of administering to the subject a pharmaceutical composition comprising ASC, thereby inhibiting relapse of an addiction.

In other embodiments, there is provided a method of treating a substance addiction in a subject in need thereof, comprising the step of administering to the subject a pharmaceutical composition comprising ASC, thereby treating substance addiction.

In other embodiments, there is provided a method of reducing a substance craving in a subject in need thereof, comprising the step of administering to the subject a pharmaceutical composition comprising ASC, thereby reducing substance craving.

Also provided herein is a method of treating an addiction in a subject in need thereof, comprising: administering to the subject a pharmaceutical composition, comprising placental ASC, thereby treating an addiction. In various embodiments, the ASC may be maternal, fetal, or a mixture thereof.

Provided herein, in other embodiments, is a method of treating an addiction in a subject in need thereof, comprising: administering to the subject a pharmaceutical composition, comprising ASC not derived from placental tissue, thereby treating an addiction. In various embodiments, the ASC may be derived from adipose tissue, BM, or another tissue, e.g. a tissue mentioned herein.

Also provided herein are placental ASCs for use in a method of treating addiction, said method comprising administering a pharmaceutical composition, comprising placental ASC. In various embodiments, the ASC may be maternal, fetal, or a mixture thereof.

Also provided herein are non-placental ASCs for use in a method of treating addiction, said method comprising administering a pharmaceutical composition, comprising non-placental ASC. In various embodiments, the ASC may be derived from adipose tissue, BM, or another tissue, e.g. a tissue mentioned herein.

Also provided herein are allogeneic placental ASCs for use in a method of treating addiction, said method comprising the steps of: (a) administering a first pharmaceutical composition, comprising allogeneic placental ASC from a first donor; and subsequently (b) administering a second pharmaceutical composition comprising allogeneic placental ASC from a second donor, wherein the second donor differs from the first donor in at least one allele group of HLA-A or HLA-B; wherein the administrations are separated in time from each other by at least 7 days; and wherein the ASC secrete a neurotrophic and/or neuroprotective factor. In various embodiments, the ASC may be maternal, fetal, or a mixture thereof.

Also provided herein are allogeneic non-placental ASCs for use in a method of treating addiction, said method comprising the steps of: (a) administering a first pharmaceutical composition, comprising allogeneic ASC from a first donor; and subsequently (b) administering a second pharmaceutical composition comprising allogeneic ASC from a second donor, wherein the second donor differs from the first donor in at least one allele group of HLA-A or HLA-B; wherein the administrations are separated in time from each other by at least 7 days; and wherein the ASC secrete a neurotrophic and/or neuroprotective factor. In various embodiments, the ASC may be derived from adipose tissue, BM, or another tissue, e.g. a tissue mentioned herein.

In certain embodiments, the described first ASC population and second ASC population are derived from the same tissue, which may be, in some embodiments, adipose tissue, or, in other embodiments, bone marrow. In still other embodiments, the tissue is another source of ASC.

Allogeneic, as used herein (except where indicated otherwise), refers to a biological material (e.g. ASC) not derived from, and not syngeneic with, the subject being treated. Typically, allogeneic ASC are neither syngeneic nor haploidentical with the subject.

In certain embodiments, the described allogeneic ASC from the first donor and the second donor (also referred to herein as “first ASC population” and “second ASC population”, respectively) are derived from the same tissue, which may be, in some embodiments, placenta. In other embodiments, the tissue is adipose, or is bone marrow. In still other embodiments, the tissue is another source of ASC.

Alternatively or in addition, the first ASC population and second ASC population exhibit common characteristics. In some embodiments, the common characteristics relate to the cells' therapeutic potential. Certain embodiments of such common characteristics are described herein. In other embodiments, the common characteristic is selected from population doubling time (PDL; this parameter may be derived from population doubling level) and glucose consumption rate (GCR), or in other embodiments is a combination thereof. In certain embodiments, the PDL and/or GCR are measured in bioreactor culture in 3D fibrous carriers, e.g. as described herein in Example 4, following cell expansion as described in Example 1, or in other embodiments, in Examples 2-3. In certain embodiments, the 2 populations are within 2 fold of each other for GCR on day 5 of bioreactor culture. In other embodiments, the GCR is measured on day 3, day 4, or day 6. Alternatively or in addition, the 2 populations are within 1.5 fold, within 3 fold, or within 5 fold of each other for the specified parameter.

Reference to ASC “from” or “derived from” a donor is intended to encompass cells removed from or otherwise obtained from the donor, followed by optional steps of ex-vivo cell culture, expansion, and/or other treatments to improve the therapeutic efficacy of the cells; and/or combination with pharmaceutical excipients. Those skilled in the art will appreciate that the aforementioned optional steps will not alter the HLA genotype of the ASC, absent intentional modification of the HLA genotype (e.g. using CRISPR-mediating editing or the like). Cell populations with an intentionally modified HLA genotype are not intended to be encompassed. ASC populations that contain a mixture cells from more than one donor are also not intended to be encompassed.

As will be appreciated by those skilled in the art, the HLA system or complex is a gene complex encoding the major histocompatibility complex (MHC) proteins in humans. These cell-surface proteins are involved in regulation of the immune system in humans. The HLA gene complex resides on a 3-Mbp stretch within chromosome 6p21. HLA genes are highly polymorphic. HLAs encoding MHC class I proteins (“class I HLA's”) present peptides from inside the cell, while class H HLA's present external peptides.

There are 3 major MHC class I genes, HLA-A, HLA-B, and HLA-C; and 3 minor class I genes, HLA-E, HLA-F and HLA-G. β2-microglobulin binds with major and minor gene subunits to produce a heterodimer.

There are 3 major (DP, DQ and DR) and 2 minor (DM and DO) MHC class II proteins encoded by the HLA. The class II MHC proteins combine to form heterodimeric (αβ) protein receptors that are typically expressed on the surface of antigen-presenting cells.

HLA alleles are often named according to a multi-partite system, where the letter prefix (e.g. “HLA-A”) denotes the locus, followed by an asterisk, followed by the “allele group” number, followed by the specific HLA protein number, followed by a number used to denote silent DNA mutations in a coding region, followed by, lastly, a number used to denote DNA mutations in a non-coding region (Robinson J et al). Typically, the allele group corresponds to the encoded serological antigen, while specific HLA proteins within an allele group exhibit relatively minor antigenic differences. For example, in the hypothetical allele “HLA-A*02:07:01:03”, the allele group number is 02; 07 is the specific HLA protein number; 01 describes a pattern of silent DNA mutations in the coding regions; and 03 describes a pattern of DNA mutations in non-coding regions. “Mutations” in this regard refers to variations relative to the founder (initially identified) allele in the allele group.

HLA typing at each locus, may be, in some embodiments, low resolution, or “first-level field” typing, by reference to the two digits preceding the first separator, or antigen level typing, e.g. A*02 in the above example. In various other embodiments, the typing is at “intermediate-level” resolution, i.e. second-level field, e.g. HLA-A*02:07, or in other embodiments, third-level field, e.g. HLA-A*02:07:01. In other embodiments, the typing is “allele level typing”, using all digits in the first, second, third and fourth fields, e.g. HLA-A*02:07:01:03.

Allele groups are clustered into “supertypes” which have similar peptide binding repertoires. Examples of HLA-A supertypes are 1, 2, 3, and 24, and examples of HLA-B supertypes are 7, 27, 44, 58, and 62.

Reference to a second donor “differ/differs/differing” from a first donor in at least one allele group of HLA-A or HLA-B denotes that the DNA of the second donor comprises at least one HLA-A or HLA-B allele belonging to an allele group not represented in the alleles of the first donor. (Typically [except in the case of a homozygous first donor], the DNA of the first donor will also comprise at least one HLA-A or HLA-B allele belonging to an allele group not represented in the alleles of the second donor). Similarly, a second donor “differs from” a first donor in at least one allele supertype if the DNA of the second donor comprises at least one HLA-A or HLA-B allele belonging to a supertype not represented in the alleles of the first donor. These terms are intended to be used analogously in various contexts herein, except where indicated otherwise.

In other embodiments, the second donor in the described therapeutic methods and compositions differs from the first donor in at least one allele group of HLA-A. In still other embodiments, the second donor differs from the first donor in at least one allele group of HLA-B.

In yet other embodiments, the second donor differs from the first donor in at least two HLA-A allele groups of or, in other embodiments, in at least 2 HLA-B allele groups; or, in other embodiments, at least one allele group of each of HLA-A and HLA-B.

In other embodiments, the second donor differs from the first donor in at least one HLA-A allele supertype or, in other embodiments, at least one HLA-B allele supertype.

In still other embodiments, the second donor differs from the first donor in at least two allele supertypes of HLA-A or HLA-B, which may be, in more specific embodiments, an HLA-A allele supertype, an HLA-B allele supertype, or a combination thereof.

Alternatively or in addition, the second donor differs from the first donor in at least one allele group of HLA-DR, or in other embodiments, in 2 HLA-DR allele groups.

Step b) of the described method (administering a second pharmaceutical composition comprising allogeneic ASC from a second donor) is, in various embodiments, performed between 2-52 weeks. In other embodiments, step b) is performed between 3-52, 4-26, 5-26, 6-20, 6-18, 6-15, 6-10, 3-20, 3-15, 3-10, 4-12, 4-20, 5-18, 6-16, 8-16, 10-16, or 8-12 weeks after step a).

Alternatively or in addition, step b) of the described methods is followed by an additional step, comprising the step of administering to the subject, at least 7 days after step b), a third pharmaceutical composition comprising allogeneic ASC derived from a third donor, wherein the third donor differs from both the first donor and the second donor in at least one allele group of HLA-A or HLA-B (i.e. has an allele group not represented in either the first or second donor), which is, in various embodiments, an allele of HLA-A or HLA-B. In other embodiments, the third donor differs from both the first donor and the second donor in at least two allele groups of HLA-A or HLA-B, which are, in various embodiments, an allele of HLA-A, HLA-B, or a combination thereof.

The aforementioned addiction, is, in some embodiments, an addiction to a psychostimulant. In certain embodiments, the psychostimulant is a sympathomimetic, i.e. a substance that stimulates the sympathetic branch of the autonomic nervous system. In other embodiments, the psychostimulant is a Dopamine Reuptake Inhibitor (DRI), non-limiting embodiments of which include cocaine, methamphetamine, amphetamine, and methylphenidate. In still other embodiments, the psychostimulant is selected from the group consisting of methylenedioxymethamphetamine (“ecstasy”), N-methylamphetamine, crystal N-methylamphetamine, phenylethylamine, dexamphetamine, phentermine, paramethoxyamphetamine, cocaine, and crack cocaine.

In other embodiments, the addiction is to a depressant. Non-limiting examples of depressants are opiates, opioids, alcohol, barbiturates, tranquilizers and benzodiazepines. In other embodiments, the depressant is selected from cannabis, GHB, heroin, morphine, codeine, methadone, buprenorphine, pethidine, dilaudid, and kapanol.

In still other embodiments, the addiction is to a hallucinogen. In other embodiments, the addiction is to a psychedelic compound, non-limiting examples of which are dimethyltryptamine (DMT), Ayahuasca, Psilocybin, Mescaline, and D-lysergic acid diethylamide (LSD). In still other embodiments, the addiction is to a dissociative compound, non-limiting examples of which are ketamine, methoxetamine (MXE), phencyclidine (PCP), and dextromethorphan (DXM). In yet other embodiments, the addiction is to a deliriant compound, non-limiting examples of which are [Atropa belladonna (deadly nightshade), Brugmansia species (Angel's Trumpet), Datura stramonium (Jimson weed), Hyoscyamus niger (henbane), Mandragora officinarum (mandrake), and Myristica fragrans (nutmeg). In other embodiments, the hallucinogen is selected from LSD, psilocybin, PCP (phencyclidine), ketamine, and mescaline.

Methods for assessing addiction are known in the art and are described herein (Examples), Additional non-limiting methods are described, for example, in Waters R P I et al. Such methods utilize, in some embodiments, laboratory animals (e.g. rats) to model cocaine addiction by examining criteria of addiction-like behaviors, such as persistent seeking in the absence of drug, high motivation for drug, and/or resistance to punishment during drug seeking, to detect subjects that possess an addiction phenotype. Other methods may utilize using cerebral metabolic rate of oxygen (CMRO₂) as a surrogate marker of aggregated neural activity, e.g. in human subjects (Liu P et al). Still other methods comprising measuring micro-RNA's (miRNA's) involved in addiction. Non-limiting examples of such miRNA's are miRNA-124 and miRNA-451, miRNA488. In still other embodiments, miRNA levels are measured in the brain, for example in neural regions of interest, e.g. the dentate gyrus (DG) and/or the Nucleus Accumbens.

Methods for measuring neuro-inflammation are known to those skilled in the art, and include, for example, staining of microglia (Das M et al) and various other methods described in Xiong Y et al, Nizamutdinov D et al, Hartl R et al, and the references cited therein.

In various embodiments, the ASC are administered to the subject within 1 hour, within 2 hours, within 3 hours, within 4 hours, within 6 hours, within 8 hours, within 10 hours, within 12 hours, within 15 hours, within 18 hours, within 24 hours, within 30 hours, within 36 hours, within 48 hours, within 3 days, within 4 days, within 5 days, within 6 days, within 8 days, within 10 days, within 12 days, or within 20 days of the last exposure to the addicting substance. In more specific embodiments, the described compositions are administered 1-24, 2-24, 3-24, 4-24, 5-24, 6-24, 8-24, 10-24, 12-48, 1-48, 2-48, 3-48, 4-48, 5-48, 6-48, 8-48, 10-48, 12-48, 18-48, 24-48, 1-72, 2-72, 3-72, 4-72, 5-72, 6-72, 8-72, 10-72, 12-72, 18-72, 24-72, or 36-72 hours after the last exposure to the addicting substance. In still other embodiments, the described compositions are administered 3-48, 4-48, 5-48, or 6-48 hours after the last exposure to the addicting substance.

In various embodiments, the described cells are able to exert the described therapeutic effects, each of which is considered a separate embodiment, with or without the cells themselves engrafting in the host. For example, the cells may, in various embodiments, be able to exert a therapeutic effect, without themselves surviving for more than 3 days, more than 4 days, more than 5 days, more than 6 days, more than 7 days, more than 8 days, more than 9 days, more than 10 days, more than 14 days, more than 20 days, more than 30 days, more than 40 days, more than 50 days, or more than 60 days after administration.

Those skilled in the art will appreciate that cocaine increases neuroinflammation in the nucleus accumbens (NAc) and the ventral tegmental area (VTA), (Vallender E J et al). In certain embodiments, treatment with the described ASC results in reduced neuroinflammation levels in the NAc, or in other embodiments in the VTA, or in other embodiments in both regions, in rats subjected to cocaine addiction and cocaine-primed reinstatement as described in Example 11. In certain embodiments, the neuroinflammation levels are reduced such that they are not significantly different from levels observed in control rats not subjected to cocaine-primed reinstatement. In various embodiments, the aforementioned inflammation levels are obtained 1 week, 2 weeks, 3 weeks, 4 weeks, 6 weeks, 8 weeks, 12 weeks, 18 weeks, 24 weeks, 48 weeks, or 72 weeks after treatment with the ASC.

Further, cocaine increases extracellular levels of glutamate in various brain regions, including the NAc (Berglind W J et al), the VTA (Licata S C et al), and the hippocampus (Pomierny-Chamiolo L et al). According to some embodiments, treatment with the described ASC results in glutamate levels in the NAc, or in other embodiments in the VTA, or in other embodiments in both regions, or in other embodiments in the dorsal and/or ventral hippocampus, prefrontal cortex, striatum, dentate gyrus, or central amigdala, that are 50%, within 40%, within 30%, within 20%, or within 10% of normal levels, in rats subjected to cocaine addiction and cocaine-primed reinstatement as described in Example 11. Each brain region, each level of modulation, and each combination thereof represents a separate embodiment. In various embodiments, the aforementioned glutamate levels are obtained 1 week, 2 weeks, 3 weeks, 4 weeks, 6 weeks, 8 weeks, 12 weeks, 18 weeks, 24 weeks, 48 weeks, or 72 weeks after treatment with the ASC.

ASC and Sources Thereof

In certain embodiments, the described ASC are mesenchymal stromal cells (MSC). These cells may, in some embodiments, be isolated from bone marrow. In further embodiments, the cells are human MSC as defined by The Mesenchymal and Tissue Stem Cell Committee of the International Society for Cellular Therapy (Dominici et al, 2006), based on the following 3 criteria: 1. Plastic-adherence when maintained in standard culture conditions (α minimal essential medium plus 20% fetal bovine serum (FBS)). 2. Expression of the surface molecules CD105, CD73 and CD90, and lack of expression of CD45, CD34, CD14 or CD11b, CD79a or CD19 and HLA-DR. 3. Ability to differentiate into osteoblasts, adipocytes and chondroblasts in vitro.

In certain embodiments, the described MSCs are multipotent stromal cells that can differentiate into a variety of cell types, including: osteoblasts, chondrocytes, myocytes, adipocytes and neuronal-like cells. In vitro differentiation into different types of cells can be achieved by culturing the stem cells with specific growth factors and medium supplements known to induce the desired phenotype. In other embodiments, MSCs are characterized by their potential to induce cell proliferation and tissue development. For example, MSCs possess neurogenic potential through the secretion of growth factors that induce the formation of neurospheres. According to a specific embodiment, the differentiated mesenchymal stem cells (MSCs) are neuronal-differentiated mesenchymal stem cells (nMSCs).

In other embodiments, the described ASC are placenta-derived. Except where indicated otherwise herein, the terms “placenta”, “placental tissue”, and the like refer to any portion of the placenta. Placenta-derived adherent cells may be obtained, in various embodiments, from either fetal or, in other embodiments, maternal regions of the placenta, or in other embodiments, from both regions. More specific embodiments of maternal sources are the decidua basalis and the decidua parietalis. More specific embodiments of fetal sources are the amnion, the chorion, and the villi. In certain embodiments, tissue specimens are washed in a physiological buffer [e.g., phosphate-buffered saline (PBS) or Hank's buffer]. In certain embodiments, the placental tissue from which cells are harvested includes at least one of the chorionic and decidua regions of the placenta, or, in still other embodiments, both the chorionic and decidua regions of the placenta. More specific embodiments of chorionic regions are chorionic mesenchymal and chorionic trophoblastic tissue. More specific embodiments of decidua are decidua basalis, decidua capsularis, and decidua parietalis.

Single-cell suspensions can be made, in other embodiments, by treating the tissue with a digestive enzyme (see below) or/and physical disruption, a non-limiting example of which is mincing and flushing the tissue parts through a nylon filter or by gentle pipetting (e.g. Falcon, Becton, Dickinson, San Jose, Calif.) with washing medium. In some embodiments, the tissue treatment includes use of a DNAse, a non-limiting example of which is Benzonase from Merck.

Placental cells may be obtained, in various embodiments, from a full-term or pre-term placenta. In some embodiments, the placental tissue is optionally minced, followed by enzymatic digestion. Optionally, residual blood is removed from the placenta before cell harvest. This may be done by a variety of methods known to those skilled in the art, for example by perfusion. The term “perfuse” or “perfusion” as used herein refers to the act of pouring or passaging a fluid over or through an organ or tissue. In certain embodiments, the placental tissue may be from any mammal, while in other embodiments, the placental tissue is human. A convenient source of placental tissue is a post-partum placenta (e.g., less than 10 hours after birth), however, a variety of sources of placental tissue or cells may be contemplated by the skilled person. In other embodiments, the placenta is used within 8 hours, within 6 hours, within 5 hours, within 4 hours, within 3 hours, within 2 hours, or within 1 hour of birth. In certain embodiments, the placenta is kept chilled prior to harvest of the cells. In other embodiments, prepartum placental tissue is used. Such tissue may be obtained, for example, from a chorionic villus sampling or by other methods known in the art. Once placental cells are obtained, they are, in certain embodiments, allowed to adhere to an adherent material (e.g., configured as a surface) to thereby isolate adherent cells. In some embodiments, the donor is 35 years old or younger, while in other embodiments, the donor may be any woman of childbearing age.

Placenta-derived cells can be propagated, in some embodiments, by using a combination of 2D and 3D culturing conditions. Conditions for propagating adherent cells in 2D and 3D culture are further described hereinbelow and in the Examples section which follows.

Those skilled in the art will appreciate in light of the present disclosure that cells may be, in some embodiments, extracted from a tissue (e.g. placenta, BM, or adipose), for example using physical and/or enzymatic tissue disruption, followed by marker-based cell sorting, and then may be subjected to the culturing methods described herein.

In still other embodiments, the cells are a placental cell population that is a mixture of fetal-derived placental ASC (also referred to herein as “fetal ASC” or “fetal cells”) and maternal-derived placental ASC (also referred to herein as “maternal ASC” or “maternal cells”) and contains predominantly maternal cells. In more specific embodiments, the mixture contains at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, at least 99.92%, at least 99.95%, at least 99.96%, at least 99.97%, at least 99.98%, or at least 99.99% maternal cells, or contains between 90-99%, 91-99%, 92-99%, 93-99%, 94-99%, 95-99%, 96-99%, 97-99%, 98-99%, 90-99.5%, 91-99.5%, 92-99.5%, 93-99.5%, 94-99.5%, 95-99.5%, 96-99.5%, 97-99.5%, 98-99.5%, 90-99.9%, 91-99.9%, 92-99.9%, 93-99.9%, 94-99.9%, 95-99.9%, 96-99.9%, 97-99.9%, 98-99.9%, 99-99.9%, 99.2-99.9%, 99.5-99.9%, 99.6-99.9%, 99.7-99.9%, or 99.8-99.9% maternal cells.

Predominantly or completely maternal cell preparations may be obtained by methods known to those skilled in the art, including the protocol detailed in Example 1 and the protocols detailed in PCT Publ. Nos. WO 2007/108003, WO 2009/037690, WO 2009/144720, WO 2010/026575, WO 2011/064669, and WO 2011/132087. The contents of each of these publications are incorporated herein by reference. Predominantly or completely fetal cell preparations may be obtained by methods known to those skilled in the art, including selecting fetal cells via their markers (e.g. a Y chromosome in the case of a male fetus).

In other embodiments, the cells are a placental cell population that does not contain a detectable amount of maternal cells and is thus entirely fetal cells. A detectable amount refers to an amount of cells detectable by FACS, using markers or combinations of markers present on maternal cells but not fetal cells, as described herein. In certain embodiments, “a detectable amount” may refer to at least 0.1%, at least 0.2%, at least 0.3%, at least 0.44%, at least 0.5%, at least 0.6%, at least 0.7%, at least 0.8%, at least 0.9%, or at least 1%.

In still other embodiments, the preparation is a placental cell population that is a mixture of fetal and maternal cells and is enriched for fetal cells. In more specific embodiments, the mixture contains at least 70% fetal cells. In more specific embodiments, at least about 30%, 35%, 40%, 45%, 50%, 55%, 60%, 65%, 75%, 80%, 85%, 90%, 95%, 96%, 97%, 98%, 99% or 100% of the cells are fetal cells. Expression of CD200, as measured by flow cytometry, using an isotype control to define negative expression, can be used as a marker of fetal cells under some conditions. In yet other embodiments, at least 60%, at least 65%, at least 70%, at least 75%, at least 80%, at least 85%, at least 90%, at least 92%, at least 95%, at least 96%, at least 97%, at least 98%, at least 99%, at least 99.5%, at least 99.7%, or at least 99.9% of the described cells are fetal cells.

In more specific embodiments, the mixture contains 20-80% fetal cells; 30-80% fetal cells; 40-80% fetal cells; 50-80% fetal cells; 60-800% fetal cells; 20-90% fetal cells; 30-90% fetal cells; 40-90% fetal cells; 50-90% fetal cells; 60-90% fetal cells; 20-80% maternal cells; 30-80% maternal cells; 40-80% maternal cells; 50-80% maternal cells; 60-80% maternal cells; 20-90% maternal cells; 30-90% maternal cells; 40-90% maternal cells; 50-90% maternal cells; or 60-90% maternal cells.

As used herein, the phrase “adipose tissue” refers to a connective tissue which comprises fat cells (adipocytes). Those skilled in the art will appreciate that adipose tissue-derived ASC may be extracted, in various embodiments, by a variety of methods known to those skilled in the art, for example those described in U.S. Pat. No. 6,153,432, which is incorporated herein by reference. The adipose tissue may be derived, in other embodiments, from omental/visceral, mammary, gonadal, or other adipose tissue sites. In some embodiments, the adipose can be isolated by liposuction. In other embodiments, ASC may be derived from adipose tissue by treating the tissue with a digestive enzyme (non-limiting examples of which are collagenase, trypsin, dispase, hyaluronidase or DNAse): and ethylenediaminetetra-acetic acid (EDTA). The cells may be, in some embodiments, subjected to physical disruption, for example using a nylon or cheesecloth mesh filter. In other embodiments, the cells are subjected to differential centrifugation directly in media or over a Ficoll or Percoll or other particulate gradient (see U.S. Pat. No. 7,078,230, which is incorporated herein by reference). As provided herein, neuronal differentiation of adipose cells can be achieved. Those skilled in the art will appreciate that neuronal differentiation can be assayed e.g. by treatment for 3 weeks with a solution containing DMEM/Opti-MEM supplemented with F12 and B27 (5%), basic fibroblast growth factor (FGFb, 50 ng/ml), sonic hedgehog (SHH, 250 ng/ml) and epidermal growth factor (EGF, 50 ng/ml).

In still other embodiments, the ASC are derived from BM. Those skilled in the art will appreciate that BM-ASC can be obtained by Ficoll® extraction to remove red blood cells. During this process, fresh BM is diluted 5:14 in isolation buffer (PBS+2% FBS+2 mM EDTA) and spun down at 300×g for 30 minutes. The interface layer containing the mononuclear cells is then removed and resuspended in 40 ml cold isolation buffer, which is then centrifuged again at 300×g for 10 minutes. The resulting cells are then optionally resuspended in expansion medium and plated on a tissue culture apparatus. As provided herein, factors secreted by BM-MSC are able to support neurosphere development in a rat neonatal cortical cell culture. Those skilled in the art will appreciate that support of neurosphere development can be assayed e.g. by production of MSC-CM (e.g. in serum-free high glucose DMEM) for 24 hours, and use of the filtered CM supplemented with 1% B27 supplement (Invitrogen, Carlsbad, Calif., USA) to stimulate hippocampal neurogenesis

In yet other embodiments, the ASC are derived from peripheral blood; umbilical cord blood; synovial fluid; synovial membranes; spleen; thymus; mucosa (for example nasal mucosa); limbal stroma; ligaments, for example the periodontal ligament; scalp; hair follicles, testicles; embryonic yolk sac; and amniotic fluid. In some embodiments, the ASC are human ASC, while in other embodiments, they may be animal ASC.

In a further aspect of the present invention, the placental ASC, or in other embodiments MSC, are loaded with gold nanoparticles (GNPs), which allow, in some embodiments, non-invasive, real-time tracking of the cells in the brain. According to one embodiment, the GNPs carry at least one therapeutic compound. In more specific embodiments, the compound is locally released in a controlled release manner, e.g. in specific regions of the brain, e.g. any of the regions mentioned herein.

In another embodiment, the described cell population has been produced by expanding a population of ASC in a medium that contains less than 5% animal serum. In certain embodiments, the aforementioned medium contains less than 4% animal serum; less than 3% animal serum; less than 2% animal serum; less than 1% animal serum; less than 0.5% animal serum; less than 0.3% animal serum; less than 0.2% animal serum; or less than 0.1% animal serum. In other embodiments, the medium does not contain animal serum. In other embodiments, the medium is a defined medium to which no serum has been added. Low-serum and serum-free media are collectively referred to as “serum-deficient medium/media”.

Those skilled in the art will appreciate that reference herein to animal serum includes serum from a variety of species, provided that the serum stimulates expansion of the ASC population. In certain embodiments, the serum is mammalian serum, non-limiting examples of which are human serum, bovine serum (e.g. fetal bovine serum and calf bovine serum), equine serum, goat serum, and porcine serum.

In certain embodiments, the serum-deficient medium is supplemented with factors intended to stimulate cell expansion in the absence of serum. Such medium is referred to herein as serum-replacement medium or SRM, and its use, for example in cell culture and expansion, is known in the art, and is described, for example, in Kinzebach et al.

In other embodiments, the serum-deficient medium contains one or more growth factors. In certain embodiments, the growth factors, individually or, in other embodiments collectively, induce cell expansion in culture. In other embodiments, the growth factors, individually or, in other embodiments collectively, induce cell expansion in culture without differentiation.

In more specific embodiments, the factor(s) contained in the SRM is selected from a FGF, TGF-beta (Uniprot accession no. P01137), transferrin (e.g. serotransferrin or lactotransferrin; Uniprot accession nos. P02787 and P02788), insulin (Uniprot accession no. P01308), EGF (epidermal growth factor; Uniprot accession no. P01133), and/or PDGF (platelet-derived growth factor, including any combination of subunits A and B; Uniprot accession nos. P04085 and P01127), each of which represents a separate embodiment. A non-limiting example of PDGF is PDGF-BB.

Except where indicated otherwise, reference herein to a protein includes all its isoforms functional fragments thereof, and mimetics thereof. Such reference also includes homologues from a variety of species, provided that the protein acts on the target cells in a similar fashion to the homologue from the same species as the target cells. For example, if human cells are being expanded, reference to bFGF would also include any non-human bFGF that stimulates proliferation of human cells. Those skilled in the art will appreciate that, even in the case of human cells, the aforementioned proteins need not be human proteins, since many non-human (e.g. animal) proteins are active on human cells. Similarly, the use of modified proteins that have similar activity to the native forms falls within the scope of the described methods and compositions.

The FGF (fibroblast growth factor) family includes a number of proteins that are described in Imamura. A non-limiting example is bFGF (Uniprot accession no. P09038; accessed on May 22, 2017).

In other embodiments, the SRM comprises an FGF and TGF-beta. In still other embodiments, the medium comprises an FGF, TGF-beta, and PDGF. In more specific embodiments, the medium further comprises transferrin, insulin, or both transferrin and insulin. Alternatively or in addition, the medium further comprises oleic acid.

In still other embodiments, the SRM comprises an FGF and EGF. In still other embodiments, the medium further comprises transferrin, insulin, or both transferrin and insulin.

SRM formulations include MSC Nutristem® XF (Biological Industries); Stempro® SFM and Stempro® SFM-XF (Thermo Fisher Scientific); PPRF-msc6; D-hESF10; TheraPEAK™ MSCGM-CD™ (Lonza, cat. no. 190632); and MesenCult-XF (Stem Cell Technologies, cat. no. 5429). The StemPro® media contain PDGF-BB, bFGF, and TGF-β, and insulin (Chase et al). The composition of PPRF-msc6 is described in US 2010/0015710, which is incorporated herein by reference. D-hESF10 contains insulin (10 micrograms per milliliter [also referred to as mcg/ml]); transferrin (5 mcg/ml); oleic acid conjugated with bovine albumin (9.4 mcg/ml); FGF-2 (also referred to as bFGF basic fibroblast growth factor) (10 ng/ml); and TGF-β1 (5 ng/ml), as well as heparin (1 mg/ml) and standard medium components (Mimura et al).

As provided herein, ASC were expanded in Stempro® SFM-XF. MSC Nutristem® XF was also tested and yielded similar results. Additionally, an in-house medium was produced and tested, containing DMEM/F-12 supplemented with 50 ng/ml PDGF-BB, 15 ng/ml bFGF, and 2 ng/ml TGF-β. This medium yielded similar results to Stempro® SFM-XF. DMEM/F-12 is a known basal medium, available commercially from Thermo Fisher Scientific (cat. no. 10565018).

Another SRM formulation is described in Rajaraman G et al and contains FGF-2 (10 ng/ml); epidermal growth factor (EGF) (10 ng/ml); 0.5% BSA; Insulin (10 mcg/ml); transferrin (5.5 mcg/ml); 6.7 ng/mL sodium selenite, sodium pyruvate (11 mcg/ml); heparin (0.1 mg/ml); 10 nM linolenic acid.

In certain embodiments, the described SRM comprises FGF-2, TGF-β (TGF-β, including all isotypes, for example TGFβ1, TGFβ2, and TGFβ3), or a combination thereof. In other embodiments, the SRM comprises bFGF, TGF-β, and PDGF. In still other embodiments, the SRM comprises bFGF and TGF-β, and lacks PDGF-BB. Alternatively or in addition, insulin is also present. In still other embodiments, an additional component selected from ascorbic acid, hydrocortisone and fetuin is present; 2 components selected from ascorbic acid, hydrocortisone and fetuin are present; or ascorbic acid, hydrocortisone and fetuin are all present.

In other embodiments, the described SRM comprises bFGF, TGF-β, and insulin. In additional embodiments, a component selected from transferrin (5 mcg/ml) and oleic acid are present, or both transferrin and oleic acid are present. Oleic acid can be, in some embodiments, conjugated with a protein, a non-limiting example of which is albumin. In some embodiments, the SRM comprises 5-20 ng/ml bFGF, 2-10 ng/ml TGF-β, and 5-20 ng/ml insulin, or, in other embodiments, 7-15 ng/ml bFGF, 3-8 ng/ml TGF-β, and 7-15 ng/ml insulin. In more specific embodiments, a component selected from 2-10 mcg/ml transferrin and 5-20 mcg/ml oleic acid, or in other embodiments, a component selected from 3-8 mcg/ml transferrin and 6-15 mcg/ml oleic acid, or in other embodiments the aforementioned amounts of both components (transferrin and oleic acid) is/are also present.

In yet other embodiments, the described SRM comprises bFGF and EGF. In more specific embodiments, the bFGF and EGF are present at concentrations independently selected from 5-40, 5-30, 5-25, 6-40, 6-30, 6-25, 7-40, 7-30, 7-25, 7-20, 8-, 8-17, 8-15, 8-13, 9-20, 9-17, 9-15, 10-15, 5-20, 5-10, 7-13, 8-12, 9-11, or 10 ng/ml. In certain embodiments, insulin; and/or transferrin is also present. In more specific embodiments, the insulin and transferrin are present at respective concentrations of 5-20 and 2-10; 6-18 and 3-8; or 8-15 and 4-7 mcg/ml. Alternatively or in addition, the SRM further comprises an additional component selected from BSA, selenite (e.g. sodium selenite), pyruvate (e.g. sodium pyruvate); heparin, and linolenic acid. In other embodiments 2 or more, or in other embodiments 3 or more, in other embodiments 4 or more, or in other embodiments all 5 of BSA, selenite, pyruvate, heparin, and linolenic acid are present. In more specific embodiments, the BSA, selenite, pyruvate, heparin, and linolenic acid are present at respective concentrations of 0.1-5%, 2-30 ng/mL, 5-25 mcg/ml, 0.05-0.2 mg/ml, and 5-20 nM; or in other embodiments at respective concentrations of 0.2-2%, 4-10 ng/mL, 7-17 mcg/ml, 0.07-0.15 mg/ml, and 7-15 nM; or in other embodiments the aforementioned amounts or 2 or more, or in other embodiments 3 or more, in other embodiments 4 or more, or in other embodiments all 5 of BSA, selenite, pyruvate, heparin, and linolenic acid are present.

In other embodiments, bFGF, where present, is present at a concentration of 1-40, 1-30, 1-20, 2-40, 2-30, 2-20, 3-40, 3-30, 3-20, 3-15, 4-30, 4-20, 4-15, 5-30, 5-20, 5-15, 6-14, 7-14, 8-13, 8-12, 9-11, 9-12, about 10, or 10 nanograms per milliliter (ng/ml).

In other embodiments, EGF, where present, is present at a concentration of 1-40, 1-30, 1-20, 2-40, 2-30, 2-20, 3-40, 3-30, 3-20, 3-15, 4-30, 4-20, 4-15, 5-30, 5-20, 5-15, 6-14, 7-14, 7-25, 7-22, 8-25, 8-22, 9-21, 10-20, 8-13, 8-12, 9-11, 9-12, about 10, or 10 ng/ml.

In other embodiments, TGF-β, where present, is present at a concentration of 1-25, 2-25, 3-25, 4-25, 5-25, 1-20, 1-15, 1-10, 1-8, 1-7, 1-6, 1-5, 2-20, 2-15, 2-10, 3-20, 3-15, 3-10, 3-8, 3-7, 4-8, 4-7, 4-6, 4.5-5.5, about 5, or 5 ng/ml.

In other embodiments, PDGF, where present, is present at a concentration of 1-50, 1-40, 1-30, 1-20, 1-15, 1-10, 1-8, 1-7, 1-6, 1-5, 1-4, 1-3, 1-2, 2-50, 2-40, 2-30, 2-20, 2-15, 2-10, 2-8, 2-7, 2-6, 2-5, 2-4, 3-50, 3-40, 3-30, 3-20, 3-15, 3-10, 3-8, 3-7, 3-6, 3-5, 3-4, 4-40, 4-30, 4-20, 5-40, 5-30, 5-20, 5-15, 5-12, 5-10, 10-20, 10-18, 10-16, or 10-15, 2-20, about 2, about 3, about 5, about 10, about 15, about 20, 2, 3, 5, 10, 15, or 20 ng/mL.

In still other embodiments, the ASC are expanded in a multi-step process, including the steps of (a) incubating a population of ASC in a serum-deficient medium, thereby obtaining a first expanded cell population; and (b) incubating the first expanded cell population in a second medium, wherein the second medium contains at least 10% animal serum.

The aforementioned second medium, in some embodiments, contains an animal serum content of 5-25%, 6-25%, 7-25%, 8-25%, 9-25%, 10-25%, 11-25%, 12-25%, 13-25%, 14-25%, 15-25%, 10-24%, 10-23%, 10-22%, 10-21%, 10-20%, 11-19%, 12-18%, 13-17%, 16-24%, 17-23%, or 18-22%. In other embodiments, the second medium contains at least 11%, 12%, 13%, 14%, 15%, 16%, 17%, 18%, 19%, or 20% animal serum. In certain embodiments, the second medium does not contain added growth factors, other than those present in the animal serum added thereto.

In still other embodiments, the described methods are preceded by an earlier step wherein cells are cultured in serum-containing medium, prior to culturing in a serum-deficient medium. The serum-containing medium can be, in certain embodiments, any standard growth medium. Non-limiting examples, for exemplary purposes only, are DMEM+10% FBS and DMEM+5% human serum. A non-limiting example of these embodiments is use of standard growth medium to incubate and expand cells, for a limited number of passages (e.g. 1-3 passages, or in other embodiments 2-10 doublings) following their extraction from the source tissue, followed by expansion in serum-deficient medium, which is, in some embodiments, in turn followed by further expansion in serum-containing medium. As provided herein, the initial use of serum-containing medium, for example after extraction, facilitates, in some scenarios, initial attachment and expansion of cells after their extraction. In certain embodiments, the earlier step is performed on a 2D substrate.

In some embodiments, the step of incubating an ASC population in serum-deficient medium is performed on a 2D substrate; and at least a portion of the subsequent step (incubating the expanded cell population in a serum-containing medium) is performed on a 3D substrate. In certain embodiments, the 3D substrate is in a bioreactor. Alternatively or in addition, the 3D substrate is a synthetic adherent material. These embodiments of methods may be freely combined with any of the described embodiments of bioreactors, adherent materials, and/or 3D carriers and substrates. In still other embodiments, the aforementioned subsequent step is initiated on a 2D substrate for a duration of at least 2, at least 3, at least 4, at least 5, at least 6, 2-10, 3-10, 4-10, 5-10, 2-8, 3-8, 4-8, or 5-8 cell doublings, before performing additional expansion in a serum-containing medium on a 3D substrate. The 2D substrate on which the subsequent step is initiated may be the same or different from the 2D substrate on which the described earlier step was performed, where applicable.

Use of Induced ASC

In certain embodiments, the described ASC have been treated to make them suitable for treating a neurological disease, for example a neurodegenerative disease. Such treatment is referred to herein as “induction”, and the cells so treated as “induced” cells. As described herein, induction can comprise, in various embodiments, expansion on a 3D substrate, incubation in medium comprising induction agents, or a combination thereof. In certain embodiments, the induction results in secretion of increased amounts of neurotrophic and/or neuroprotective growth factors.

Embodiments of induced ASC are described in PCT/IB2018/052806 (to Niva Shraga Heled and Rachel Ofir), which is incorporated herein in its entirety.

As provided herein, ASC can be induced by incubation with medium comprising agents that cause them to secrete neurotrophic and/or neuroprotective factors. Such additives may be referred to herein as induction agents, or simply “agents”. Examples of secreted factors are BDNF (brain derived neurotrophic factor; Uniprot Accession No. P23560), GDNF (glial cell line derived neurotrophic factor; Uniprot Accession No. P39905), bFGF (basic fibroblast growth factor; Uniprot accession no. P09038), NGF (nerve growth factor; Uniprot Accession No. P01138), VEGF (vascular endothelial growth factor; Uniprot Accession No. P156), HGF (hepatocyte growth factor; Uniprot Accession No. P08581), and LIF (Leukemia inhibitory factor; Uniprot Accession No. P15018). In certain embodiments, the secreted factors comprise one or more of BDNF, GDNF, bFGF, NGF, VEGF, and HGF. Non-limiting examples of induction agents are cocktails described herein, for example cocktails containing heparin and cAMP. Uniprot Accession Numbers in this paragraph were accessed on May 22, 2017.

It is also provided herein that ASC can be activated by expansion on a 3D substrate, a non-limiting example of which is a carrier comprising a fibrous matrix. The expansion medium may be any of the media described herein, each of which represents a separate embodiment of the present invention.

Induction Agents

As a non-limiting example, ASC can be induced by incubation in a medium comprising heparin and cAMP or an analogue thereof. In certain embodiments, a cAMP analogue described herein is a cell-permeable cAMP analog, non-limiting examples of which are dibutyryl cyclic AMP (dbcAMP), 6-Bnz-cAMP (e.g. provided as a sodium salt) (Tocris Bioscience [Bristol, UK)], cat. no. 5255), cAMPS-Sp, (e.g. provided as a triethylammonium salt) (Tocris, cat. no. 1333), and 8-Bromo-cAMP, (e.g. provided as a sodium salt) (Tocris, cat. no. 1140). In some embodiments, the medium is serum-free. In other embodiments, the medium contains serum, which may be, in more specific embodiments, at any of the concentrations mentioned herein.

In more specific embodiments, the concentration of heparin in the medium is 10-200, 10-180, 10-160, 10-140, 10-120, 10-110, 10-100, 15-200, 15-180, 15-160, 15-140, 15-120, 15-110, 15-100, 20-200, 20-180, 20-160, 20-140, 20-120, 20-110, 20-100, 30-200, 30-180, 30-160, 30-140, 30-120, 30-110, 30-100, 40-200, 40-180, 40-160, 40-140, 40-120, 40-110, 40-100, 50-200, 50-180, 50-160, 50-140, 50-120, 50-110, 50-100, 10-50, 15-50, 20-50, 25-50, 30-50, 40-50, 20-80, 25-75, 30-70, 35-75, 40-60, 45-55, 47-53, 48-52, 49-51, about 50, or 50 mcg/ml (micrograms per ml).

In other embodiments, the concentration of the described cAMP or analogue thereof in the medium is 500-2500 mcM (micromolar). In various other embodiments, it is 200-5000, 200-4000, 200-3000, 200-2500, 200-2000, 200-1500, 200-1200, 200-1000, 300-5000, 300-4000, 300-3000, 300-2500, 300-2000, 300-1500, 300-1200, 300-1000, 400-5000, 400-4000, 400-3000, 400-2500, 400-2000, 400-1500, 400-1200, 400-1000, 500-5000, 500-4000, 500-3000, 500-2000, 500-1500, 500-1200, 500-1000, 600-5000, 600-4000, 600-3000, 600-2500, 600-2000, 600-1500, 600-1200, 600-1000, 800-5000, 800-4000, 800-3000, 800-2500, 800-2000, 800-1500, 800-1200, 800-1000, 1000-5000, 1000-4000, 1000-3000, 1000-2500, 1000-2000, 1000-1500, 1000-1200, 250-1000, 300-1000, 350-1000, 400-1000, 500-1000, 600-1000, 700-1000, 800-1000, 900-1000, 500-1500, 500-1800, 600-1400, 600-1800, 700-1300, 700-1400, 700-1500, 800-1200, 900-1100, 950-1050, about 1000, or 1000 mcM.

In still other embodiments, the medium comprises cAMP, or an analogue thereof, for example dbcAMP, at a concentration of 500-2500 mcM and heparin at a concentration of 10-200 mcg/ml. In other embodiments, the respective concentrations of cAMP or an analogue thereof and heparin are 600-2000 mcM and 20-150 mcg/ml; 700-1800 mcM and 25-140 mcg/ml; 800-1600 mcM and 30-120 mcg/ml; 800-1400 mcM and 35-100 mcg/ml; 800-1200 mcM and 35-80 mcg/ml; or 800-1200 mcM and 35-70 mcg/ml.

In certain embodiments, the medium further comprises (in addition to heparin and cAMP or an analogue thereof) one or more induction agents selected from basic fibroblast growth factor (b-FGF; Uniprot Accession No. P09038); PDGF (platelet-derived growth factor; Uniprot Accession Nos. P04085 [subunit A; exemplified herein] and P01127 [subunit B]); and Neuregulin (e.g. Neuregulin 1, non-limiting examples of which are the isoforms HRG-alpha, HRG-beta, HRG-beta2, and HRG-gamma, and the sequences set forth in Uniprot Accession Nos. B7Z 168 (or Q7RTV8), Q7RTW4, Q7RTW3, AOA024QY88, Q7RTW5, and B9EK51). (The Uniprot entries in this paragraph were accessed on Nov. 10, 2015). Alternatively or in addition, the medium further comprises a component selected from (a) progesterone; and (b) a polyamine. In other embodiments, the medium comprises both of aforementioned components (a) and (b). In further embodiments, the medium further comprises an additional component selected from (c) transferrin, non-limiting examples of which are apo-transferrin and holo-transferrin; (d) insulin, non-limiting examples of which are full chain insulin and truncated insulin; and (e) selenite. Non-limiting examples of polyamines are putrescine, spermidine, and spermine. In other embodiments, the medium further comprises (in addition to 1 or, in another embodiment, both of components (a)-(b)), 2 or more of aforementioned components (c)-(e). In still other embodiments, the medium further comprises all 3 or more of components (c)-(e). In yet other embodiments, the medium further comprises 2 or more of aforementioned components (a)-(e). In still other embodiments, the medium further comprises 3 or more of components (a)-(e). In yet other embodiments, the medium further comprises 4 or more of components (a)-(e). In further embodiments, the medium further comprises all 5 of components (a)-(e).

Those skilled in the art will appreciate that the precise sequences of b-FGF, PDGF, Neuregulin, and the other induction agents mentioned herein are not typically critical for carrying out the described methods. Alternative isoforms, functional fragments thereof, mimetics thereof, and proteins from non-human species are often suitable, provided that they exhibit biological effects analogous to the active versions.

In more specific embodiments, the concentration of b-FGF in the medium is 5-100 ng/ml (nanograms per milliliter). In various other embodiments, it is 2-100, 3-100, 7-100, 10-100, 15-100, 20-100, 2-80, 3-80, 5-80, 7-80, 10-80, 15-80, 20-80, 2-50, 3-50, 5-50, 7-50, 10-50, 15-50, 20-50, 2-35, 3-35, 5-35, 7-35, 10-35, 15-35, 20-35, 2-20, 3-20, 5-20, 7-20, 10-20, 15-20, 20-80, 20-50, 20-40, 20-30, 10-30, 10-25, 15-30, 15-35, 15-25, 16-24, 17-23, 18-22, 19-21, about 20, or 20 ng/ml.

In still other embodiments, the medium comprises cAMP, or an analogue thereof, for example dbcAMP, at a concentration of 500-2500 mcM; heparin at a concentration of 10-200 mcg/ml; and b-FGF at a concentration of 5-100 ng/ml. In other embodiments, the respective concentrations of cAMP or an analogue thereof, heparin, and b-FGF are 600-2000 mcM, 20-150 mcg/ml, and 6-80 ng/ml; 700-1800 mcM, 25-140 mcg/ml, and 8-60 ng/ml; 800-1600 mcM, 30-120 mcg/ml, and 10-40 ng/ml; 800-1400 mcM, 35-100 mcg/ml, and 12-35 ng/ml; 800-1200 mcM, 35-80 mcg/ml, and 14-30 ng/ml; or 800-1200 mcM, 35-70 mcg/ml, and 16-25 ng/ml.

In more specific embodiments, the concentration of PDGF is 1-20, 1-18, 1-16, 1-14, 1-12, 1-11, 1-10, 1.5-20, 1.5-18, 1.5-16, 1.5-14, 1.5-12, 1.5-11, 1.5-10, 2-20, 2-18, 2-16, 2-14, 2-12, 2-11, 2-10, 3-20, 3-18, 3-16, 3-14, 3-12, 3-11, 3-10, 4-20, 4-18, 4-16, 4-14, 4-12, 4-11, 4-10, 5-20, 5-18, 5-16, 5-14, 5-12, 5-11, 5-10, 1-5, 1.5-5, 2-5, 2.5-5, 3-5, 4-5, 2-8, 2.5-7.5, 3-7, 3.5-7.5, 4-6, 4.5-5.5, 4.7-5.3, 4.8-5.2, 4.9-5.1, about 5, or 5 ng/ml.

In more specific embodiments, the concentration of Neuregulin is 10-200, 10-180, 10-160, 10-140, 10-120, 10-110, 10-100, 15-200, 15-180, 15-160, 15-140, 15-120, 15-110, 15-100, 20-200, 20-180, 20-160, 20-140, 20-120, 20-110, 20-100, 30-200, 30-180, 30-160, 30-140, 30-120, 30-110, 30-100, 40-200, 40-180, 40-160, 40-140, 40-120, 40-110, 40-100, 50-200, 50-180, 50-160, 50-140, 50-120, 50-110, 50-100, 10-50, 15-50, 20-50, 25-50, 30-50, 40-50, 20-80, 25-75, 30-70, 35-75, 40-60, 45-55, 47-53, 48-52, 49-51, about 50, or 50 ng/ml.

In still other embodiments, the medium further comprises (in addition to heparin and cAMP or an analogue thereof) both (i) an induction agent selected from b-FGF, PDGF, and Neuregulin (each of which represents a separate embodiment); and (ii) an additional component selected from: (a) progesterone; and (b) a polyamine. In other embodiments, the medium comprises both of aforementioned components (a) and (b). In yet other embodiments, the medium further comprises an additional component selected from (c) transferrin, non-limiting examples of which are apo-transferrin and holo transferrin; (d) insulin, non-limiting examples of which are full chain insulin and truncated insulin; and (e) selenite. Non-limiting examples of polyamines are putrescine, spermidine, and spermine. In other embodiments, the medium further comprises—in addition to a component selected from b-FGF, PDGF, and Neuregulin; and 1 or, in another embodiment, both of components (a)-(b)-2 or more of aforementioned components (c)-(e). In still other embodiments, the medium further comprises all 3 or more of components (c)-(e). Non-limiting examples of PDGF are PDGF-AA (exemplified herein), PDGF-BB, and PDGF-AB. In some embodiments, the medium is serum-free. In other embodiments, the medium contains serum, which may be, in more specific embodiments, at any of the concentrations mentioned herein.

In yet other embodiments, the medium further comprises both (i) b-FGF, PDGF, or Neuregulin; and (ii) 2 or more of aforementioned components (a)-(e). In yet other embodiments, the medium further comprises both b-FGF, PDGF, or Neuregulin; and 3 or more of components (a)-(e). In yet other embodiments, the medium further comprises both b-FGF, PDGF, or Neuregulin; and 4 or more of components (a)-(e). In yet other embodiments, the medium further comprises both b-FGF, PDGF, or Neuregulin; and all 5 of components (a)-(e).

In certain embodiments, the concentration of additional component (a) is 2-50, 3-50, 4-50, 5-50, 8-50, 10-50, 2-40, 3-40, 4-40, 5-40, 8-40, 10-40, 2-30, 3-30, 4-30, 5-30, 8-30, 10-30, 2-20, 3-20, 4-20, 5-20, 8-20, 10-20, 2-10, 3-10, 4-10, 5-10, 7-10, 8-10, 9-10, 5-15, 6-14, 7-13, 8-12, 9-11, 7-15, 8-20, about 10, or 10 nM (nanomolar).

Alternatively or in addition, the concentration of additional component (b) is 1-20, 1-18, 1-16, 1-14, 1-12, 1-11, 1-10, 1.5-20, 1.5-18, 1.5-16, 1.5-14, 1.5-12, 1.5-11, 1.5-10, 2-20, 2-18, 2-16, 2-14, 2-12, 2-11, 2-10, 3-20, 3-18, 3-16, 3-14, 3-12, 3-11, 3-10, 4-20, 4-18, 4-16, 4-14, 4-12, 4-11, 4-10, 5-20, 5-18, 5-16, 5-14, 5-12, 5-11, 5-10, 1-5, 1.5-5, 2-5, 2.5-5, 3-5, 4-5, 2-8, 2.5-7.5, 3-7, 3.5-7.5, 4-6, 4.5-5.5, 4.7-5.3, 4.8-5.2, 4.9-5.1, about 5, or 5 mg/L (milligrams per liter).

Alternatively or in addition, the concentration of additional component (c) is 1-20, 1-18, 1-16, 1-14, 1-12, 1-11, 1-10, 1.5-20, 1.5-18, 1.5-16, 1.5-14, 1.5-12, 1.5-11, 1.5-10, 2-20, 2-18, 2-16, 2-14, 2-12, 2-11, 2-10, 3-20, 3-18, 3-16, 3-14, 3-12, 3-11, 3-10, 4-20, 4-18, 4-16, 4-14, 4-12, 4-11, 4-10, 5-20, 5-18, 5-16, 5-14, 5-12, 5-11, 5-10, 6-20, 6-18, 6-16, 6-14, 6-12, 6-11, 6-10, 1-6, 1.5-6, 2-6, 2.5-6, 3-6, 4-6, 2-8, 3-7.5, 3-8, 4-8, 4-7, 5-7, 5.5-6.5, about 6, or 6 ng/L.

Alternatively or in addition, the concentration of additional component (d) is 20-500, 20-400, 20-300, 20-250, 20-200, 20-150, 20-120, 20-100, 30-500, 30-400, 30-300, 30-250, 30-200, 30-150, 30-120, 30-100, 40-500, 40-400, 40-300, 40-250, 40-200, 40-150, 40-120, 40-100, 50-500, 50-400, 50-300, 50-250, 50-200, 50-150, 50-120, 50-100, 60-500, 60-400, 60-300, 60-250, 60-200, 60-150, 60-120, 60-100, 80-500, 80-400, 80-300, 80-250, 80-200, 80-150, 80-120, 80-100, 100-500, 100-400, 100-300, 100-250, 100-200, 100-150, 100-120, 25-100, 30-100, 35-100, 40-100, 50-100, 60-100, 70-100, 80-100, 90-100, 50-150, 50-180, 60-140, 60-180, 70-130, 70-140, 70-150, 80-120, 90-110, 95-105, about 100, or 100 nM.

Alternatively or in addition, the concentration of additional component (e) is 100-2000, 100-1800, 100-1600, 100-1400, 100-1200, 100-1100, 100-1000, 1.500-2000, 1.500-1800, 1.500-1600, 1.500-1400, 1.500-1200, 1.500-1100, 1.500-1000, 200-2000, 200-1800, 200-1600, 200-1400, 200-1200, 200-1100, 200-1000, 300-2000, 300-1800, 300-1600, 300-1400, 300-1200, 300-1100, 300-1000, 400-2000, 400-1800, 400-1600, 400-1400, 400-1200, 400-1100, 400-1000, 500-2000, 500-1800, 500-1600, 500-1400, 500-1200, 500-1100, 500-1000, 100-500, 1.500-500, 200-500, 2.500-500, 300-500, 400-500, 200-800, 2.500-7.500, 300-700, 3.500-7.500, 400-600, 4.500-5.500, 4.700-5.300, 4.800-5.200, 4.900-5.100, about 500, or 500 ng/L.

In still other embodiments, N-2 is present in the medium at between 0.2-5, 0.2-4, 0.2-3, 0.2-2, 0.2-1.5, 0.2-1.2, 0.2-1, 0.3-5, 0.3-4, 0.3-3, 0.3-2, 0.3-1.5, 0.3-1.2, 0.3-1, 0.5-5, 0.5-4, 0.5-3, 0.5-2, 0.5-1.5, 0.5-1.2, 0.5-1, 0.6-1.4, 0.6-1.5, 0.7-1.3, 0.7-1.4, 0.8-1.2, 0.8-1.5, 0.8-1.4, 0.9-1.1, about 1, or 1× concentration, where 1× concentration is the usual recommended concentration. N-2 animal-free cell culture supplement is commercially available from ThermoFisher Scientific, Cat. #1752048. 100× N-2 contains 1 mM human transferrin (holo), 500 mg/L (milligrams per liter) Insulin Recombinant Full Chain, 0.63 mg/L progesterone, 10 mM putrescine, and 0.52 mg/L selenite. 1× N-2 contains 10 micromolar (mcM) transferrin, 5 mg/L Insulin, 6.3 mcg/L progesterone, 100 mcM putrescine, and 5.2 mcg/L selenite. In some embodiments, the medium is serum-free. In other embodiments, the medium contains serum, which may be, in more specific embodiments, at any of the concentrations mentioned herein.

In still other embodiments, the medium comprises cAMP, or an analogue thereof, for example dbcAMP, at a concentration of 500-2500 mcM; heparin at a concentration of 10-200 mcg/ml; b-FGF at a concentration of 5-100 ng/ml; and one or more of (a) progesterone at a concentration of 2-20 mcg/L, and (b) a polyamine (e.g. putrescine) at a concentration of 30-300 mcM. In other embodiments, the respective concentrations of cAMP or an analogue thereof, heparin, b-FGF, progesterone, and polyamine are 600-2000 mcM, 20-150 mcg/ml, 6-80 ng/ml, 3-15 mcg/mL and 50-200 mcM; 700-1800 mcM, 25-140 mcg/ml, 8-60 ng/ml, 4-12 mcg/mL and 60-180 mcM; 800-1600 mcM, 30-120 mcg/ml, 10-40 ng/ml, 4-10 mcg/mL and 70-160 mcM; 800-1400 mcM, 35-100 mcg/ml, 12-35 ng/ml, 4-10 mcg/mL and 70-140 mcM; 800-1200 mcM, 35-80 mcg/ml, 14-30 ng/ml, 4-10 mcg/mL and 70-140 mcM; or 800-1200 mcM, 35-70 mcg/ml, 16-25 ng/ml, 5-8 mcg/mL and 80-120 mcM. In certain embodiments, both progesterone and a polyamine are present. In some embodiments, the medium is serum-free. In other embodiments, the medium contains serum, which may be, in more specific embodiments, at any of the concentrations mentioned herein.

In certain embodiments, the induction of ASC is performed on a 2D substrate. In other embodiments, the induction is performed on a 3D substrate. Unless indicated otherwise, a 3D substrate culture apparatus used for induction may be any apparatus mentioned herein, each of which represents a separate embodiment.

In some embodiments, the ASC are expanded ex vivo prior to the step of inducing. For example, the cells may be incubated in a medium lacking one or more induction agents. In more specific embodiments, the ASC are expanded on a 2D substrate, and then induced on a 3D substrate. In some embodiments, the 2D substrate is used for expansion, and the 3D substrate is subsequently used, exclusively for the induction stage. In other embodiments, the 2D substrate is used for expansion, and the 3D substrate is subsequently used for additional cell expansion, followed by cell induction in the 3D substrate.

In other embodiments, the ASC are induced to secrete a neurotrophic or neuroprotective growth factor by incubation in a bioreactor, optionally while adhered to a 3D growth substrate and/or in the presence of serum. The medium may be any of the media described herein, each of which represents a separate embodiment.

In other embodiments, the ASC are induced to secrete a neurotrophic or neuroprotective growth factor by incubation in a medium comprising heparin and cAMP or an analogue thereof. In other embodiments, the medium comprises basic FGF and cAMP or an analogue thereof. In certain embodiments, a cAMP analogue described herein is a cell-permeable cAMP analog, as described herein. In some embodiments, the medium is serum-free. In other embodiments, the medium contains serum, which may be, in more specific embodiments, at any of the concentrations mentioned herein, each of which represents a separate embodiment. In certain embodiments, incubation with heparin and cAMP is performed on a 2D substrate. In other embodiments, the incubation is performed on a 3D substrate. Unless indicated otherwise, a 3D substrate used for induction may be any culture apparatus mentioned herein, each of which represents a separate embodiment.

In other embodiments, the cells are initially expanded in a medium lacking heparin and cAMP, and the medium is exchanged for a medium comprising heparin and cAMP for an additional period of time. In certain embodiments, the cells are incubated in the heparin-and-cAMP-containing induction cocktail for 12-72 hours, in other embodiments, 18-72 hours, 18-60 hours, 18-48 hours, 18-36 hours, 20-36 hours, 20-30 hours, or 20-28 hours. The heparin-containing induction cocktail may be any cocktail described herein, each of which represents a separate embodiment. Incubation in the heparin-containing cocktail may be, in various embodiments, on a 2D- or 3D-substrate.

In certain embodiments, ASC are induced by the described methods to secrete increased levels of neurotrophic and/or neuroprotective growth factors.

In other embodiments, the step of incubating ASC in a bioreactor is preceded by incubation in serum-free medium (SFM), or, in more specific embodiments, serum replacement medium (SRM; defined herein). In yet other embodiments, the step of incubating ASC with heparin and cAMP is preceded by incubation in SRM, on in other embodiments SFM. In certain embodiments, the incubation in SRM or SFM begins from the stage of extraction from the placenta. In other embodiments, serum-containing medium is initially used, and then culturing in SRM or SFM is commenced within 5 days after extraction, or in other embodiments 1 passage after extraction, or in other embodiments prior to the first passage after extraction. In certain embodiments, the initial serum-containing medium does not comprise added heparin or cAMP. In certain embodiments, the incubation in SRM or SFM continues in a tissue culture apparatus for at least 3 passages, at least 4 passages, at least 5 passages, or at least 6 passages.

As mentioned, in some embodiments, an induction medium (comprising an induction cocktail) is added following the incubation in SRM, and the cells are incubated for an additional period of time. In certain embodiments, the induction cocktail contains heparin and/or cAMP or an analogue thereof. Alternatively or in addition, the cells are incubated in the induction cocktail for 12-72 hours, in other embodiments, 18-72 hours, 18-60 hours, 18-48 hours, 18-36 hours, 20-36 hours, 20-30 hours, or 20-28 hours. The induction cocktail may be any cocktail described herein each of which represents a separate embodiment. Incubation in the cocktail may be, in various embodiments, on a 2D- or 3D-substrate. In certain embodiments, ASC are incubated in a serum-containing medium between the SRM and the induction medium.

In other embodiments, serum-containing medium is used for the initial 2-5 population doublings, on in other embodiments 2-20, 2-15, 2-10, 2-8, or 2-6 population doublings after the first passage. Those skilled in the art will appreciate that it may be difficult to determine an exact population doubling level (PDL) between extraction of cells from tissue and the first passage. In such case, if necessary the population doublings at this first stage may be estimated. Typical population doubling values prior to the first passage are below 5, often ranging from 2-5. In certain embodiments, the initial serum-containing medium does not comprise added heparin or cAMP.

In certain embodiments, the described step of incubating the ASC population in serum-free medium, or in other embodiments in SRM, is performed for at least 12, at least 15, at least 17, at least 18, 12-30, 12-25, 15-30, 15-25, 16-25, 17-25, or 18-25 doublings.

In other embodiments, the ASC population is incubated in SFM, or in other embodiments in SRM, for a defined number of passages, for example 1-4, 1-3, 1-2, 2-4, or 2-3; or a defined number of population doublings, for example at least 4, at least 5, at least 6, at least 7, at least 8, 4-10, 4-9, 4-8, 5-10, 5-9, or 5-8. The cells are then cryopreserved, then subjected to additional culturing in SRM or SFM, prior to induction. In some embodiments, the additional culturing in SRM or SFM is performed for at least 6, at least 7, at least 8, at least 9, at least 10, 6-20, 7-20, 8-20, 9-20, 10-20, 6-15, 7-15, 8-15, 9-15, or 10-15 population doublings. Alternatively, the additional culturing in SRM is performed for 2-3 passages, or in other embodiments at least 1, at least 2, at least 3, 1-5, 1-4, 1-3, 2-5, or 2-4 passages.

Each of the described embodiments of culturing ASC in SFM or in SRM may be followed by incubation in a bioreactor, in in other embodiments, incubation with heparin and cAMP.

In some embodiments, ASC are incubated in SRM (or in other embodiments SFM), followed by serum-containing medium, prior to induction by incubation in a bioreactor, in in other embodiments, by incubation with heparin and cAMP. In other embodiments, serum-containing medium is initially used, then ASC are incubated in SRM (or SFM), then once again in serum-containing medium, prior to induction by incubation in a bioreactor, in in other embodiments, incubation with heparin and cAMP. In other embodiments, the induction is performed in serum-containing medium comprising heparin and cAMP.

In certain embodiments, the ASC are expanded in SRM (or SFM) on a 2D substrate, followed by induction on a 3D substrate. In other embodiments, the 2D substrate is used for expansion, and the 3D substrate is subsequently used for additional cell expansion, followed by induction in the 3D substrate. In other embodiments, SRM (or SFM) is utilized for part of incubation on a 2D matrix, after which serum-containing medium is utilized for the remainder of incubation on a 2D matrix, and also for incubation on a 3D matrix-which occurs, in some embodiments, in a bioreactor-after which the ASC are induced with heparin and cAMP. In other embodiments, the incubation in SRM (or SFM) continues until seeding of the cells in serum-containing medium on a 3D matrix-which occurs, in some embodiments, in a bioreactor-after which the ASC are then induced with heparin and cAMP. In other embodiments, the induction is performed in serum-containing medium comprising heparin and cAMP.

The described serum-containing medium, in certain embodiments, contains 5-30% serum (non-limiting examples of which are fetal bovine serum and fetal calf serum). In more specific embodiments, the medium contains over 10% serum; 10-30% serum; 12-28% serum; 14-26% serum; 16-24% serum; 17-23% serum; 18-22% serum; 19-21% serum; about 20% serum; or 20% serum. The serum-containing medium used at the described stages may be varied independently, and each possibility represents a separate embodiment.

It is clarified that the embodiments wherein the serum-containing medium comprises one or more of the aforementioned induction agents (non-limiting examples of which are bFGF, dbcAMP, heparin, and N-2 supplement) are not excluded from the present disclosure. In still other embodiments, the serum-containing medium further comprises contains N-2 or at least 2 components thereof (for example progesterone and a polyamine (e.g. putrescine). In more specific embodiments, the serum-containing medium further comprises at least 3, at least 4, or all 5 N-2 components.

Alternatively, incubation in SRM (as described) is followed by incubation in a subsequent medium which is serum-free and does not contain any of the following: heparin, cAMP or an analogue thereof, b-FGF, PDGF, or Neuregulin. In certain embodiments, the subsequent medium comprises added cytokines, not including any of heparin, cAMP or an analogue thereof, b-FGF, PDGF, or Neuregulin. Incubation in SRM is, in some embodiments, preceded by incubation in serum-containing medium. In any case, incubation in the described subsequent medium is followed by induction by incubation in a bioreactor, or in other embodiments, by incubation with heparin and cAMP, as described herein.

Surface markers and additional characteristics of ASC Alternatively or additionally, the described placental ASC may express a marker or a collection of markers (e.g. surface marker) characteristic of MSC or mesenchymal-like stromal cells. In some embodiments, the ASC express some or all of the following markers: CD105 (UniProtKB Accession No. P17813), CD29 (UniProtKB Accession No. P05556), CD44 (UniProtKB Accession No. P16070), CD73 (UniProtKB Accession No. P21589), and CD90 (UniProtKB Accession No. P04216). In some embodiments, the ASC do not express some or all of the following markers: CD3 (e.g. UniProtKB Accession Nos. P09693 [gamma chain] P04234 [delta chain], P07766 [epsilon chain], and P20963 [zeta chain]), CD4 (UniProtKB Accession No. P01730), CD11b (UniProtKB Accession No. P11215), CD14 (UniProtKB Accession No. P08571), CD19 (UniProtKB Accession No. P15391), and/or CD34 (UniProtKB Accession No. P28906). In more specific embodiments, the ASC also lack expression of CD5 (UniProtKB Accession No. P06127), CD20 (UniProtKB Accession No. P11836), CD45 (UniProtKB Accession No. P08575), CD79-alpha (UniProtKB Accession No. B5QTD1), CD80 (UniProtKB Accession No. P33681), and/or HLA-DR (e.g. UniProtKB Accession Nos. P04233 [gamma chain], P01903 [alpha chain], and P01911 [beta chain]). The aforementioned, non-limiting marker expression patterns were found in certain maternal placental cell populations that were expanded on 3D substrates. All UniProtKB entries mentioned in this paragraph were accessed on Jul. 7, 2014. Those skilled in the art will appreciate that the presence of complex antigens such as CD3 and HLA-DR may be detected by antibodies recognizing any of their component parts, such as, but not limited to, those described herein.

In some embodiments, the placental ASC possess a marker phenotype that is distinct from bone marrow-mesenchymal stem cells (BM-MSC). Alternatively or in addition, the ASC are positive for expression of CD10 (which occurs, in some embodiments, in both maternal and fetal ASC); are positive for expression of CD49d (which occurs, in some embodiments, at least in maternal ASC); are positive for expression of CD54 (which occurs, in some embodiments, in both maternal and fetal ASC); are bimodal, or in other embodiments positive, for expression of CD56 (which occurs, in some embodiments, in maternal ASC); and/or are negative for expression of CD106. Except where indicated otherwise, bimodal refers to a situation where a significant percentage (e.g. at least 20%) of a population of cells express a marker of interest, and a significant percentage do not express the marker.

In certain embodiments, over 90% of the placental ASC are positive for CD29, CD90, and CD54. In other embodiments, over 85% of the described cells are positive for CD29, CD73, CD90, and CD105. In yet other embodiments, less than 3% of the described cells are positive for CD14, CD19, CD31, CD34, CD39, CD45RA (an isotype of CD45), HLA-DR, Glycophorin A, and CD200; less than 6% of the cells are positive for GlyA; and less than 20% of the cells are positive for SSEA4. In more specific embodiments, over 90% of the described cells are positive for CD29, CD90, and CD54; and over 85% of the cells are positive for CD73 and CD105. In still other embodiments, over 90% of the described cells are positive for CD29, CD90, and CD54; over 85% of the cells are positive for CD73 and CD105; less than 6% of the cells are positive for CD14, CD19, CD31, CD34, CD39, CD45RA, HLA-DR, GlyA, CD200, and GlyA; and less than 20% of the cells are positive for SSEA4. The aforementioned, non-limiting marker expression patterns were found in certain maternal placental cell populations that were expanded on 3D substrates.

In other embodiments, each of CD73, CD29, and CD105 is expressed by more than 90% of the placental ASC; and over 90% (or in other embodiments, over 95%, or in other embodiments, over 98%) of the cells do not differentiate into adipocytes, under conditions where mesenchymal stem cells would differentiate into adipocytes. In some embodiments, as provided herein, the conditions are incubation of adipogenesis induction medium, for example a solution containing 1 mcM dexamethasone, 0.5 mM 3-Isobutyl-1-methylxanthine (IBMX), 10 mcg/ml insulin, and 100 mcM indomethacin, on days 1, 3, 5, 9, 11, 13, 17, 19, and 21; and replacement of the medium with adipogenesis maintenance medium, namely a solution containing 10 mcg/ml insulin, on days 7 and 15, for a total of 25 days (“standard adipogenesis induction conditions”). In yet other embodiments, each of CD34, CD45, CD19, CD14 and HLA-DR is expressed by less than 3% of the cells; and the cells do not differentiate into adipocytes, after incubation under the aforementioned conditions. In other embodiments, each of CD73, CD29, and CD105 is expressed by more than 90% of the cells, each of CD34, CD45, CD19, CD14 and HLA-DR is expressed by less than 3% of the cells; and the cells do not differentiate into adipocytes, after incubation under the aforementioned conditions. In still other embodiments, a modified adipogenesis induction medium, containing 1 mcM dexamethasone, 0.5 mM IBMX, 10 mcg/ml insulin, and 200 mcM indomethacin is used, and the incubation is for a total of 26 days (“standard adipogenesis induction conditions”). The aforementioned solutions will typically contain cell culture medium such as DMEM+10% serum or the like, as will be appreciated by those skilled in the art. The aforementioned, non-limiting phenotypes and marker expression patterns were found in certain maternal placental cell populations that were expanded on 3D substrates.

“Positive” expression of a marker indicates a value higher than the range of the main peak of an isotype control histogram; this term is synonymous herein with characterizing a cell as “express”/“expressing” a marker. “Negative” expression of a marker indicates a value falling within the range of the main peak of an isotype control histogram; this term is synonymous herein with characterizing a cell as “not express”/“not expressing” a marker. “High” expression of a marker, and term “highly express[es]” indicates an expression level that is more than 2 standard deviations higher than the expression peak of an isotype control histogram, or a bell-shaped curve matched to said isotype control histogram.

In still other embodiments, the majority, in other embodiments over 60%, over 70%, over 800%, or over 900% of the expanded cells express CD29, CD73, CD90, and CD105. In yet other embodiments, less than 20%, 15%, or 10% of the described cells express CD3, CD4, CD34, CD39, and CD106. In yet other embodiments, less than 20%, 15%, or 10% of the described cells highly express CD56. In various embodiments, the cell population may be less than 50%, less than 40%, less than 30%, less than 20%, or less than 10%, or less than 5% positive for CD200. In other embodiments, the cell population is more than 50%, more than 60%, more than 70%, more than 80%, more than 90%, more than 95%, more than 97%, more than 98%, more than 99%, or more than 99.5% positive for CD200. In certain embodiments, more than 50% of the cells express, or in other embodiments highly express, CD141 (thrombomodulin; UniProt Accession No. P07204), or in other embodiments SSEA4 (stage-specific embryonic antigen 4, an epitope of ganglioside GL-7 (IV³ NeuAc 2→3 GalGB4); Kannagi R et al), or in other embodiments both markers. Alternatively or in addition, more than 50% of the cells express HLA-A2 (UniProt Accession No. P01892). The aforementioned, non-limiting marker expression patterns were found in certain fetally-derived placental cell populations that were expanded on 3D substrates. The Uniprot Accession Nos. mentioned in the paragraph were accessed on Feb. 8, 2017.

In other embodiments, each of CD29, CD73, CD90, and CD105 is expressed by more than 80% of the cells that have been expanded; and over 90% (or in other embodiments, over 95%, or in other embodiments, over 98%) of the cells do not differentiate into osteocytes, after incubation for 17 days with a solution containing 0.1 mcM dexamethasone, 0.2 mM ascorbic acid, and 10 mM glycerol-2-phosphate, in plates coated with vitronectin and collagen (“standard osteogenesis induction conditions”). In yet other embodiments, each of CD34, CD39, and CD106 is expressed by less than 10% of the cells; less than 20% of the cells highly express CD56; and the cells do not differentiate into osteocytes, after incubation under the aforementioned conditions. In other embodiments, each of CD29, CD73, CD90, and CD105 is expressed by more than 90% of the cells, each of CD34, CD39, and CD106 is expressed by less than 5% of the cells; less than 20%, 15%, or 10% of the cells highly express CD56, and/or the cells do not differentiate into osteocytes, after incubation under the aforementioned conditions. In still other embodiments, the conditions are incubation for 26 days with a solution containing 10 mcM dexamethasone, 0.2 mM ascorbic acid, 10 mM glycerol-2-phosphate, and 10 nM Vitamin D, in plates coated with vitronectin and collagen (“modified osteogenesis induction conditions”). The aforementioned solutions will typically contain cell culture medium such as DMEM+10% serum or the like, as will be appreciated by those skilled in the art. In yet other embodiments, less than 20%, 15%, or 10% of the described cells highly express CD56. In various embodiments, the cell population may be less than 50%, less than 40%, less than 30%, less than 20%, or less than 10%, or less than 5% positive for CD200. In other embodiments, the cell population is more than 50%, more than 60%, more than 70%, more than 80%, more than 90%, more than 95%, more than 97%, more than 98%, more than 99%, or more than 99.5% positive for CD200. In certain embodiments, greater than 50% of the cells highly express CD141, or in other embodiments SSEA4, or in other embodiments both markers. In other embodiments, the cells highly express CD141. Alternatively or in addition, greater than 50% of the cells express HLA-A2. The aforementioned, non-limiting phenotypes and marker expression patterns were found in certain fetally-derived placental cell populations that were expanded on 3D substrates.

In other embodiments, each of CD29, CD73, CD90, and CD105 is expressed by more than 80% of the cells that have been expanded; and over 90% (or in other embodiments, over 95%, or in other embodiments, over 98%) of the cells do not differentiate into adipocytes, after incubation in adipogenesis induction medium, namely a solution containing 1 mcM dexamethasone, 0.5 mM IBMX, 10 mcg/ml insulin, and 100 mcM indomethacin, on days 1, 3, 5, 9, 11, 13, 17, 19, and 21; and replacement of the medium with adipogenesis maintenance medium, namely a solution containing 10 mcg/ml insulin, on days 7 and 15, for a total of 25 days. In yet other embodiments, each of CD34, CD39, and CD106 is expressed by less than 10% of the cells; less than 20% of the cells highly express CD56; and the cells do not differentiate into adipocytes, after incubation under the aforementioned conditions. In other embodiments, each of CD29, CD73, CD90, and CD105 is expressed by more than 90% of the cells, each of CD34, CD39, and CD106 is expressed by less than 5% of the cells; less than 20%, 15%, or 10% of the cells highly express CD56; and the cells do not differentiate into adipocytes, after incubation under the aforementioned conditions. In still other embodiments, a modified adipogenesis induction medium, containing 1 mcM dexamethasone, 0.5 mM IBMX, 10 mcg/ml insulin, and 200 mcM indomethacin is used, and the incubation is for a total of 26 days. In still other embodiments, over 90% of the cells in each population do not differentiate into either adipocytes or osteocytes under the aforementioned standard conditions. In yet other embodiments, over 90% of the cells in each population do not differentiate into either adipocytes or osteocytes under the aforementioned modified conditions. The aforementioned solutions will typically contain cell culture medium such as DMEM+10% serum or the like, as will be appreciated by those skilled in the art. In various embodiments, the cell population may be less than 50%, less than 40%, less than 30%, less than 20%, or less than 10%, or less than 5% positive for CD200. In other embodiments, the cell population is more than 50%, more than 60%, more than 700%, more than 80%, more than 90%, more than 95%, more than 97%, more than 98%, more than 99%, or more than 99.5% positive for CD200. In certain embodiments, greater than 50% of the cells highly express CD141, or in other embodiments SSEA4, or in other embodiments both markers. In other embodiments, the cells highly express CD141. Alternatively or in addition, greater than 50% of the cells express HLA-A2. The aforementioned, non-limiting phenotypes and marker expression patterns were found in certain fetally-derived placental cell populations that were expanded on 3D substrates.

Additionally or alternatively, the placental ASC secrete or express (as appropriate in each case) IL-6 (UniProt identifier P05231), IL-8 (UniProt identifier P10145), eukaryotic translation elongation factor 2 (EEEF2), reticulocalbin 3, EF-hand calcium binding domain (RCN2), and/or calponin 1 basic smooth muscle (CNN1). In more specific embodiments, greater than 50%, in other embodiments greater than 55%, in other embodiments greater than 60%, in other embodiments greater than 65%, in other embodiments greater than 700%, in other embodiments greater than 75%, in other embodiments greater than 80%, in other embodiments greater than 85%, in other embodiments greater than 90%, in other embodiments greater than 95%, in other embodiments greater than 96%, in other embodiments greater than 97%, in other embodiments greater than 98%, in other embodiments greater than 99%, of the cells express or secrete at least one, in other embodiments at least 2, in other embodiments at least 3, in other embodiments at least 4, in other embodiments all five of the aforementioned proteins.

Reference herein to “secrete”/“secreting”/“secretion” relates to a detectable secretion of the indicated factor, above background levels in standard assays. For example, 0.5×10⁶ fetal or maternal ASC can be suspended in 4 ml medium (DMEM+10% fetal bovine serum (FBS)+2 mM L-Glutamine), added to each well of a 6 well-plate, and cultured for 24 hrs in a humidified incubator (5% CO₂, at 37° C.). After 24 h, DMEM is removed, and cells are cultured for an additional 24 hrs in 1 ml RPMI 1640 medium+2 mM L-Glutamine+0.5% HSA. The CM is collected from the plate, and cell debris is removed by centrifugation.

In certain embodiments, in vitro, the described ASC stimulate endothelial cell proliferation (ECP), or in another embodiment inhibit T cell proliferation, or in another embodiment perform both activities. In certain embodiments, as provided herein, when 750 human umbilical cord endothelial cells (HUVEC) are incubated for 4 days under normoxic conditions at 37° C. on a layer of the ASC in a tissue culture dish, proliferation of the HUVEC cells is at least 120%, at least 125%, at least 130%, at least 140%, at least 150%, and least 160%, or at least 180% of the level observed in the absence of ASC, as provided in International Patent Appl. Pub. No WO2016/098061, which is incorporated herein by reference in its entirety.

In other embodiments, each of CD73, CD29, and CD105 is expressed by more than 90% of the described ASC; and the cells stimulate ECP. In yet other embodiments, each of CD34, CD19, and CD14 is expressed by less than 3% of the cells; and the cells stimulate ECP. In other embodiments, each of CD73, CD29, and CD105 is expressed by more than 90% of the cells, each of CD34, CD19, and CD14 is expressed by less than 3% of the cells; and the cells stimulate ECP.

Alternatively or in addition, the ASC secrete a factor(s) that promotes angiogenesis. In certain embodiments, the ASC secrete a factor selected from VEGF (vascular endothelial growth factor), angiogenin, Angiopoietin 1, MCP-1, IL-8, Serpin E1, and GCP2/CXCL6. In other embodiments, the ASC secrete VEGF, Angiogenin, Angiopoietin 1, MCP-1, IL-8, and Serpin E1, which were found to be secreted by maternal cells. In still other embodiments, the ASC secrete VEGF, Angiogenin, Angiopoietin 1, MCP-1, IL-8, Serpin E1, and GCP2/CXCL6, which were found to be secreted by fetal cells.

In still other embodiments, the ASC secrete Flt-3 ligand (Fms-related tyrosine kinase 3 ligand; Uniprot Accession No. P49772), stem cell factor (SCF; Uniprot Accession No. P21583), IL-6 (Interleukin-6; UniProt identifier P05231), or combinations thereof, each of which represents a separate embodiment. In certain embodiments, the ASC secrete levels of Flt-3 ligand, SCF, IL-6, or in other embodiments combinations thereof, that are at least 2-, 3-, 4-, 5-, 6-, 8-, 10-, 12-, 15-, or 20-fold higher than that expressed or secreted by ASC of placenta tissue grown on a 2D substrate. ASC grown on a 3D substrate secrete higher levels of Flt-3 ligand, SCF, and IL-6 than ASC grown on a 2D substrate, as provided in PCT Application Publ. No. WO/2007/108003, which is fully incorporated herein by reference in its entirety. In certain embodiments, the described ASC are placental ASC. Uniprot entries in this and the following 2 paragraphs were accessed on Feb. 26, 2017.

According to some embodiments, the described ASC are capable of suppressing an immune reaction in the subject.

Methods of determining the immunosuppressive capability of a cell population are known to those skilled in the art, and exemplary methods are described in Example 3 of PCT Publication No. WO 2009/144720, which is incorporated herein by reference in its entirety. For example, a mixed lymphocyte reaction (MLR) may be performed. In an exemplary, non-limiting MLR assay, irradiated cord blood (iCB) cells, for example human cells or cells from another species, are incubated with peripheral blood-derived monocytes (PBMC; for example human PBMC or PBMC from another species), in the presence or absence of a cell population to be tested. PBMC cell replication, which correlates with the intensity of the immune response, can be measured by a variety of methods known in the art, for example by ³H-thymidine uptake. Reduction of the PBMC cell replication when co-incubated with test cells indicates an immunosuppressive capability. Alternatively or in addition, secretion of pro-inflammatory and anti-inflammatory cytokines by blood cell populations (such as monocytes or PBMC) can be measured when stimulated (for example by incubation with non-matched cells, or with a non-specific stimulant such as PHA), in the presence or absence of the ASC. In certain embodiments, for example in the case of human ASC, as provided in WO 2009/144720, which is incorporated herein by reference, when 200,000 PBMC are co-incubated for 48 hours with 4,000 allogeneic ASC, followed by a 5-hour stimulation with 1.5 mcg/ml of LPS, the amount of IL-10 secretion by the PBMC is at least 120%, at least 130%, at least 1500%, at least 170%, at least 200%, or at least 300% of the amount observed with LPS stimulation in the absence of ASC.

In other embodiments, each of CD73, CD29, and CD105 is expressed by more than 90% of the described placental ASC; and the cells inhibit T cell proliferation. In yet other embodiments, each of CD34, CD19, and CD14 is expressed by less than 3% of the cells; and the cells inhibit T cell proliferation. In other embodiments, each of CD73, CD29, and CD105 is expressed by more than 90% of the cells, each of CD34, CD19, and CD14 is expressed by less than 3% of the cells; and the cells inhibit T cell proliferation.

In still other embodiments, the placental ASC secrete immunoregulatory factor(s). In certain embodiments, the ASC secrete Leukemia inhibitory factor (LIF; UniProt identifier P15018). In other embodiments, the ASC secrete a factor selected from MCP-1 (CCL2), Osteoprotegerin, MIF (Macrophage migration inhibitory factor; Uniprot Accession No. P14174), GDF-15, SDF-1 alpha, GROa (Growth-regulated alpha protein; Uniprot Accession No. P09341), beta2-Microglobulin, IL-6, IL-8 (UniProt identifier P10145), ENA78/CXCL5, eotaxin/CCL11 (Uniprot Accession No. P51671), and MCP-3 (CCL7). In certain embodiments, the ASC secrete MCP-1, Osteoprotegerin, MIF, GDF-15, SDF-1 alpha, GROa, beta2-Microglobulin, IL-6, IL-8, and MCP-3, which were found to be secreted by maternal cells. In other embodiments, the ASC secrete MCP-1, Osteoprotegerin, MIF, GDF-15, SDF-1 alpha, beta2-Microglobulin, IL-6, IL-8, ENA78, eotaxin, and MCP-3, which were found to be secreted by fetal cells. All Swissprot and UniProt entries in this paragraph were accessed on Mar. 23, 2017.

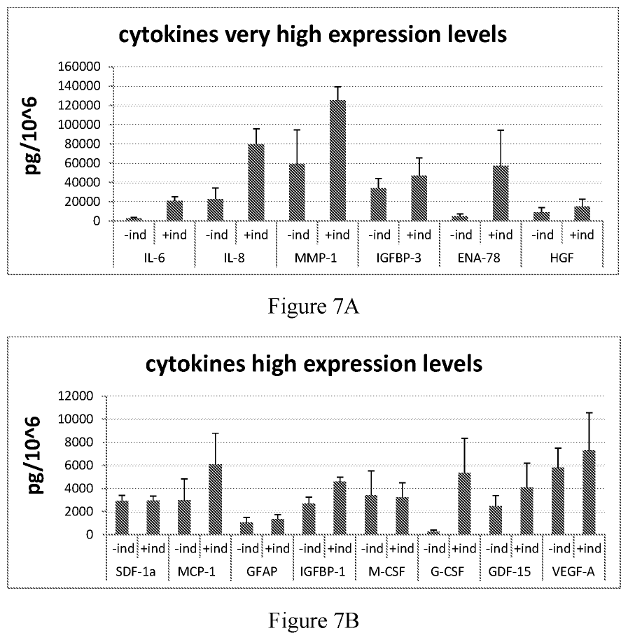

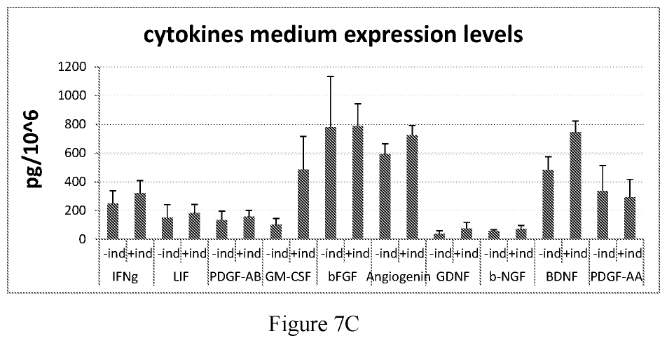

In other embodiments, the described placental ASC, or in other embodiments the MSC, secrete a factor(s) that promotes neurogenesis. In certain embodiments, the ASC secrete BDNF. In more specific embodiments, the ASC secrete over 200 pg/ml BDNF, when 2×10\hat{ }5 cells (following induction and optionally cryopreservation) are seeded in 6-well plates, in 2 ml DMEM+10% FBS medium, followed by incubation in serum-free DMEM for 72 hours and measurement of BDNF in the CM. In other embodiments, under the same conditions, the ASC secrete over 250 pg/ml BDNF; over 300 pg/ml BDNF; over 400 pg/ml BDNF; over 500 pg/ml BDNF; over 600 pg/ml BDNF; over 800 pg/ml BDNF; over 1000 pg/ml BDNF; over 1200 pg/ml BDNF; over 1500 pg/ml BDNF; or over 1800 pg/ml BDNF. In other embodiments, the ASC secrete over 2000 pg/ml BDNF; over 2500 pg/ml BDNF; over 3000 pg/ml BDNF; over 4000 pg/ml BDNF; over 5000 pg/ml BDNF; over 6000 pg/ml BDNF; or over 7000 pg/ml BDNF, when the CM is produced in DMEM+20% FBS (Example 7). In other embodiments, the ASC secrete over 1000; over 1200; over 1500; over 2000; over 2500; over 3000; or over 3500 pg BDNF per 10\hat{ }6 cells into the induction medium itself (Example 7). In other embodiments, the aforementioned amounts of BDNF are secreted in the first, the second, or the third 24-hour period of incubation in serum-free DMEM. In still other embodiments, the ASC secrete elevated amounts of a factor selected from LIF, BDNF, GDNF, VEGF, G-CSF, IL-6, HGF, and IL-8. In still other embodiments, the ASC secrete a cytokine selected from any of the other factors shown in FIG. 7A-C, after directly incubating cryopreserved cells for 24 hr. in DMEM+20% FBS (Example 7). Each factor represents a separate embodiment of the present invention.

In still other embodiments, the placental ASC secrete a factor selected from BDNF (brain derived neurotrophic factor; Uniprot Accession No. P23560), GDNF, bFGF (basic fibroblast growth factor), NGF (beta-nerve growth factor; Uniprot Accession No. P01138), VEGF (vascular endothelial growth factor; Uniprot Accession No. P15692), HGF (hepatocyte growth factor; Uniprot Accession No. P08581), and LIF (Leukemia inhibitory factor; Uniprot Accession No. P15018). In certain embodiments, the secreted factors comprise one or more of BDNF, GDNF, bFGF, NGF, VEGF, and HGF. Uniprot Accession Numbers in this paragraph were accessed on May 22, 2017.

In other embodiments, the described placental ASC exhibit a spindle shape when cultured under 2D conditions.

According to some embodiments, the placental ASC express CD200, while in other embodiments, the ASC lack expression of CD200. In still other embodiments, less than 30%, 25%, 20%, 15%, 10%, 8%, 6%, 5%, 4%, 3%, or 2%, 1%, or 0.5% of the adherent cells express CD200.

In yet other embodiments, greater than 70%, 75%, 80%, 85%, 90%, 92%, 94%, 95%, 96%, 97%, 98%, 99%, or 99.5% of the adherent cells express CD200.

In still other embodiments, the cells may be allogeneic, or in other embodiments, the cells may be autologous. In other embodiments, the cells may be fresh or, in other embodiments, frozen (for example, cryo-preserved).

Additional Method Characteristics for Preparation of ASC

In certain embodiments, the described ASC have been subject to a 3D incubation, as described further herein. In more specific embodiments, the ASC have been incubated in a 2D adherent-cell culture apparatus, prior to the step of 3D culturing. In some embodiments, cells (which have been extracted, in some embodiments, from placenta, or in other embodiments, from BM, from adipose tissue, etc.) are then subjected to prior step of incubation in a 2D adherent-cell culture apparatus, followed by the described 3D culturing steps.

The terms “two-dimensional culture” and “2D culture” refer to a culture in which the cells are exposed to conditions that are compatible with cell growth and allow the cells to grow in a monolayer, which is referred to as a “2D culture apparatus”. Such apparatuses will typically have flat growth surfaces (also referred to as a “two-dimensional substrate(s)” or “2D substrate(s)”), in some embodiments comprising an adherent material, which may be flat or curved. Non-limiting examples of apparatuses for 2D culture are cell culture dishes and plates. Included in this definition are multi-layer trays, such as Cell Factory™, manufactured by Nunc™, provided that each layer supports monolayer culture. It will be appreciated that even in 2D apparatuses, cells can grow over one another when allowed to become over-confluent. This does not affect the classification of the apparatus as “two-dimensional”.

The terms “three-dimensional culture” and “3D culture” refer to a culture in which the cells are exposed to conditions that are compatible with cell growth and allow the cells to grow in a 3D orientation relative to one another. The term “three-dimensional [or 3D] culture apparatus” refers to an apparatus for culturing cells under conditions that are compatible with cell growth and allow the cells to grow in a 3D orientation relative to one another. Such apparatuses will typically have a 3D growth surface (also referred to as a “three-dimensional substrate” or “3D substrate”), in some embodiments comprising an adherent material, which is present in the 3D culture apparatus, e.g. the bioreactor. Certain, non-limiting embodiments of 3D culturing conditions suitable for expansion of adherent stromal cells are described in PCT Application Publ. No. WO/2007/108003, which is fully incorporated herein by reference in its entirety.

In various embodiments, “an adherent material” refers to a material that is synthetic, or in other embodiments naturally occurring, or in other embodiments a combination thereof. In certain embodiments, the material is non-cytotoxic (or, in other embodiments, is biologically compatible). Alternatively or in addition, the material is fibrous, which may be, in more specific embodiments, a woven fibrous matrix, a non-woven fibrous matrix, or any type of fibrous matrix. In still other embodiments, the material exhibits a chemical structure such as charged surface exposed groups, which allows cell adhesion. Non-limiting examples of adherent materials which may be used in accordance with this aspect include a polyester, a polypropylene, a polyalkylene, a polyfluorochloroethylene, a polyvinyl chloride, a polystyrene, a polysulfone, a cellulose acetate, a glass fiber, a ceramic particle, a poly-L-lactic acid, and an inert metal fiber. Other embodiments include Matrigel™, an extra-cellular matrix component (e.g., Fibronectin, Chondronectin, Laminin), and a collagen. In more particular embodiments, the material may be selected from a polyester and a polypropylene. Non-limiting examples of synthetic adherent materials include polyesters, polypropylenes, polyalkylenes, polyfluorochloroethylenes, polyvinyl chlorides, polystyrenes, polysulfones, cellulose acetates, and poly-L-lactic acids, glass fibers, ceramic particles, and an inert metal fiber, or, in more specific embodiments, polyesters, polypropylenes, polyalkylenes, polyfluorochloroethylenes, polyvinyl chlorides, polystyrenes, polysulfones, cellulose acetates, and poly-L-lactic acids.

In other embodiments, the length of 3D culturing is at least 4 days; between 4-12 days; in other embodiments between 4-11 days; in other embodiments between 4-10 days; in other embodiments between 4-9 days; in other embodiments between 5-9 days; in other embodiments between 5-8 days; in other embodiments between 6-8 days; or in other embodiments between 5-7 days. In other embodiments, the 3D culturing is performed for 5-15 cell doublings, in other embodiments 5-14 doublings, in other embodiments 5-13 doublings, in other embodiments 5-12 doublings, in other embodiments 5-11 doublings, in other embodiments 5-10 doublings, in other embodiments 6-15 cell doublings, in other embodiments 6-14 doublings, in other embodiments 6-13 doublings, or in other embodiments 6-12 doublings, in other embodiments 6-11 doublings, or in other embodiments 6-10 doublings.

In certain embodiments, 3D culturing can be performed in a 3D bioreactor. In some embodiments, the 3D bioreactor comprises a container for holding medium and a 3D attachment substrate disposed therein, and a control apparatus, for controlling pH, temperature, and oxygen levels and optionally other parameters. The terms attachment substrate and growth substrate are interchangeable. In certain embodiments, the attachment substrate is in the form of carriers, which comprise, in more specific embodiments, a surface comprising a synthetic adherent material. Alternatively or in addition, the bioreactor contains ports for the inflow and outflow of fresh medium and gases. Except where indicated otherwise, the term “bioreactor” excludes decellularized organs and tissues derived from a living being.

Examples of bioreactors include, but are not limited to, a continuous stirred tank bioreactor, a CelliGen Plus® bioreactor system (New Brunswick Scientific (NBS) and a BIOFLO 310 bioreactor system (New Brunswick Scientific (NBS).

As provided herein, a 3D bioreactor is capable, in certain embodiments, of 3D expansion of ASC under controlled conditions (e.g. pH, temperature and oxygen levels) and with growth medium perfusion, which in some embodiments is constant perfusion and in other embodiments is adjusted in order to maintain target levels of glucose or other components. Furthermore, the cell cultures can be directly monitored for concentrations of glucose, lactate, glutamine, glutamate and ammonium. The glucose consumption rate and the lactate formation rate of the adherent cells enable, in some embodiments, measurement of cell growth rate and determination of the harvest time.

In some embodiments, a continuous stirred tank bioreactor is used, where a culture medium is continuously fed into the bioreactor and a product is continuously drawn out, to maintain a time-constant steady state within the reactor. A stirred tank bioreactor with a fibrous bed basket is available for example from New Brunswick Scientific Co., Edison, N.J.). Additional bioreactors that may be used, in some embodiments, are stationary-bed bioreactors; and air-lift bioreactors, where air is typically fed into the bottom of a central draught tube flowing up while forming bubbles, and disengaging exhaust gas at the top of the column. Additional possibilities are cell-seeding perfusion bioreactors with polyactive foams [as described in Wendt, D. et al., Biotechnol Bioeng 84: 205-214, (2003)] and radial-flow perfusion bioreactors containing tubular poly-L-lactic acid (PLLA) porous scaffolds [as described in Kitagawa et al., Biotechnology and Bioengineering 93(5): 947-954 (2006). Other bioreactors which can be used are described in U.S. Pat. Nos. 6,277,151; 6,197,575; 6,139,578; 6,132,463; 5,902,741; and 5,629,186, which are incorporated herein by reference. A “stationary-bed bioreactor” refers to a bioreactor in which the cellular growth substrate is not ordinarily lifted from the bottom of the incubation vessel in the presence of growth medium. For example, the substrate may have sufficient density to prevent being lifted and/or it may be packed by mechanical pressure to present it from being lifted. The substrate may be either a single body or multiple bodies. Typically, the substrate remains substantially in place during the standard perfusion rate of the bioreactor. In certain embodiments, the substrate may be lifted at unusually fast perfusion rates, for example greater than 200 rpm.

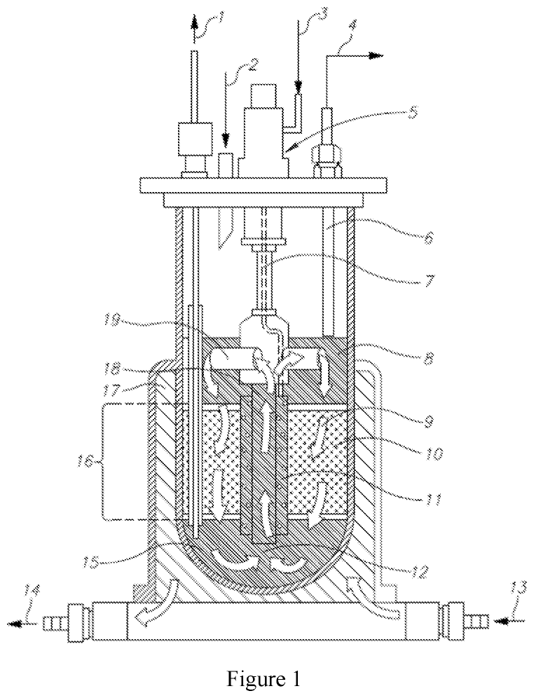

Another exemplary, non-limiting bioreactor, the Celligen 310 Bioreactor, is depicted in FIG. 1. A Fibrous-Bed Basket (16) is loaded with polyester disks (10). In some embodiments, the vessel is filled with deionized water or isotonic buffer via an external port (1 [this port may also be used, in other embodiments, for cell harvesting]) and then optionally autoclaved. In other embodiments, following sterilization, the liquid is replaced with growth medium, which saturates the disk bed as depicted in (9). In still further embodiments, temperature, pH, dissolved oxygen concentration, etc., are set prior to inoculation. In yet further embodiments, a slow stirring initial rate is used to promote cell attachment, then agitation is increased. Alternatively or addition, perfusion is initiated by adding fresh medium via an external port (2). If desired, metabolic products may be harvested from the cell-free medium above the basket (8). In some embodiments, rotation of the impeller creates negative pressure in the draft-tube (18), which pulls cell-free effluent from a reservoir (15) through the draft tube, then through an impeller port (19), thus causing medium to circulate (12) uniformly in a continuous loop. In still further embodiments, adjustment of a tube (6) controls the liquid level; an external opening (4) of this tube is used in some embodiments for harvesting. In other embodiments, a ring sparger (not visible), is located inside the impeller aeration chamber (11), for oxygenating the medium flowing through the impeller, via gases added from an external port (3), which may be kept inside a housing (5), and a sparger line (7). Alternatively or in addition, sparged gas confined to the remote chamber is absorbed by the nutrient medium, which washes over the immobilized cells. In still other embodiments, a water jacket (17) is present, with ports for moving the jacket water in (13) and out (14).

In certain embodiments, a perfused bioreactor is used, wherein the perfusion chamber contains carriers. The carriers may be, in more specific embodiments, selected from macrocarriers, microcarriers, or both together. Non-limiting examples of microcarriers that are available commercially include alginate-based (GEM, Global Cell Solutions), dextran-based (Cytodex, GE Healthcare), collagen-based (Cultispher, Percell), and polystyrene-based (SoloHill Engineering) microcarriers. In certain embodiments, the microcarriers are packed inside the perfused bioreactor.

In some embodiments, the carriers in the perfused bioreactor are packed, for example forming a packed bed, which is submerged in a nutrient medium. Alternatively or in addition, the carriers may comprise an adherent material. In other embodiments, the surface of the carriers comprises an adherent material, or the surface of the carriers is adherent. In still other embodiments, the material exhibits a chemical structure such as charged surface exposed groups, which allows cell adhesion. Non-limiting examples of adherent materials which may be used in accordance with this aspect include a polyester, a polypropylene, a polyalkylene, a polyfluorochloroethylene, a polyvinyl chloride, a polystyrene, a polysulfone, a cellulose acetate, a glass fiber, a ceramic particle, a poly-L-lactic acid, and an inert metal fiber. In more particular embodiments, the material may be selected from a polyester and a polypropylene. In various embodiments, an “adherent material” refers to a material that is synthetic, or in other embodiments naturally occurring, or in other embodiments a combination thereof. In certain embodiments, the material is non-cytotoxic (or, in other embodiments, is biologically compatible). Non-limiting examples of synthetic adherent materials include polyesters, polypropylenes, polyalkylenes, polyfluorochloroethylenes, polyvinyl chlorides, polystyrenes, polysulfones, cellulose acetates, and poly-L-lactic acids, glass fibers, ceramic particles, and an inert metal fiber, or, in more specific embodiments, polyesters, polypropylenes, polyalkylenes, polyfluorochloroethylenes, polyvinyl chlorides, polystyrenes, polysulfones, cellulose acetates, and poly-L-lactic acids. Other embodiments include Matrigel™, an extra-cellular matrix component (e.g., Fibronectin, Chondronectin, Laminin), and a collagen.

In other embodiments, cells are produced using a packed-bed spinner flask. In more specific embodiments, the packed bed may comprise a spinner flask and a magnetic stirrer. The spinner flask may be fitted, in some embodiments, with a packed bed apparatus, which may be, in more specific embodiments, a fibrous matrix; or in more specific embodiments, a non-woven fibrous matrix. In other embodiments, the fibrous matrix comprises polyester, or comprises at least about 50% polyester. In still other embodiments, the non-woven fibrous matrix comprises polyester, or comprises at least about 50% polyester.

In still other embodiments, the matrix is similar to the Celligen™ Plug Flow bioreactor which is, in certain embodiments, packed with Fibra-cel® carriers (or, in other embodiments, other carriers). The spinner is, in certain embodiments, batch fed (or in other alternative embodiments fed by perfusion), fitted with one or more sterilizing filters, and placed in a tissue culture incubator. In further embodiments, cells are seeded onto the scaffold by suspending them in medium and introducing the medium to the apparatus. In still further embodiments, the agitation speed is gradually increased, for example by starting at 40 RPM for 4 hours, then gradually increasing the speed to 120 RPM. In certain embodiments, the glucose level of the medium may be tested periodically (i.e. daily), and the perfusion speed adjusted maintain an acceptable glucose concentration, which is, in certain embodiments, between 400-700 mg\liter, between 450-650 mg\liter, between 475-625 mg\liter, between 500-600 mg\liter, or between 525-575 mg\liter. In yet other embodiments, at the end of the culture process, carriers are removed from the packed bed, washed with isotonic buffer, and processed or removed from the carriers by agitation and/or enzymatic digestion.

In certain embodiments, the bioreactor is seeded at a concentration of between 10,000-2,000,000 cells/ml of medium, in other embodiments 20,000-2,000,000 cells/ml, in other embodiments 30,000-1,500,000 cells/ml, in other embodiments 40,000-1,400,000 cells/ml, in other embodiments 50,000-1,300,000 cells/ml, in other embodiments 60,000-1,200,000 cells/ml, in other embodiments 70,000-1,100,000 cells/ml, in other embodiments 80,000-1,000,000 cells/ml, in other embodiments 80,000-900,000 cells/ml, in other embodiments 80,000-800,000 cells/ml, in other embodiments 80,000-700,000 cells/ml, in other embodiments 80,000-600,000 cells/ml, in other embodiments 80,000-500,000 cells/ml, in other embodiments 80,000-400,000 cells/ml, in other embodiments 90,000-300,000 cells/ml, in other embodiments 90,000-250,000 cells/ml, in other embodiments 90,000-200,000 cells/ml, in other embodiments 100,000-200,000 cells/ml, in other embodiments 110,000-1,900,000 cells/ml, in other embodiments 120,000-1,800,000 cells/ml, in other embodiments 130,000-1,700,000 cells/ml, in other embodiments 140,000-1,600,000 cells/ml.

In still other embodiments, between 1-20×10⁶ cells per gram (gr) of carrier (substrate) are seeded, or in other embodiments 1.5-20×10⁶ cells/gr carrier, or in other embodiments 1.5-18×10⁶, or in other embodiments 1.8-18×10⁶, or in other embodiments 2-18×10⁶, or in other embodiments 3-18×10⁶, or in other embodiments 2.5-15×10⁶, or in other embodiments 3-15×10⁶, or in other embodiments 3-14×10⁶, or in other embodiments 3-12×10, or in other embodiments 3.5-12×10⁶, or in other embodiments 3-10×10⁶, or in other embodiments 3-9×10′, or in other embodiments 4-9×10⁶, or in other embodiments 4-8×10⁶, or in other embodiments 4-7×10⁶, or in other embodiments 4.5-6.5×10⁶ cells/gr carrier.

In certain embodiments, the harvest from the bioreactor is performed when at least about 10%, in other embodiments at least 12%, in other embodiments at least 14%, in other embodiments at least 16%, in other embodiments at least 18%, in other embodiments at least 20%, in other embodiments at least 22%, in other embodiments at least 24%, in other embodiments at least 26%, in other embodiments at least 28%, or in other embodiments at least 30% of the cells are in the S and G2/M phases (collectively), as can be assayed by various methods known in the art, for example FACS detection. Typically, in the case of FACS, the percentage of cells in S and G2/M phase is expressed as the percentage of the live cells, after gating for live cells, for example using a forward scatter/side scatter gate. Those skilled in the art will appreciate that the percentage of cells in these phases correlates with the percentage of proliferating cells. In some cases, allowing the cells to remain in the bioreactor significantly past their logarithmic growth phase causes a reduction in the number of cells that are proliferating.

In other embodiments, over 5×10⁵, over 7×10⁵, over 8×10⁵, over 9×10⁵, over 10⁶, over 1.5×10⁶, over 2×10⁶, over 3×10⁶, over 4×10⁶, or over 5×10⁶ viable cells are removed per milliliter of the growth medium in the bioreactor. In still other embodiments over between 5×10⁵-1.5×10⁷, between 7×10⁵-1.5×10⁷, between 8×10⁵-1.5×10⁷, between 1×10⁶-1.5×10⁷, between 5×10⁵-1×10⁷, between 7×10⁵-1×10⁷, between 8×10⁵-1×10⁷, between 1×10⁶-1×10⁷, between 1.2×10⁶-1×10⁷, or between 2×10⁶-1×10⁷ viable cells are removed per milliliter of the growth medium in the bioreactor.

In other embodiments, incubation of ASC may comprise microcarriers, which may, in certain embodiments, be inside a bioreactor. Microcarriers are known to those skilled in the art, and are described, for example in U.S. Pat. Nos. 8,828,720, 7,531,334, 5,006,467, which are incorporated herein by reference. Microcarriers are also commercially available, for example as Cytodex™ (available from Pharmacia Fine Chemicals, Inc.), Superbeads (commercially available from Flow Labs, Inc.), and DE-52 and DE-53 (commercially available from Whatman, Inc.). In certain embodiments, the ASC may be incubated in a 2D apparatus, for example tissue culture plates or dishes, prior to incubation in microcarriers. In other embodiments, the ASC are not incubated in a 2D apparatus prior to incubation in microcarriers. In certain embodiments, the microcarriers are packed inside a bioreactor.

In some embodiments, as described in WO/2014/037862, published on Mar. 13, 2014, which is incorporated herein by reference in its entirety, grooved carriers are used for proliferation and/or incubation of ASC. In various embodiments, the carriers may be used following a 2D incubation (e.g. on culture plates or dishes), or without a prior 2D incubation. In other embodiments, incubation on the carriers may be followed by incubation on a 3D substrate in a bioreactor, which may be, for example, a packed-bed substrate or microcarriers; or incubation on the carriers may not be followed by incubation on a 3D substrate. Carriers can include multiple two-dimensional (2D) surfaces 12 extending from an exterior of carrier towards an interior of carrier. In certain embodiments, the surfaces are formed by a group of ribs that are spaced apart to form openings, which may be sized to allow flow of cells and culture medium during use. Carriers can also include multiple 2D surfaces extending from a central carrier axis of the carrier and extending generally perpendicular to ribs that are spaced apart to form openings, creating multiple 2D surfaces. In some embodiments, carriers are “3D bodies” as described in WO/2014/037862; the contents of which relating to 3D bodies are incorporated herein by reference.

In certain embodiments, the described carriers (e.g. grooved carriers) are used in a bioreactor. In some, the carriers are in a packed conformation.

In still other embodiments, the material forming the multiple 2D surfaces comprises at least one polymer. Suitable coatings may, in some embodiments, be selected to control cell attachment or parameters of cell biology.

Additional Preparation Steps

In certain embodiments, further steps of purification or enrichment for ASC may be performed. Such methods include, but are not limited to, cell sorting using markers for ASC and/or, in various embodiments, mesenchymal stromal cells or mesenchymal-like ASC.

Cell sorting, in this context, refers to any procedure, whether manual, automated, etc., that selects cells on the basis of their expression of one or more markers, their lack of expression of one or more markers, or a combination thereof. Those skilled in the art will appreciate that data from one or more markers can be used individually or in combination in the sorting process.

In more particular embodiments, cells may be removed from a 3D matrix while the matrix remains within the bioreactor. In certain embodiments, at least about 10%, at least 12%, at least 14%, at least 16%, at least 18%, at least 20%, at least 22%, at least 24%, at least 26%, at least 28%, or at least 30% of the cells are in the S and G2/M phases (collectively), at the time of harvest from the bioreactor. Cell cycle phases can be assayed by various methods known in the art, for example FACS detection. Typically, in the case of FACS, the percentage of cells in S and G2/M phase is expressed as the percentage of the live cells, after gating for live cells, for example using a forward scatter/side scatter gate. Those skilled in the art will appreciate that the percentage of cells in these phases correlates with the percentage of proliferating cells. In some cases, allowing the cells to remain in the bioreactor significantly past their logarithmic growth phase causes a reduction in the number of cells that are proliferating.

In certain embodiments, the harvesting process comprises agitation. In certain embodiments, the agitation is vibration, for example as described in PCT International Application Publ. No. WO 2012/140519, which is incorporated herein by reference. In certain embodiments, during harvesting, the cells are agitated at 0.7-6 Hertz, or in other embodiments 1-3 Hertz, during, or in other embodiments during and after, treatment with a protease, optionally also comprising a calcium chelator. In certain embodiments, the carriers containing the cells are agitated at 0.7-6 Hertz, or in other embodiments 1-3 Hertz, while submerged in a solution or medium comprising a protease, optionally also comprising a calcium chelator. Non-limiting examples of a protease plus a calcium chelator are trypsin, or another enzyme with similar activity, optionally in combination with another enzyme, non-limiting examples of which are Collagenase Types 1, II, III, and IV, with EDTA.

Those skilled in the art will appreciate that a variety of isotonic buffers may be used for washing cells and similar uses. Hank's Balanced Salt Solution (HBSS; Life Technologies) is only one of many buffers that may be used.

Non-limiting examples of base media useful in 2D and 3D culturing include Minimum Essential Medium Eagle, ADC-1, LPM (Bovine Serum Albumin-free), F10 (HAM), F12 (HAM), DCCM1, DCCM2, RPMI 1640, BGJ Medium (with and without Fitton-Jackson Modification), Basal Medium Eagle (BME—with the addition of Earle's salt base), Dulbecco's Modified Eagle Medium (DMEM), Yamane, IMEM-20, Glasgow Modification Eagle Medium (GMEM), Leibovitz L-15 Medium, McCoy's 5A Medium, Medium M199 (M199E—with Earle's sale base), Medium M199 (M199H—with Hank's salt base), Minimum Essential Medium Eagle (MEM-E—with Earle's salt base), Minimum Essential Medium Eagle (MEM-H—with Hank's salt base) and Minimum Essential Medium Eagle (MEM-NAA with non-essential amino acids), among numerous others, including medium 199, CMRL 1415, CMRL 1969, CMRL 1066, NCTC 135, MB 75261, MAB 8713, DM 145, Williams' G, Neuman & Tytell, Higuchi, MCDB 301, MCDB 202, MCDB 501, MCDB 401, MCDB 411, MDBC 153. In certain embodiments, DMEM is used. These and other useful media are available from GIBCO, Grand Island, N.Y., USA and Biological Industries, Bet HaEmek, Israel, among others.

In some embodiments, the medium may be supplemented with additional substances. Non-limiting examples of such substances are serum, which is, in some embodiments, fetal serum of cows or other species, which is, in some embodiments, 5-15% of the medium volume. In certain embodiments, the medium contains 1-5%, 2-5%, 3-5%, 1-10%, 2-10%, 3-10%, 4-15%, 5-14%, 6-14%, 6-13%, 7-13%, 8-12%, 8-13%, 9-12%, 9-11%, or 9.5%-10.5% serum, which may be fetal bovine serum, or in other embodiments another animal serum. In still other embodiments, the medium is serum-free.

Alternatively or in addition, the medium may be supplemented by growth factors, vitamins (e.g. ascorbic acid), cytokines, salts (e.g. B-glycerophosphate), steroids (e.g. dexamethasone) and hormones e.g., growth hormone, erythropoietin, thrombopoietin, interleukin 3, interleukin 7, macrophage colony stimulating factor, c-kit ligand/stem cell factor, osteoprotegerin ligand, insulin, insulin-like growth factor, epidermal growth factor, fibroblast growth factor, nerve growth factor, ciliary neurotrophic factor, platelet-derived growth factor, and bone morphogenetic protein.

It will be appreciated that additional components may be added to the culture medium. Such components may be antibiotics, antimycotics, albumin, amino acids, and other components known to the art for the culture of cells.

The various media described herein, i.e. the 2D growth medium and the 3D growth medium, may be independently selected from each of the described embodiments relating to medium composition. In various embodiments, any medium suitable for growth of cells in a standard tissue apparatus and/or a bioreactor may be used.

It will also be appreciated that in certain embodiments, when the described ASC are intended for administration to a human subject, the cells and the culture medium (e.g., with the above-described medium additives) are substantially xeno-free, i.e., devoid of any animal contaminants e.g., mycoplasma. For example, the culture medium can be supplemented with a serum-replacement, human serum and/or synthetic or recombinantly produced factors.

In other embodiments, conditioned medium (CM) derived from the described ASC is utilized in the described methods, for example post-incubation medium from the described tissue culture incubation or bioreactor incubation. In yet other embodiments, there is provided a pharmaceutical composition comprising the CM, which may be, in some embodiments, indicated for the described therapeutic indications. Those skilled in the art will appreciate that, in certain embodiments, various bioreactors may be used to prepare CM, including but not limited to plug-flow bioreactors, and stationary-bed bioreactors (Kompier R et al. Use of a stationary bed reactor and serum-free medium for the production of recombinant proteins in insect cells. Enzyme Microb Technol. 1991. 13(10):822-7.) For example, CM is produced as a by-product of the described methods for cell expansion. The CM in the bioreactor can be removed from the bioreactor or otherwise isolated. In other embodiments, the described expanded cells are removed from the bioreactor and incubated in another apparatus (a non-limiting example of which is a tissue culture apparatus), and CM from the cells is collected.

In yet other embodiments, extracellular vesicles, e.g. exosomes, secreted by the described ASC are used in the described methods and compositions. Methods of isolating exosomes are known in the art, and include, for example, immuno-magnetic isolation, for example as described in Clayton A et al, 2001; Mathias R A et al, 2009; and Crescitelli R et al, 2013.

In some embodiments, the exosomes or other extracellular vesicles are harvested from a 3D bioreactor in which the ASC have been incubated. Alternatively or in addition, the cells are cryopreserved, and then are thawed, after which the exosomes are isolated. In some embodiments, after thawing, the cells are cultured in 2D culture, from which the exosomes are harvested.

Pharmaceutical Compositions

The described ASC, or CM derived therefrom, can be administered as a part of a pharmaceutical composition, e.g., that further comprises one or more pharmaceutically acceptable carriers. Hereinafter, the term “pharmaceutically acceptable carrier” refers to a carrier or a diluent. In some embodiments, a pharmaceutically acceptable carrier does not cause significant irritation to a subject. In some embodiments, a pharmaceutically acceptable carrier does not abrogate the biological activity and properties of administered cells. Examples, without limitations, of carriers are propylene glycol, saline, emulsions and mixtures of organic solvents with water. In some embodiments, the pharmaceutical carrier is an aqueous solution of saline.

In other embodiments, compositions are provided herein that comprise ASC or CM in combination with an excipient, e.g., a pharmacologically acceptable excipient. In further embodiments, the excipient is an osmoprotectant or cryoprotectant, an agent that protects cells from the damaging effect of freezing and ice formation, which may in some embodiments be a permeating compound, non-limiting examples of which are dimethyl sulfoxide (DMSO), glycerol, ethylene glycol, formamide, propanediol, poly-ethylene glycol, acetamide, propylene glycol, and adonitol, or may in other embodiments be a non-permeating compound, non-limiting examples of which are lactose, raffinose, sucrose, trehalose, and d-mannitol. In other embodiments, both a permeating cryoprotectant and a non-permeating cryoprotectant are present. In other embodiments, the excipient is a carrier protein, a non-limiting example of which is albumin. In still other embodiments, both an osmoprotectant and a carrier protein are present; in certain embodiments, the osmoprotectant and carrier protein may be the same compound. Alternatively or in addition, the composition is frozen. The cells may be any embodiment of ASC mentioned herein, each of which is considered a separate embodiment.

Provided in addition are pharmaceutical compositions, comprising the described placental ASC, in the absence of non-placental cell types.

Also provided are pharmaceutical compositions, comprising the described placental ASC-derived CM, in the absence of CM derived from other cell types.

In other embodiments, there are provided pharmaceutical compositions, comprising the described exosomes.

Since non-autologous cells may in some cases induce an immune reaction when administered to a subject, several approaches may be utilized according to the methods provided herein to reduce the likelihood of rejection of non-autologous cells. In some embodiments, these approaches include either suppressing the recipient immune system or encapsulating the non-autologous cells in immune-isolating, semipermeable membranes before transplantation. In some embodiments, this may be done regardless of whether the ASC themselves engraft in the host. For example, the majority of the cells may, in various embodiments, not survive after engraftment for more than 3 days, more than 4 days, more than 5 days, more than 6 days, more than 7 days, more than 8 days, more than 9 days, more than 10 days, or more than 14 days. In other embodiments, an immunosuppressive agent is present in the pharmaceutical composition.

One may, in various embodiments, administer the pharmaceutical composition in a systemic manner (as detailed hereinabove). Alternatively, one may administer the pharmaceutical composition locally, for example, via injection of the pharmaceutical composition directly into an exposed or affected tissue region of a patient. In other embodiments, the cells are administered intravenously (IV), subcutaneously (SC), by the intraosseous route (e.g. by intraosseous infusion), or intraperitoneally (IP), each of which is considered a separate embodiment. In other embodiments, the ASC or composition is administered intramuscularly; while in other embodiments, the ASC or composition is administered systemically. In this regard, “intramuscular” administration refers to administration into the muscle tissue of a subject; “subcutaneous” administration refers to administration just below the skin; “intravenous” administration refers to administration into a vein of a subject; “intraosseous” administration refers to administration directly into bone marrow; and “intraperitoneal” administration refers to administration into the peritoneum of a subject. In other embodiments, the cells are administered intracerebroventricularly. In certain embodiments, lung-targeting routes of administration may utilize cells encapsulated in liposomes or other barriers to reduce entrapment within the lungs.

In still other embodiments, the cells are administered intranasally. Intranasal delivery devices are known in the art. In certain embodiments, the device is Sipnose, or in other embodiments, is another device described in US Patent Appl. Publ. No. 2017/0128678 (Daniel Shahaf et al.), which is incorporated herein by reference in its entirety.

In other embodiments, for injection, the described cells may be formulated in aqueous solutions, e.g. in physiologically compatible buffers such as Hank's solution, Ringer's solution, or physiological salt buffer, optionally in combination with medium containing cryopreservation agents.

For any preparation used in the described methods, the therapeutically effective amount or dose can be estimated initially from in vitro and cell culture assays. Often, a dose is formulated in an animal model to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals.

A typical systemic dosage of the described ASC for a human subject ranges, in some embodiments, from 10-500 million cells per administration. For example, the dosage can be, in some embodiments, 10, 20, 30, 40, 50, 60, 70, 80, 90, 100, 125, 150, 175, 200, 225, 250, 275, 300, 325, 350, 375, 400, 425, 450, 475, or 500 million cells or any amount in between these numbers. A typical intracranial dose for a human subject ranges, in some embodiments, from 10-200 million, 10-150 million, 20-200 million, 20-150 million, 10-100 million, 10-80 million, 20-100 million, 20-80 million, 30-100 million, or 30-80 million cells per administration. A typical intranasal dose for a human subject ranges, in some embodiments, from 10-300 million, 10-200 million, 10-150 million, 20-300 million, 20-200 million, 20-150 million, 10-100 million, 10-80 million, 20-100 million, 20-80 million, 30-100 million, or 30-80 million cells per administration. ASC, compositions comprising ASC, and/or medicaments manufactured using ASC can be administered, in various embodiments, in a series of 1, 2, 3, 4, 5, 6, 7, 8, 9, 10, 11, 12, 13, 14, 15, 1-10, 1-15, 1-20, 2-10, 2-15, 2-20, 3-20, 4-20, 5-20, 5-25, 5-30, 5-40, or 5-50 injections. In still other embodiments, the ASC are administered periodically, e.g. weekly or monthly, until drug cravings subside.

Depending on the severity and responsiveness of the condition to be treated, dosing can be of a single or, in other embodiments, a plurality of administrations, with a course of treatment lasting from several days to several weeks or, in other embodiments, until alleviation of the disease state is achieved.

In certain embodiments, following administration, the majority of the cells, in other embodiments more than 60%, more than 70%, more than 80%, more than 90%, more than 95%, more than 96%, more than 97%, more than 98%, or more than 99% of the cells are no longer detectable within the subject 1 month after administration.

Compositions including the described preparations formulated in a compatible pharmaceutical carrier may also be prepared, placed in an appropriate container, and labeled for treatment of an indicated condition.

The described compositions may, if desired, be packaged in a container that is accompanied by instructions for administration. The container may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

In other embodiments, the described ASC are suitably formulated as a pharmaceutical composition which can be suitably packaged as an article of manufacture. Such an article of manufacture comprises a packaging material which comprises a label describing a use in treating a disease or disorder or therapeutic indication that is mentioned herein. In other embodiments, a pharmaceutical agent is contained within the packaging material, wherein the pharmaceutical agent is effective for the treatment of a disorder or therapeutic indication that is mentioned herein. In some embodiments, the pharmaceutical composition is frozen.

It is clarified that each embodiment of the described ASC may be freely combined with each embodiment relating to a therapeutic method or pharmaceutical composition.

Furthermore, each embodiment of the described exosomes may be freely combined with each embodiment relating to a therapeutic method or pharmaceutical composition.

In still other embodiments, the described CM is used in any of the described therapeutic methods. Each embodiment of CM may be freely combined with each embodiment relating to a therapeutic method or pharmaceutical composition.

Subjects

In certain embodiments, the subject treated by the described methods and compositions is a human. In some embodiments, the subject is addicted to a drug, e.g. a psychostimulant, or another drug mentioned herein.

Also disclosed herein are kits and articles of manufacture that are drawn to reagents that can be used in practicing the methods disclosed herein. The kits and articles of manufacture can include any reagent or combination of reagent discussed herein or that would be understood to be required or beneficial in the practice of the disclosed methods, including ASC. In another aspect, the kits and articles of manufacture may comprise a label, instructions, and packaging material, for example for treating a disorder or therapeutic indication mentioned herein.

Additional objects, advantages, and novel features of the invention will become apparent to one ordinarily skilled in the art upon examination of the following examples, which are not intended to be limiting. Additionally, each of the various embodiments and aspects of the invention as delineated hereinabove and as claimed in the claims section below finds experimental support in the following examples.

### EXAMPLES

Reference is now made to the following examples, which together with the above descriptions illustrate certain embodiments in a non-limiting fashion.

### Example 1: Culturing and Production of Adherent Placental Cells

Placenta-derived cell populations containing over 90% maternally-derived cells were cultured in 2D culture, followed by bioreactor culture on fibrous carriers, as described in Example 1 of International Patent Application WO 2016/098061, in the name of Esther Lukasiewicz Hagai et al, published on Jun. 23, 2016, which is incorporated herein by reference in its entirety.

To detach the cells from the carriers, carriers were incubated with trypsin solution for 4 minutes, with oscillating mixing at 5 Hz, as described in PCT International Application Publ. No. WO 2012/140519. The medium was drained into a harvest bag, containing FBS (final concentration 10%), and the carriers were washed with isotonic solution, with oscillating mixing at 5-Hz frequency, and the cell suspension was drained into the harvest bag. Cells were then suspended and washed in suspension solution (5% w/v human serum albumin [HSA] in isotonic solution), then adjusted to 10-20×10⁶ cells/ml, in isotonic solution with 10% DMSO v/v and 5% HSA w/v. The vials were gradually chilled and stored in a gas-phase liquid nitrogen freezer.

### Example 2: Cell Stock Production in Serum-Free Medium

Methods

The procedure included periodic testing of the medium for sterility and contamination.

### Step 1-1—Extraction and Plating of Adherent Stromal Cells (ASC's)

Placentas were obtained from donors up to 35 years old, who were pre-screened and determined to be negative for hepatitis B, hepatitis C, HIV-1 and HIV-2, HTLV-1 and HTLV-2, and syphilis. The donor placenta was maintained sterile and cooled.

Within 36 hours of the delivery, the placenta (apart from the amnion and chorion) was placed with the maternal side facing upwards and minced. Pieces were washed with isotonic buffer+gentamicin, then incubated for 1-3 hours with collagenase and DNAse in isotonic buffer. DMEM with 10% filtered FBS, L-Glutamine, and gentamicin was added, and cells were filtered through a sterile stainless steel sieve and centrifuged. The cells were suspended in culture medium, seeded in flasks, and incubated at 37° C. in a humidified tissue culture incubator with 5% CO₂.

After 2 days, cells were washed with PBS, and CellStart™ cell attachment solution and StemPro® MSC SFM XenoFree medium (serum-free and xeno-free culture medium [SFM-XF]) (ThermoFisher Scientific, catalog no. A10675-01; hereinafter “StemPro® medium”) were added.

### Step 1-2—Initial Culturing

Cells were cultured for 2 additional passages (typically 4-10 population doublings after the first passage) in StemPro® medium+CellStart™. When reaching 60-90% confluence, cells were detached using trypsin, centrifuged, and seeded at 3.16±0.5×10³ cells/cm² in tissue culture flasks.

### Step 1-3—Cell Concentration, Washing, Formulation, Filling and Cryopreservation

The cell suspension from the final passage was centrifuged and suspended in culture medium at 20-40×10⁶ cells/milliliter (mL), then adjusted to 10% DMSO, 400% FBS, and 50% DMEM, the temperature was reduced in a controlled rate freezer, and cells were stored in a liquid nitrogen freezer to produce the Intermediate Cell Stock (“ICS”).

Results

Cell characteristics of several batches were assessed (Table 1).

TABLE 1
Characteristics of placental cells expanded in SF medium.
PDL refers to population doubling level-in this case,
the number of doublings since passage 1.

| | | | Total growth | cell size | |
|---|---|---|---|---|---|
| BATCH | GROUP | Passage | (days) | (μm) | PDL |
| PD200114SFM | A | 1 | 8 | 20.3 | NA |
| | | 2 | 14 | 20.9 | 3.4 |
| | | 3 | 20 | 19.7 | 7 |
| | B | 1 | 8 | 19.5 | NA |
| | | 2 | 15 | 21.5 | 3.4 |
| | | 3 | 20 | 18.9 | 6.9 |
| PD240214SFM | A | 1 | 7 | 16.2 | NA |
| | | 2 | 14 | 20.8 | 2.7 |
| | | 3 | 20 | 19.4 | 6.4 |
| | B | 1 | 7 | 22 | NA |
| | | 2 | 14 | 18.2 | 2.1 |
| | | 3 | 20 | 19.2 | 6.1 |
| PD230414SFM | NA | 1 | 7 | NA | NA |
| | | 2 | 14 | NA | 2.3 |
| | | 3 | 19 | 16.2 | 5.7 |
| PD040514SFM | NA | 1 | 7 | NA | NA |
| | | 2 | 14 | NA | 2.7 |
| | | 3 | 18 | 15.6 | 6.5 |
| PD260514SFM | NA | 1 | 7 | NA | NA |
| | | 2 | 13 | NA | 2.9 |
| | | 3 | 17 | 15.8 | 6.6 |
| PD180814SFM | NA | 1 | 6 | NA | NA |
| | | 2 | 10 | NA | 2.1 |
| | | 3 | 16 | 16.7 | 5.3 |
| PD220914SFM | unfiltered | 1 | 8 | NA | NA |
| | | 2 | 14 | NA | 2.1 |
| | | 3 | 20 | 17 | 5.6 |
| | filtered | 1 | 8 | NA | NA |
| | | 2 | 14 | NA | 2 |
| | | 3 | 20 | 17.8 | 5.1 |
| PD271014SFM | filtered | 1 | 9 | NA | NA |
| | | 2 | 15 | NA | 2.1 |
| | | 3 | 21 | 17 | 5.1 |

| | | | |
|---|---|---|---|
| Average P 3 | 19.1 | 17.55 | 6.12 |
| % CV P 3 | 8 | 9 | 11 |

### Example 3: Additional Culturing Steps

### Step 2-1: Additional Two-Dimensional (2D) Cell Culturing.

The ICS was thawed, diluted with and cultured in StemPro® medium until 60-90% confluence (typically 4-7 days after seeding), and cultured for 2 additional passages (referred to as passages 3/1 and 3/2 respectively; again passaging when reaching 60-90% confluence), then were harvested for seeding in the bioreactor.

### Step 2-2: Three Dimensional (3D) Cell Growth in Bioreactor/s

Each bioreactor contained Fibra-cel® carriers (New Brunswick Scientific) made of polyester and polypropylene, and StemPro® medium. The culture medium in the bioreactor/s was kept at the following conditions: temp: 37±1C, Dissolved Oxygen (DO): 70±20% and pH 7.4±0.4. Filtered gases (Air, CO₂, N₂ and O₂) were supplied as determined by the control system in order to maintain the target DO and pH values. After seeding, the medium was stirred with stepwise increases in the speed, up to 150-200 RPM by 24 hours. Perfusion was initiated several hours after seeding and was adjusted on a daily basis in order to keep the glucose concentration constant at approximately 550 mg\liter. Cells were typically harvested after 5-6 days by washing the cells, adding trypsin, and subjecting them to agitation.

### Step 2-3: Downstream Steps: Concentration, Washing, Formulation, and Cryopreservation

Cells were suspended and washed in suspension solution (5% w/v human serum albumin [HSA] in isotonic solution), then adjusted to 10-20×10⁶ cells/ml, in isotonic solution with 10% DMSO v/v and 5% HSA w/v. The vials were gradually chilled and stored in a gas-phase liquid nitrogen freezer.

### Example 4: Osteocyte and Adipocyte Differentiation Assays

ASC were prepared as described in Example 1. BM adherent cells were obtained as described in WO 2016/098061 to Esther Lukasiewicz Hagai and Rachel Ofir, which is incorporated herein by reference in its entirety. Osteogenesis and adipogenesis assays were performed as described in WO 2016/098061.

Osteocyte Induction.

Incubation of BM-derived adherent cells in osteogenic induction medium resulted in differentiation of over 50% of the BM cells, as demonstrated by positive alizarin red staining. On the contrary, none of the placental-derived cells exhibited signs of osteogenic differentiation.

Next, a modified osteogenic medium comprising Vitamin D and higher concentrations of dexamethasone was used. Over 50% of the BM cells underwent differentiation into osteocytes, while none of the placental-derived cells exhibited signs of osteogenic differentiation.

Adipocyte Induction.

Adipocyte differentiation of placenta- or BM-derived adherent cells in adipocyte induction medium resulted in differentiation of over 50% of the BM-derived cells, as demonstrated by positive oil red staining and by typical morphological changes (e.g. accumulation of oil droplets in the cytoplasm). In contrast, none of the placental-derived cells differentiated into adipocytes.

Next, a modified medium containing a higher indomethacin concentration was used. Over 50% of the BM-derived cells underwent differentiation into adipocytes. In contrast, none of the placental-derived cells exhibited morphological changes typical of adipocytes.

### Example 5: Further Osteocyte and Adipocyte Differentiation Assays

ASC were prepared as described in Example 2. Adipogenesis and Osteogenesis were assessed using the STEMPRO® Adipogenesis Differentiation Kit (GIBCO, Cat# A1007001) and the STEMPRO® Osteogenesis Differentiation Kit (GIBCO, Cat# A1007201), respectively.

Results

Adipogenesis and Osteogenesis of placental cells grown in SRM or in full DMEM were tested. Groups are shown in Table 2.

TABLE 2
experimental groups

| Group | Product | Batch |
|---|---|---|
| A1 | BM derived MSC | BM-122 |
| | (positive control) | |
| B1 | ASC grown in SRM | PD220914SFMS3 R001 B1.2 |
| C1 | ASC grown in SRM | R050115 R01 |
| D1 | ASC grown in SRM | R280115 R01 |
| E1 | ASC grown in full DMEM | PT041011R36 |

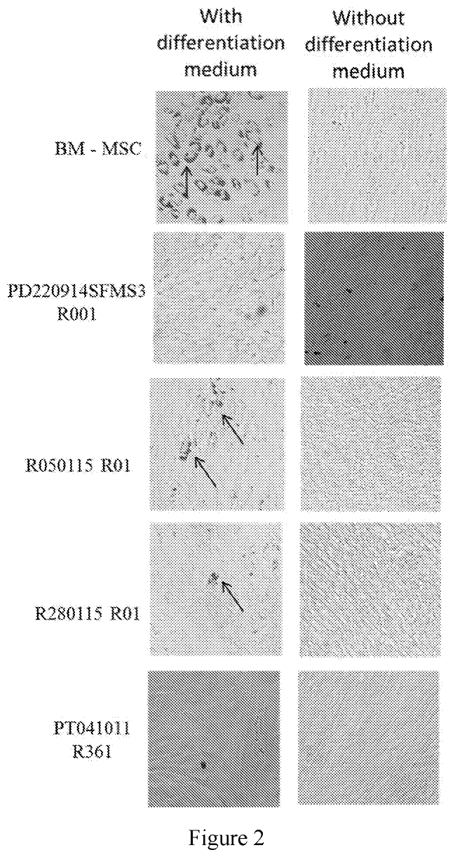

In adipogenesis assays, BM-MSCs treated with differentiation medium stained positively with Oil Red O (FIG. 2). By contrast, 2/3 of the SRM batches exhibited negligible staining, and the other SRM batch, as well as the full DMEM-grown cells, did not exhibit any staining at all, showing that they lacked adipogenic potential.

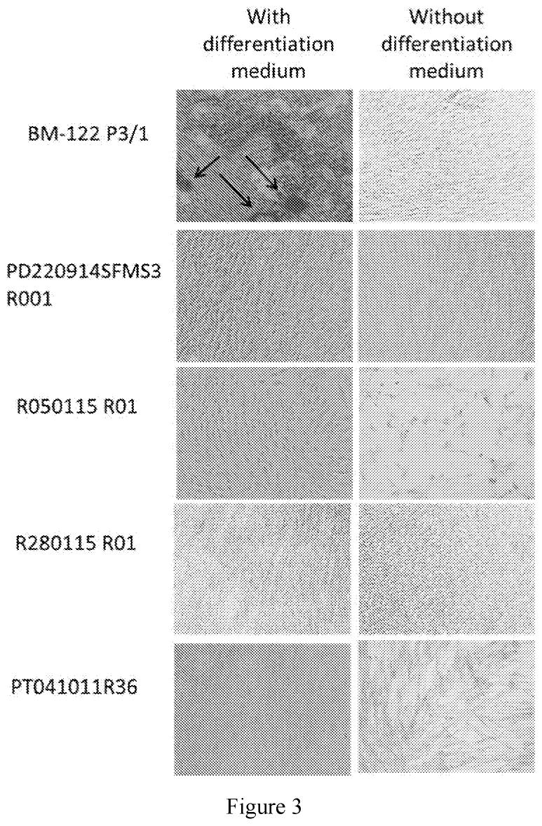

In osteogenesis assays, BM-MSCs treated with differentiation medium stained positively with Alizarin Red S (FIG. 3). By contrast, none of the placental cells batches grown in SRM or full DMEM exhibited staining, showing that they lacked osteogenic potential.

### Example 6: Induction of ASC to Secrete Neurotrophic Factors and Immunomodulatory Cytokines

Methods

At passage 3/3 (the third passage after thawing and plating the ICS [Step 2-1]), 2×10\hat{ }5 placenta-derived cells, primarily of fetal origin, were diluted in DMEM+10% FBS, centrifuged, and suspended and seeded in 6-well plates, in 2 ml DMEM+10% FBS medium per well. After 1 day, the medium was aspirated, the cells were washed in PBS; and incubated in serum-free DMEM supplemented with various combinations of erythropoietin, dibutyryl cyclic AMP (dbcAMP), basic fibroblast growth factor (bFGF), heparin, 3-Isobutyl-1-methylxanthine (IBMX), PDGF-AA (platelet-derived growth factor), neuregulin-1 beta (HRG-beta 1) (Uniprot Accession. No. Q7RTW4), epidermal growth factor (EGF), and 1× N-2 animal-free cell culture supplement (ThermoFisher Scientific, Cat. #1752048) was added. 100× N-2 contains 1 mM human transferrin (holo), 500 mg/L (milligrams per liter) Insulin Recombinant Full Chain, 0.63 mg/L progesterone, 10 mM putrescine, and 0.52 mg/L selenite. The cells were incubated in the supplemented DMEM for 72 hours, after which the conditioned medium (CM) from the cells was collected. The cells were then trypsinized and collected separately.

Analysis of BDNF concentration was performed using either an ELISA kit or a Luminex® kit.

Results

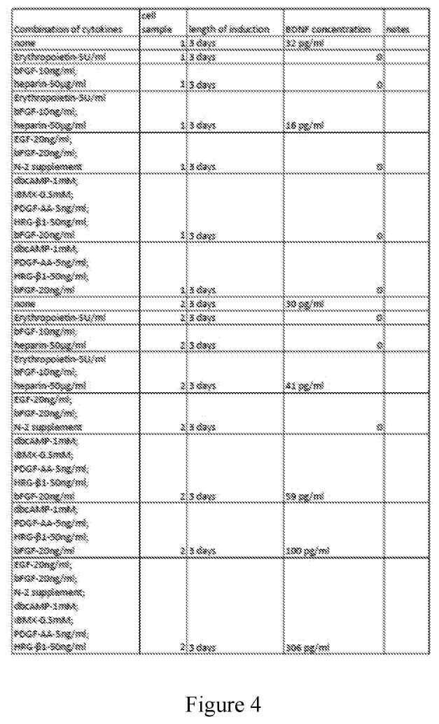

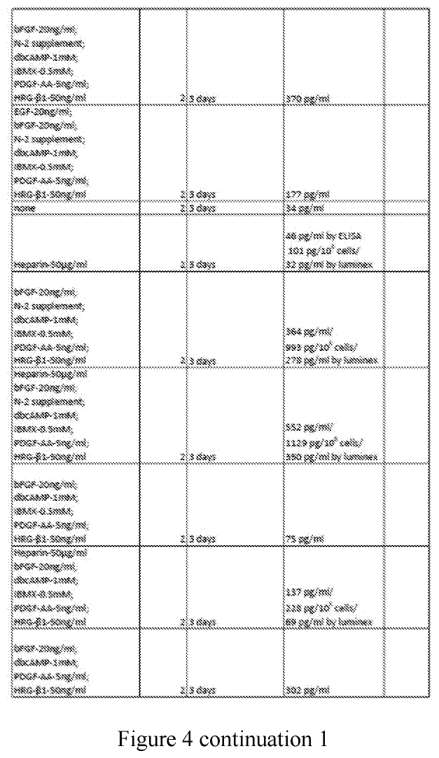

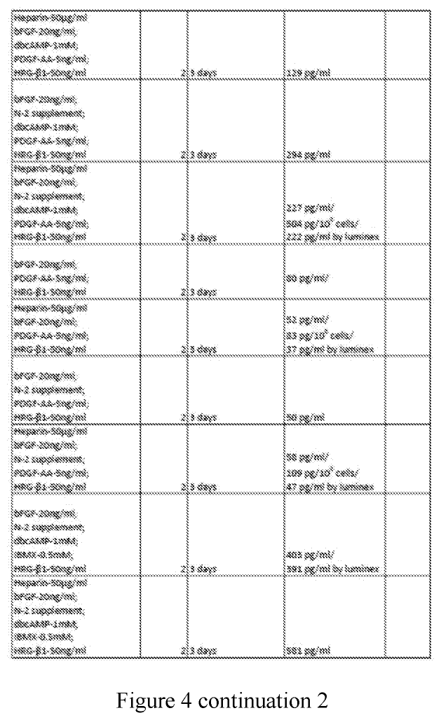

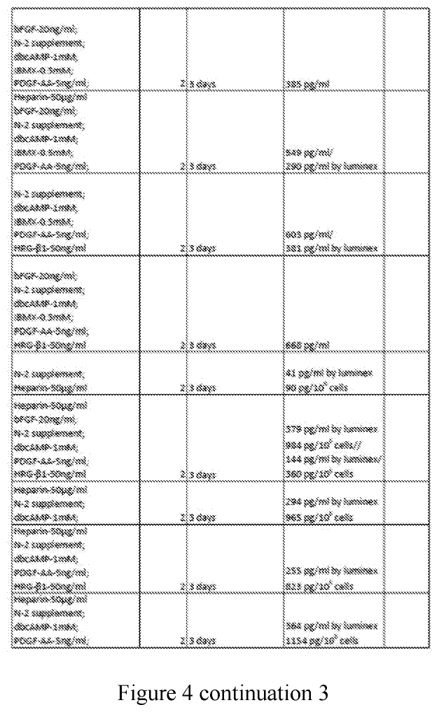

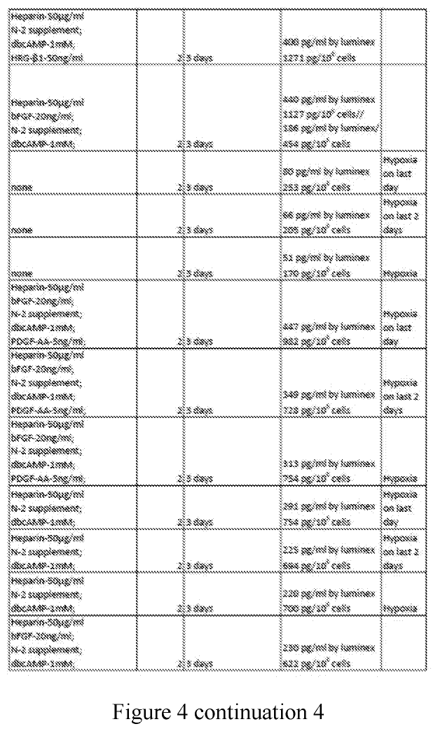

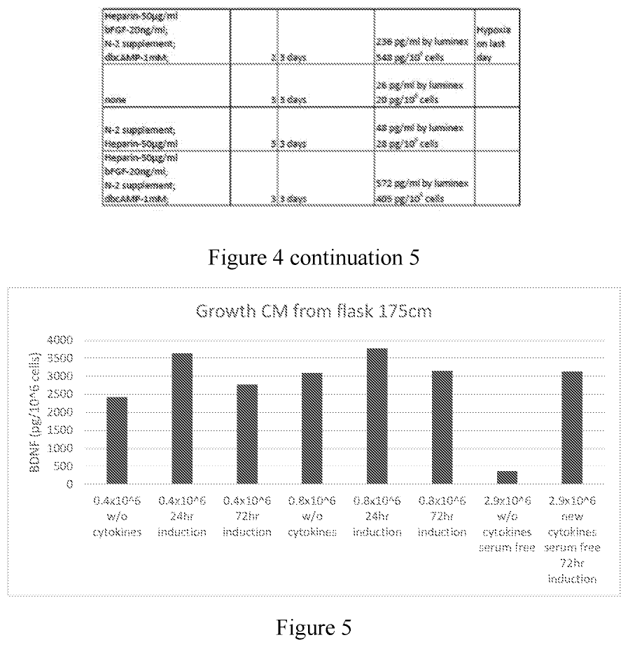

ASC were incubated in DMEM without serum supplemented with various combinations of erythropoietin, dbcAMP, bFGF, heparin, IBMX, PDGF, HRG-beta 1, EGF, and 1× N-2 supplement, in order to define the additives needed for induction. The CM was collected and analyzed for BDNF concentration (FIG. 4). Medium containing dbcAMP, heparin and N-2 supplement achieved induction after an incubation of only 3 days. Inclusion of bFGF further enhanced BDNF secretion and cell viability.

### Example 7: Additional ASC Induction Protocols

As described in the previous Example, thawed cells were seeded in plates, in full DMEM medium, then the next day were washed, and incubated in DMEM supplemented with 1 mM (millimolar) dbcAMP, 20 ng/ml (nanograms per milliliter) bFGF, 50 mcg/ml (microgram/milliliter) heparin, and N-2 supplement to IX concentration. To determine the best conditions for induction, ASC were thawed at p3/3 and were seeded in 175 cm² flasks and grown for 5 days in DMEM+20% FBS, in some cases in the presence of induction agents for the last 24 or 72 hours (induction agents were added either in the presence of DMEM+20% FBS or DMEM without FBS). Induction was similar to Example 6, but with various parameters altered. The initial seeding density was varied, and CM was collected after no induction or 24- or 72-hr. of induction (see Table 3) and tested for BDNF concentration.

Additionally, aliquots of cells were cryopreserved at the conclusion of the induction and were subsequently thawed, seeded in 6-well plates at 0.7×10\hat{ }6 cells/well, and incubated for 72 hr. in DMEM+20% FBS, withdrawing a sample of CM every 24 hrs, which was tested for BDNF concentration. At 72 hours, cells were removed from the plates and counted for normalization.

TABLE 3
Induction Conditions for Samples

| | Cell no./cm\hat{ }2 at | Induction | Agents | |
|---|---|---|---|---|
| Group | time of seeding | time (hr) | present? | Medium |
| 1 | 2300 = | (none) | − | DMEM + 20% |
| 2 | 0.4 × 10\hat{ }6 cells | 24 | + | FBS |
| 3 | | 72 | + | |
| 4 | 4600 = | (none) | − | |
| 5 | 0.8 × 10\hat{ }6 cells | 24 | + | |
| 6 | | 72 | + | |
| 7 | 17,000 = | (none) | − | Basal DMEM |
| 8 | 2.9 × 10\hat{ }6 cells | 72 | + | |
| | (previous | | | |
| | conditions) | | | |

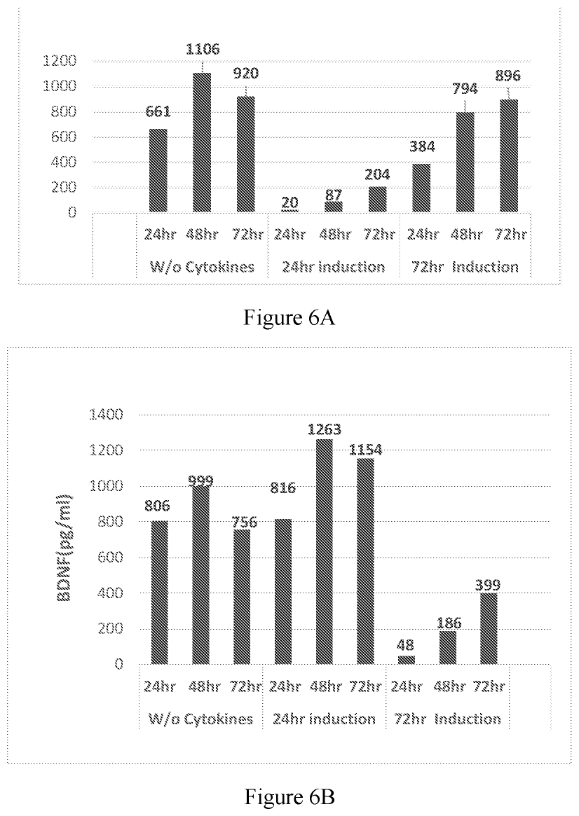

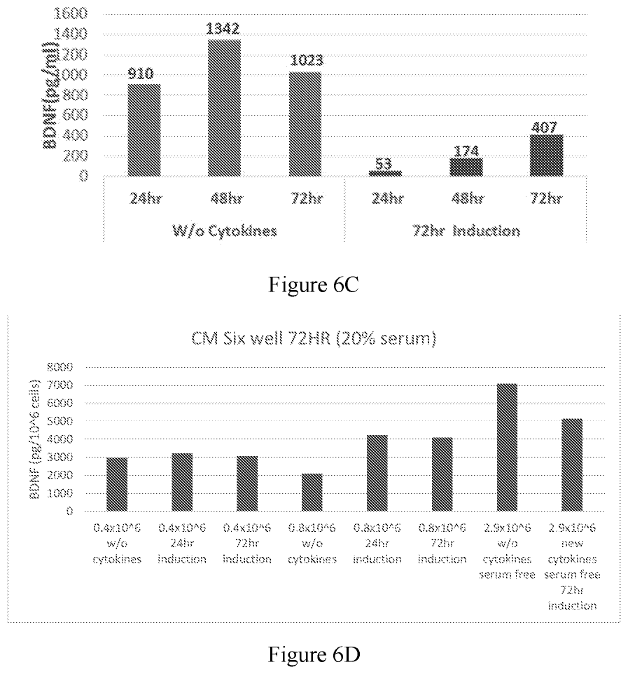

Measurement of BDNF concentration in CM at the conclusion of the induction showed that seeding density did not affect BDNF secretion, and neither did induction agents, unless serum-free conditions were used (FIG. 5; rightmost 2 bars depict serum-free conditions in the absence of presence of induction agents). Aliquots of cells were also cryopreserved after induction, and BDNF secretion was measured after thawing, which is shown for the low-, medium-, and high density groups (FIGS. 6A-C, respectively). BDNF secretion appeared lower after 72-hr. induction in the high-density/serum free group compared to 24 hr. induction in the medium-density group when looking at absolute numbers (compare FIG. 6C, rightmost 3 bars to FIG. 6B, middle set of 3 bars). However, when numbers were normalized to the number of cells, BDNF levels in the high-density/serum free group were similar to the low and medium density groups (FIG. 6D; compare the 1^{st} and 4^{th} bars from the right). This was due to the low cell viability in this group. This showed that use of serum-containing medium for induction enabled cells to better recuperate from cryopreservation and thus the number of viable cells secreting BDNF is higher, hence the higher absolute concentrations observed.

In further experiments, 0.8×10\hat{ }6 cells per 175 cm² flask were seeded and induced with bFGF and cAMP for 24 hr. in DMEM+20% FBS. Post-cryopreservation and thawing, CM was collected after incubating cells for 24 hr. in DMEM w/o FBS (FIGS. 7A-C).

Conditions of 4600 cells/cm\hat{ }2 and 24-hr. induction time were used for further experiments.

### Example 8: Neuronal Differentiation by CM Derived from ASC Bioreactor Expanded or Incubated with Inducing Agents; Induced ASC Reduce ROS Production and Resultant Cell Mortality

Methods

Placental ASC were expanded as described in Example 2 until passage 3/3. ASC were then incubated for 5 days in DMEM+20% FBS, followed by an incubation in DMEM+20% FBS for 24 hr. in the presence of no induction agents or regular concentrations of induction agents, followed by cell cryopreservation and CM collection after cell thawing as described above, but in SH-SY5Y growth medium (MEM/F12) with 10% FBS for the last 24 hr. (CM collected from induced ASC in the absence of serum yielded similar although less pronounced neuronal differentiation).

CM was collected from cryopreserved/thawed bioreactor-expanded ASC as described in the previous Example. SH-SY5Y cells were incubated with regular SH-SY5Y growth medium (composed of 50% MEM medium supplemented with non-essential amino acids; 50% F-12; 10% FBS; 1% glutamine; 0.5% sodium pyruvate and 50 μg/ml gentamycin), CM from induced ASC (prepared in SH-SY5Y growth medium), or regular SH-SY5Y growth medium supplemented with butyric acid or cAMP, to elicit differentiation for 6 days. Medium was replaced after 3 days.

IHC Staining.

Cells were then fixed, permeabilized, and stained with antibodies against human βIII-tubulin and human tyrosine hydroxylase (Abcam), followed by fluorescent labeled secondary antibodies (either Alexa Fluor®488 for tyrosine hydroxylase or CF™543 for βIII tubulin). Nuclei were stained with DAPI. Samples were viewed with an Olympus BX53 fluorescent microscope and the CellSens program was used to take pictures, and analyze neurite length.

Results

CM from induced ASC and bioreactor-expanded ASC, subsequently incubated either with or without induction agents, was incubated with SH-SY5Y cells to determine its ability to induce neuronal differentiation. Certain batches of bioreactor-expanded ASC induced neuronal differentiation indicated by upregulation of beta-III-tubulin expression, a mature neuronal marker, and neurite elongation. High TH and ChAT expression were induced, indicating differentiation of neuronal precursor cells into dopaminergic and cholinergic or noradrenergic neurons.

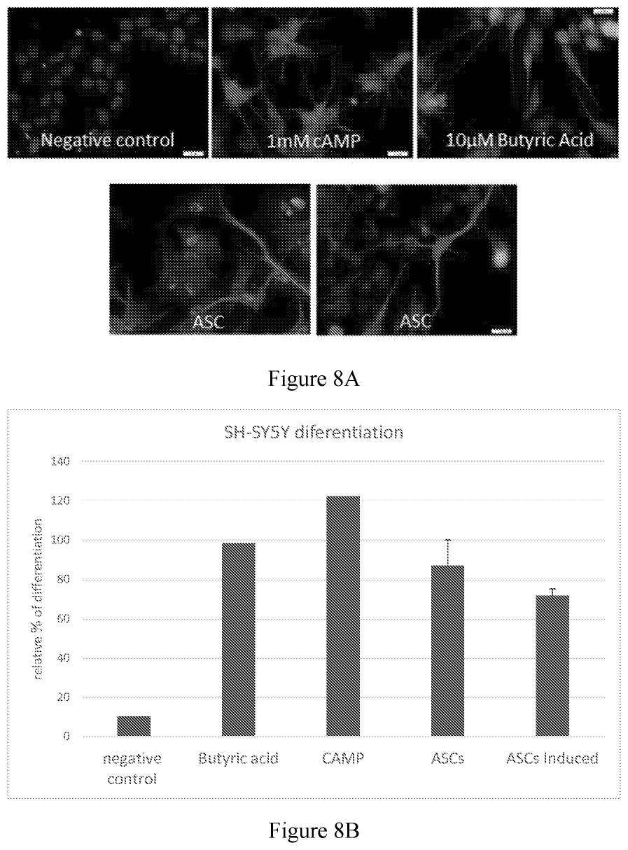

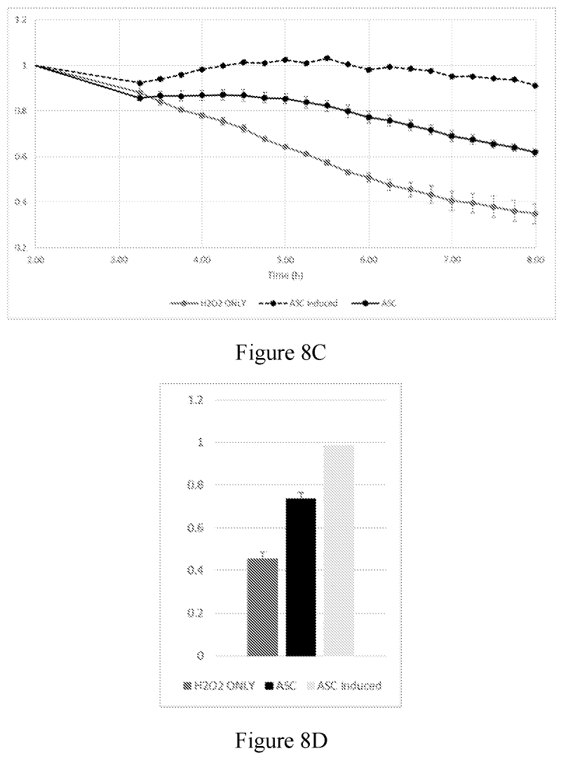

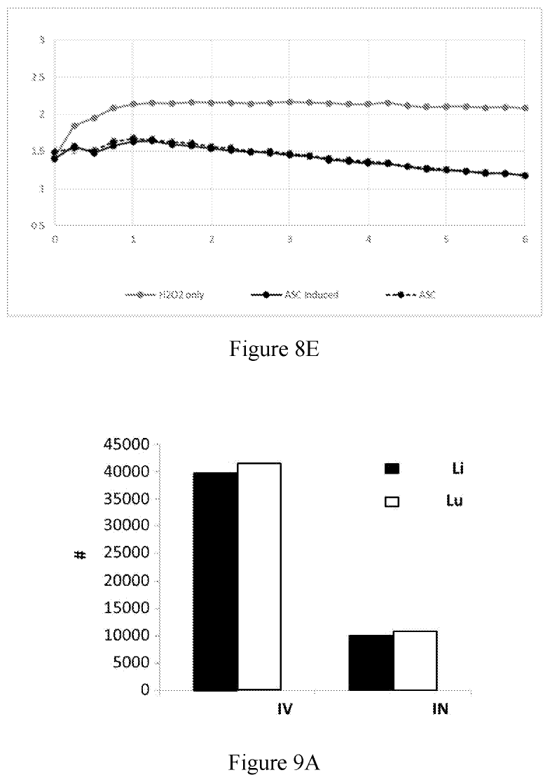

Additionally, CM from certain batches of ASC, whether or not incubated with induction agents, caused neuronal differentiation, as evidenced by an increase in tubulin expression. CM from ASC incubated with induction agents also caused reduced Nestin expression and increased TH and ChAT expression, indicating differentiation of neuronal precursor cells into dopaminergic and cholinergic neurons (FIG. 8A). FIG. 8B shows the quantitation of neuronal differentiation, calculated as the percent of cells with extended neurites that are stained for beta-III-tubulin relative to the retinoic acid positive control.

To determine the cytoprotective/antioxidant effects of placental ASC subjected to bioreactor expansion or incubation with inducing agents, Neuroblastoma (SH-SY5Y) cells were differentiated using cAMP for 7 days, to recapitulate the effect of oxidative stress on fully differentiated neurons in vivo. Following neuronal differentiation, cells were incubated in regular growth medium (control), or CM derived from placental ASC subjected to bioreactor expansion or incubation with inducing agents, in the presence of RealTime GLO™ (RTG) reagents, which detect viable cells. The cells were incubated with the RTG reagents for 2 hours, enabling the RTG to enter the cells and equilibrate. Then H₂O₂ was added to the cells, and luminescence values, correlating with live cell number, were measured every 15 minutes for 8 hours. Cells exposed to H₂O₂ in control medium (solid, gray line) exhibited increased cell death vs. controls without H₂O₂, while CM from placental bioreactor-expanded ASC (solid, black line) or, even more so, following incubation with bFGF and cAMP (dotted line) conferred a significantly higher cell viability (FIG. 8C). The peak difference was seen 6.5 hours following addition of H₂O₂ (FIG. 8D).

Furthermore, formation of ROS was measured using dichlorofluorescin diacetate (DCFDA), a fluorogenic dye that measures intracellular ROS activity within the cell. DCFDA was added to differentiated SH-SY5Y cells for 45 minutes, residual dye was washed away, then CM or control medium (groups labeled as in FIG. 8C) with H₂O₂ were added to the cells. ROS activity was determined every 15 minutes for 6 hours. H₂O₂ caused an increase in ROS activity, as expected, but CM inhibited ROS formation (FIG. 8E). The 2 types of CM behaved similarly to one another.

### Example 9: Localization of ASC after I.N. and I.V. Administration

Methods

Gold nanoparticles (GNPs) are described in Betzer et al.

Cell Uploading of ASC with GNPs:

ASC were suspended in saline (5*10⁵ cells/ml). GNPs were added (2×10 particles per cell), incubated with cells (37° C., 1 hr), and then washed.

Tracking of Migration and Localization of ASC:

Rats were injected either i.n. (5×10⁵ cells) or IV (1×10⁶ cells) with GNP-stained ASC. 24 hours after injection, rats were sacrificed, and the whole body was scanned for GNP stained cells using a microCT imager (Skyscan 1176, Bruker micro-CT, Belgium).

Rat BM-MSC Isolation:

MSC were isolated from freshly-sacrificed male Sprague Dawley (SD) rats by removing tibias and femurs were and cleaning them of connective tissue. Marrow was flushed out of the cut bones after removal of the epiphysis and suspended in DMEM (Biological Industries, Beit Haemek, Israel) with 10% FBS (Biological Industries), 100 units/ml of penicillin, 100 mg/ml of streptomycin, and 2 mM of L-glutamine. Marrow cells were separated and suspended by repeated passage through 19, 20, 21, 23 and 25-G syringe needles. Suspended cells were plated in 100 mm² dishes and cultured at 37° C. and 5% CO². The non-adherent cells were removed at 24 and 48 hours after plating. MSC were expanded in culture for 3-8 passages, changing the medium twice per week. MSC were confirmed to undergo osteogenic differentiation, adipogenic differentiation, and chondrogenic differentiation under standard conditions, and neuronal differentiation, as described below.

Localization:

Studies were performed to quantify the cells that reached each brain region in cocaine-addicted rats (the protocol is described in Example 11). Rats were administered GNP-stained maternal/placental ASC by the i.n. and i.c.v. routes. 28 days later, rat brains were excised, sectioned and desired areas punched out. The amount of GNPs in each area was analyzed using Flame Atomic Absorption Spectroscopy (FAAS) (method described in Betzer et al).

Results

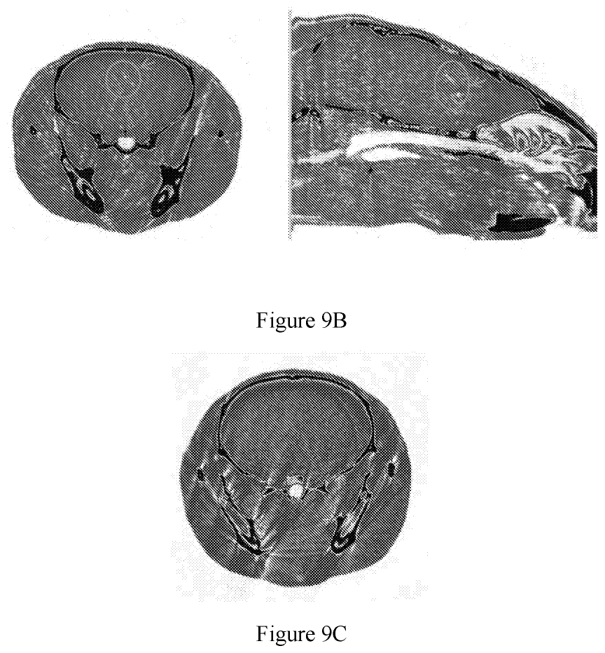

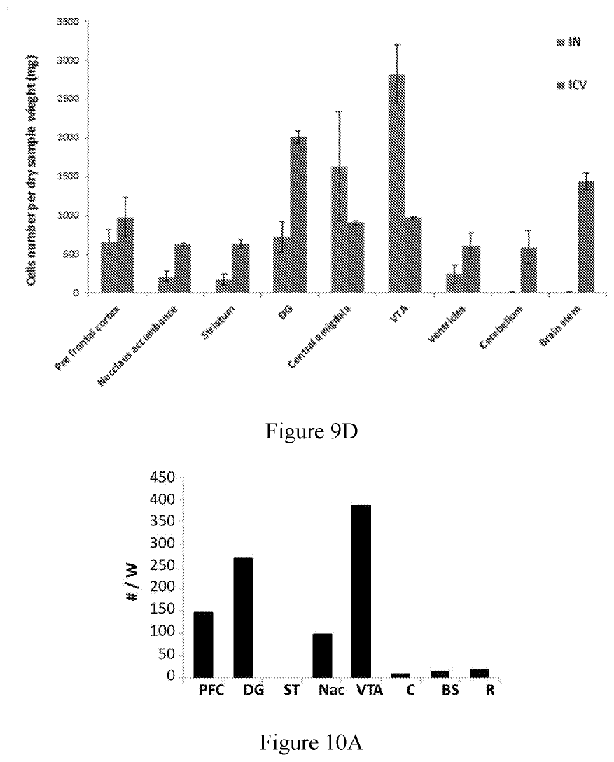

Placental/maternal ASC can be stained with gold nanoparticles (GNPs) (Betzer et al) with only minimal effects on cell viability (as indicated by the percentage of plastic-adherent cells within 6 hours of incubation) and no reduction of cell functionality (as indicated by endothelial cell proliferation and bone marrow migration). Rats received either an i.n. or i.v. injection of placental ASC. CT scanning showed 4-fold lower accumulation of ASC in peripheral organs (liver and lung) after i.n. injection as compared to i.v. injection. Similar results were obtained with rat BM-MSC (FIG. 9A).

Twenty four hours after injection, CT imaging detected a larger number of ASC in the brains of i.n.-injected animals (˜5% of injected cells), relative to i.v. injection. GNPs that were not incubated with cells served as a negative control, since GNPs alone cannot cross the blood-brain barrier under the conditions utilized (FIGS. 9B-C). Placental ASC were found in various brain regions known to be related to drug addiction and reward mechanisms, e.g. the ventral tegmental area (VTA), nucleus accumbens, central amygdala, and dentate gyrus (DG) in brains removed from cocaine addicted placenta ASC-treated rats—28 days after cells' administration (FIG. 9D).

### Example 10: Localization of ASC after I.N. and I.C.V. Administration; ICV Administered MSCs can Induce Neurogenesis

Studies were performed similar to imaging studies in the previous Example, but with non-addicted rats.

Results

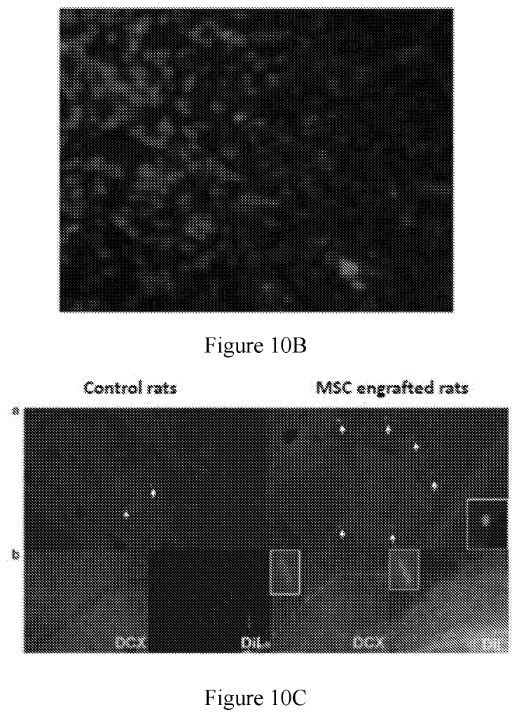

I.n.-administered BM-MSC were found in various brain regions known to be related to drug addiction and reward mechanisms, e.g. the ventral tegmental area (VTA), nucleus accumbens, central amygdala, and dentate gyrus (DG) (FIG. 10A). These results and those of the previous Example show that intranasal injection is a viable route for administration of ASC to areas of the brain involved in drug addiction.

DiI-stained MSC were visualized in the DG by fluorescence (B), following MSC injection into the left lateral ventricle 1 month after administration. Furthermore, enhanced hippocampal neuronal proliferation, as shown by an increase in PCNA-positive cells (indicated by arrows) in the ipsilateral vs. contralateral HC was also observed (C). Immunofluorescence detection of doublecortin (DCX [Uniprot Accession. No. O43602])-positive cells revealed abundant DCX-positive cells (which are dividing neuronal precursor cells) in the granular cell layer of the ipsilateral DG vs. the contralateral DG (magnification×200).

### Example 11: ASC Ameliorate Drug Withdrawal

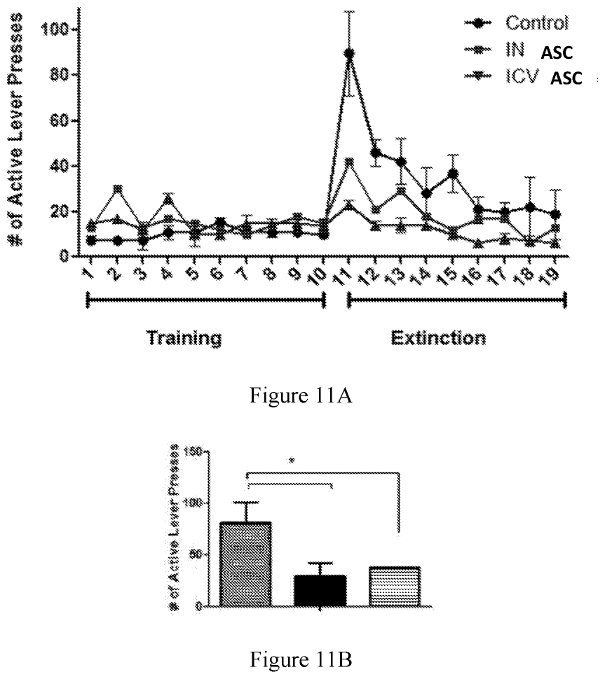

Three groups of rats (n=7 per group) were trained to self-administer cocaine for 1 hour daily for 10 days (until maintenance addiction levels were achieved). Lever presses caused a 20-second i.v. infusion into the jugular vein of (1.5 mg/kg cocaine in 0.13 mL) (obtained from the National Institutes on Drug Abuse, Research Technology Branch, Rockville, Md., USA) under a fixed-ratio-1 schedule of reinforcement. During the 20-s cocaine infusion, active lever presses were recorded but no additional cocaine reinforcement was provided. Rats were then divided into different treatment groups that were randomized to have the same mean drug intake. Different groups received gold nanoparticle-stained placental/maternal ASC, given intranasally (i.n.) or intracerebroventricularly (i.c.v), or vehicle (given i.c.v. or i.n.; average of the 2 groups is depicted) on the first day of the extinction period (which lasted from days 10-19). After the extinction session, rats were subjected to cocaine-primed reinstatement. Craving, as measured by the number of active lever presses, was reduced by both i.c.v. and i.n. ASC during the extinction period (FIG. 11A) and also after cocaine-primed reinstatement two and four weeks following cell administration (FIGS. 11B-C).

### Example 12: Additional Drug Withdrawal Studies with Fetal and Maternal Cells

Methods

Cocaine self-administration: Rats were divided into the following groups: 1. Sham-operated animals—no cocaine exposure, no treatment. 2. Cocaine-addicted, placebo treated. 3. Cocaine-addicted, treated with maternal/placental ASC; 4. Cocaine-addicted, treated with fetal/placental ASC.

Rats were anesthetized with xylazine and ketamine, and a 20-gauge guide cannula was implanted into the lateral ventricle (anterior −0.8, lateral 1.5, ventral −4.0 mm from bregma), and a catheter in the jugular vein. Following 7-day recovery from surgery, accompanied by prophylactic antibiotic injection into the catheter, rats underwent training sessions for 12 study days (until addiction was established) with either cocaine (NIDA, Research Technology Branch, MD) (1 hr./day, 1.5 mg/kg, 0.13 ml, 5 sec/infusion; FR-1; during dark cycle) or saline self-administration (sham). The first day of cocaine exposure was designated as day 0, after which subsequent study day numbers were assigned only on days when animals received training sessions (excluding Saturdays). ASC or placebo (control) was administered i.c.v. between days 12-13 (no training was performed on that day). After reaching maintenance, rats underwent an extinction session from study days 13-23 (inclusive), followed by cocaine-primed reinstatement (10 mg/kg administered IP) and sacrifice on study day 24.

Quantitative Real-Time Polymerase Chain Reaction (qRT-PCR):

Total RNA preparation: miRNeasy Micro Kit (Qiagen, Chatsworth, Calif., USA) was used for purification of miRNA and total RNA from tissues. The quantity and quality of RNA were assessed by NanoDrop 8000 Spectrophotometer (Thermo Scientific, Wilmington, Del., USA) and Agilent 2100 Bioanalyzer following the RNA 6000 Nano Kit protocol (Agilent Technologies, Santa Clara, Calif., USA). RNA samples were stored at −80° C. until use, after which they were reverse-transcribed to cDNAs (qScript cDNA Synthesis, Quanta BioSciences). qRT-PCR analysis was carried out on a Step One Plus Real-time PCR system (ThermoFisher Scientific) using fluorescent SYBR Green FastMix technology (qScript cDNA Synthesis, Quanta BioSciences). Reaction protocols were as follows: 3 seconds at 95° C. for enzyme activation followed by 45 cycles of 5 seconds at 95° C. and 30 seconds at 60° C. Melting curve analysis examined specificity of the amplification products. The primers used for the qRT-PCR analysis are listed in Table 4.

TABLE 4
BDNF primers.

| Gene | Forward primer | Reverse primer |
|---|---|---|
| name | 5′→3′; SEQ No. | 5′→3′; SEQ No. |
| BDNF | gcggcagataaaaag | gccagccaattctat |
| | actgc; 1 | tttg; 2 |

Results

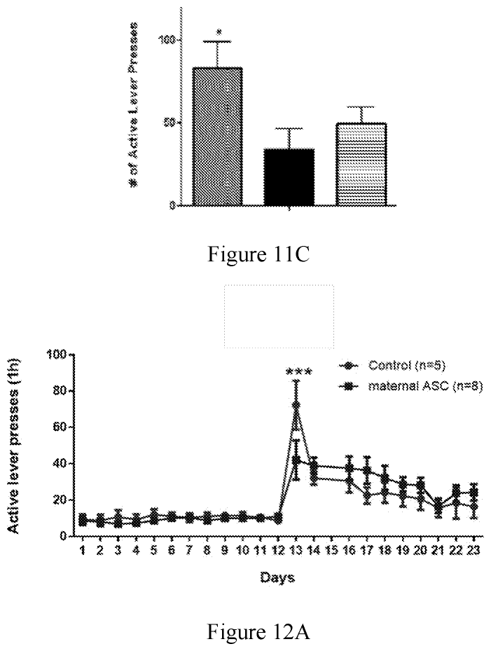

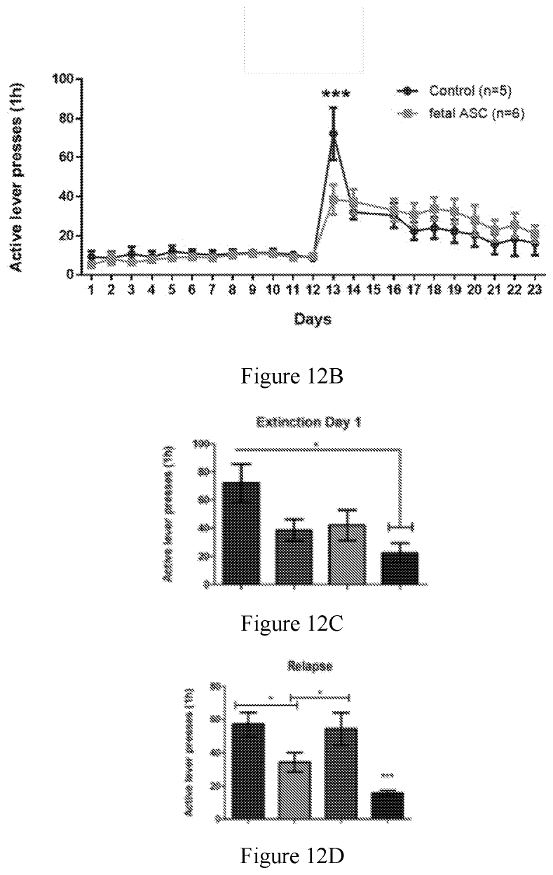

An additional addiction study was performed, with unstained maternal and fetal cells. Rats were trained to self-administer cocaine via a catheter until addiction was established, followed by an extinction period, which was in turn followed by cocaine-primed reinstatement. Rats were divided into different treatment groups, which received either maternal ASC, fetal ASC, or vehicle. An additional control group (“sham operated”) received no cocaine. Both the maternal and fetal ASC reduced peak drug cravings during the first day of extinction (day 13), and maternal ASC demonstrated additional efficacy during induction of relapse (day 24) (FIG. 12A-D).

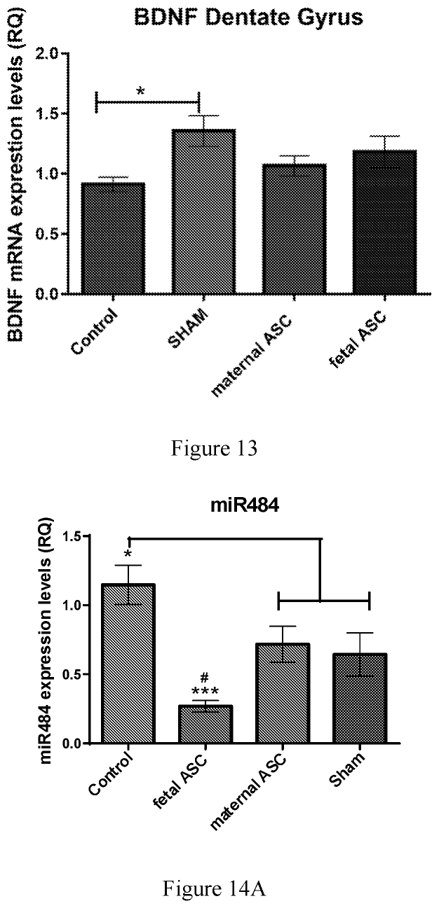

The mRNA expression of brain-derived neurotrophic factor (BDNF) in BM-derived MSCs was assessed using real-time PCR Maternal and fetal ASC also increased levels of BDNF mRNA in the DG (FIG. 13).

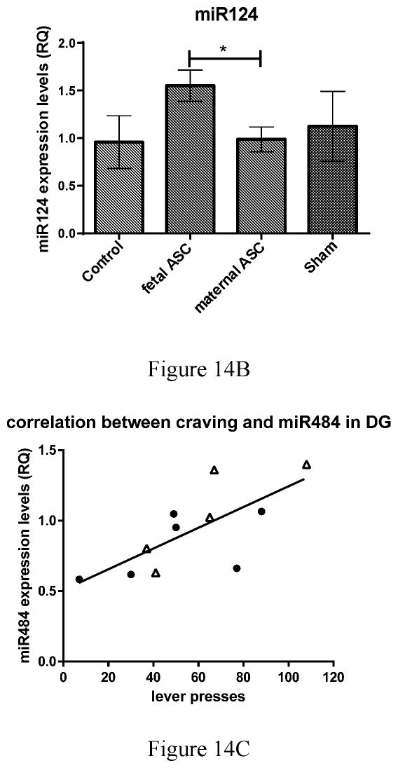

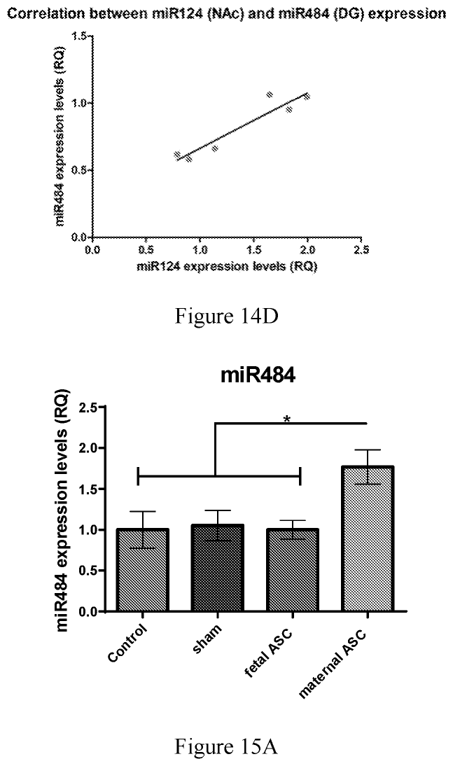

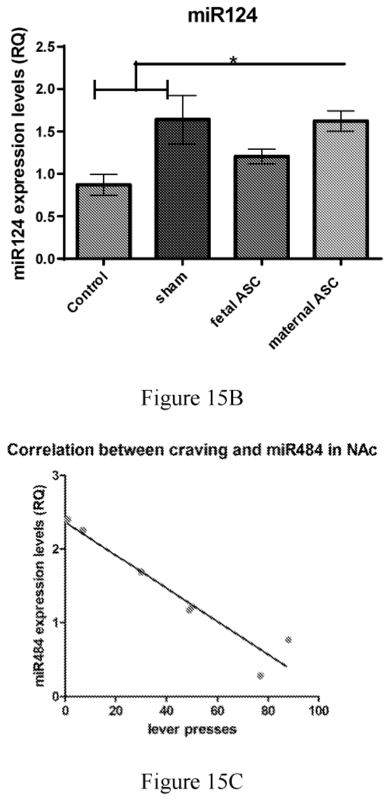

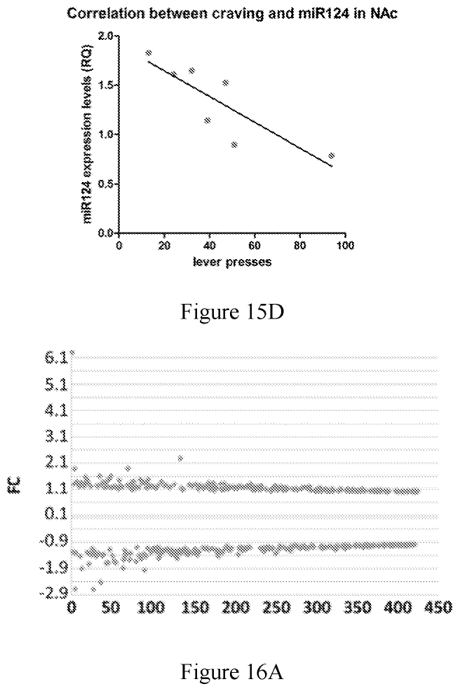

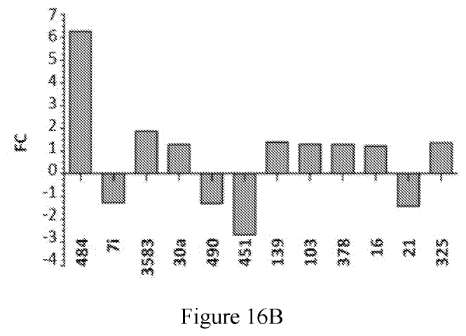

Moreover, maternal and fetal ASC reduced levels of miR484, and fetal ASCs increased miR124 levels in the dentate gyrus (DG), relative to the no-ASC control rats (FIGS. 14A-B). Notably, levels following ASC treatment were similar to levels in non-addicted sham treated animals. miR484 levels positively correlated with craving levels (C; P=0.0119; r=0.7230). In maternal ASC-treated rats, levels of miR124 in the NAc and miR484 in the DG positivity correlated to one another (D). In the NAc, maternal ASC increased levels of miR484, and both ASC types increased miR124 levels (FIGS. 15A-B), and levels of both miRNA's negatively correlated with craving levels (C-D). BM-MSC-treatment of rats also resulted in changes in miRNA (miR) expression in the VTA. FIG. 16A is a scatter plot depicting the differences in expression profile between cocaine addicted rats and sham operated unaddicted rats of 423 miRNAs obtained by Nanostring nCounter analysis system. B shows 12 miRNAs from those presented in A, which showed a statistically significant change (p<0.05) in expression levels after MSC treatment.

### Example 13: Additional Drug Withdrawal Studies with in-Administered ASC

Overall Design:

Sprague-Dawley rats are implanted with a catheter to the jugular vein and allowed to recover, as described for the previous Example. Rats are trained to self-administer cocaine (1.5 mg/Kg) or saline for 1 hour daily, until stable maintenance levels are attained (about 14 days). Rats are then intranasally (i.n.) administered maternal or fetal placental ASC. One day after ASC administration, BrdU is injected i.p. (50 mg/kg, 3 times at 4-h intervals). 2 days after ASC administration, rats undergo an extinction session, followed by cocaine-primed reinstatement (10 mg/kg administered IP), from days 28-44. Addiction levels are determined at several time points by measuring the number of presses of the active (cocaine-supplying) and inactive levers daily during the 7-14 days of extinction. Cocaine priming-induced relapse is measured 28 days after ASC administration. Immediately after the relapse session, rats are perfused, and brains are sectioned and DG area stained for BrdU & NeuN protein (a marker for adult neurons). Sham-operated rats not exposed to cocaine (but rather to saline) and not treated with ASC serve as an additional control group. Different brain areas will also be stained for activated microglia to determine levels of neuronal inflammation.

ASC Administration:

24 hours after concluding training, rats are randomly divided into 3 groups that have the same mean drug intake. Animals are anesthetized with xylazine and ketamine, and maternal or fetal ASC (5×10⁵ in 20-25 μl) or vehicle (25 μl) are infused into each naris (total of 1×10⁶ cells) using the Impel Rat Intranasal Catheter Device (Impel Neuropharma, USA). The catheter tube is inserted into the nasal cavity of the animal through a catheter guide, inserted into the naris of the animal, 5 mm deep, directing the catheter tube to the upper olfactory region. Cells are slowly released through the catheter tube onto the olfactory epithelium, avoiding contact with the IN mucosa. Following i.n. administration, the head of the animal is held in a tilted back position for 1-2 minutes before extraction of catheter from nares to prevent loss of solution from the nares.

(Optional) Neurogenesis Studies:

ASC are localized in the brain, as described for the previous Examples. To determine neurogenesis in the DG, 1 day after administration of ASC, rats in groups 1, 2, 4, and 6 are given 3 administrations of 50 mg/kg BrdU intraperitoneally, spaced apart by 4-hour intervals. After sacrifice, rats are perfused with formaldehyde, and DG specimens are removed from fixed and frozen brains. Neurogenesis in this region is measured by counting the amount of BrdU positive neurons 28 days after ASC administration. For BrdU staining, brain sections of the DG area (multiple sections per rat) are washed with PBS, incubated with 2N HCl at 37° C. for 30 minutes, washed 3 times with PBS, incubated with blocking solution containing 0.5% Triton X-100 and 20% horse serum in PBS for 1 hour, then again washed in PBS. Staining is performed with anti-BrdU and anti-NeuN antibodies, followed by washing and staining with conjugated secondary antibodies. BrdU- and NeuN-positive cells are quantified by microscopic examination.

Additionally, RNA expression levels of miRNA-124, miRNA-451, miRNA-484, NGF, bFGF, GDNF, and/or BDNF are measured in the hippocampus and in the NA, VTA and PFC areas and CSF and serum. mRNA and miRNA levels are tested by quantitative real time PCR (qRT-PCR) (PerfeCTa SYBR Green FastMix, [Quanta BioSciences, Gaithersburg, Montgomery]). Protein levels in serum and CSF are measured by Luminex or ELISA.

TABLE 5
List of primers.

| | Forward primer | Reverse primer |
|---|---|---|
| Gene name | 5′→3′; SEQ No. | 5′→3′; SEQ No. |
| FGF-2 | aattgtgtccatca | aaccagcagccatc |
| | agggatgt; 3 | cat; 4 |
| IGF-1 | gacgtaccaaaatg | tcgataggggctgg |
| | agcgcacc; 5 | gacttc; 6 |
| GDNF family | aaacccatgcctcc | cctgggtagctggg |
| receptor alpha | ggaat; 7 | agtgag; 8 |
| 2 (GFRA2) | | |
| Excitatory | ctggggacctcctc | ccacgggaagcaca |
| amino acid | aagttct; 9 | aatctg; 10 |
| transporter 1 | | |
| (EAAT1) | | |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace alternatives, modifications and variations that fall within the spirit and broad scope of the claims and description. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the invention.

### REFERENCES

### Additional references may be cited in text

Berglind W J et al, A single intra-PFC infusion of BDNF prevents cocaine-induced alterations in extracellular glutamate within the nucleus accumbens. J Neurosci. 2009 Mar. 25; 29(12):3715-9. doi: 10.1523/JNEUROSCI.5457-08.2009.
Betzer O et al, In-vitro Optimization of Nanoparticle-Cell Labeling Protocols for In-vivo Cell Tracking Applications. Sci Rep. 2015 Oct. 28:5:15400. doi: 10. 1038/srep 15400.
Chase L G et al, Development and Characterization of a Clinically Compliant Xeno-Free Culture Medium in Good Manufacturing Practice for Human Multipotent Mesenchymal Stem Cells. Stem Cells Transl Med. 2012 October; 1(10): 750-758.
Clayton A et al, Analysis of antigen presenting cell derived exosomes, based on immuno-magnetic isolation and flow cytometry. J Immunol Methods. 2001; 247(1-2): 163-74.
Crescitelli R et al, Distinct RNA profiles in subpopulations of extracellular vesicles: apoptotic bodies, microvesicles and exosomes. J Extracell Vesicles. 2013 Sep. 12; 2.
Das M et al, Lateral fluid percussion injury of the brain induces CCL20 inflammatory chemokine expression in rats. J Neuroinflammation. 2011 Oct. 31; 8:148. doi: 10.1186/1742-2094-8-148.
Dominici et al. Minimal criteria for defining multipotent mesenchymal stromal cells. The International Society for Cellular Therapy position statement. Cytotherapy. 2006; 8(4):315-7.
Hartl R et al, Blood-brain barrier breakdown occurs early after traumatic brain injury and is not related to white blood cell adherence. Acta Neurochir Suppl. 1997; 70:240-2.
Imamura. Physiological Functions and Underlying Mechanisms of Fibroblast Growth Factor (FGF) Family Members: Recent Findings and Implications for Their Pharmacological Application. Biological and Pharmaceutical Bulletin, Vol. 37(7): 1081-1089 (2014).
Kannagi R et al, Stage-specific embryonic antigens (SSEA-3 and −4) are epitopes of a unique globo-series ganglioside isolated from human teratocarcinoma cells. EMBO J. 1983; 2(12):2355-61.
Kinzebach S and Bieback K. Expansion of Mesenchymal Stem/Stromal cells under xenogenic-free culture conditions. Adv Biochem Eng Biotechnol. 2013; 129:33-57.
Licata S C et al, Repeated stimulation of L-type calcium channels in the rat ventral tegmental area mimics the initiation of behavioral sensitization to cocaine. Psychopharmacology (Berl). 2000 September; 152(1): 110-8.
Liu P1 et al, MRI assessment of cerebral oxygen metabolism in cocaine-addicted individuals: hypoactivity and dose dependence. NMR Biomed. 2014 June; 27(6):726-32. doi: 10. 1002/nbm. 3114.
Mathias R A et al, Isolation of extracellular membranous vesicles for proteomic analysis. Methods Mol Biol. 2009, 528:227-42.
Mimura et al, Growth factor-defined culture medium for human mesenchymal stem cells. Int. J. Dev. Biol. 55: 181-187 (2011).
Nizamutdinov D et al, Overview of Traumatic Brain Injury: An Immunological Context. Brain Sci. 2017 Jan. 23; 7(1). pii: E11. doi: 10.3390/brainsci7010011.
Pomierny-Chamiolo L et al, Cocaine self-administration, extinction training and drug-induced relapse change metabotropic glutamate mGlu5 receptors expression: Evidence from radioligand binding and immunohistochemistry assays. Brain Res. 2017 Jan. 15; 1655:66-76. doi: 10. 1016/j.brainres.2016.11.014.
Rajaraman G et al, Optimization and scale-up culture of human endometrial multipotent mesenchymal stromal cells: potential for clinical application. Tissue Eng Part C Methods. 2013 January; 19(1):80-92.
Vallender E J et al, Transcriptomic profiling of the ventral tegmental area and nucleus accumbens in rhesus macaques following long-term cocaine self-administration. Drug Alcohol Depend. 2017 Jun. 1; 175:9-23. doi: 10.1016/j.drugalcdep.2017.01.030.
Waters R P I et al, Assessment of a proposed “three-criteria” cocaine addiction model for use in reinstatement studies with rats. Psychopharmacology (Berl). 2014 August; 231(16):3197-205. doi: 10.1007/s00213-014-3497-2.
Xiong Y et al, Animal models of traumatic brain injury. Nat Rev Neurosci. 2013 February; 14(2):128-42. doi: 10.1038/nrn3407.

What is claimed is:

1. A method of treating an addiction in a subject in need thereof, comprising: administering to the subject a pharmaceutical composition, comprising placental adherent stromal cells (ASC), thereby treating an addiction.

2. A method of treating an addiction in a subject in need thereof, comprising: administering to the subject a pharmaceutical composition, comprising adherent stromal cells (ASC) not derived from placental tissue, thereby treating an addiction.

3. The method of claim 1, wherein said administering comprises:
a. administering to the subject a first pharmaceutical composition, comprising ASC from a first donor; and
b. administering to said subject, at least 7 days after step a), a second pharmaceutical composition comprising allogeneic ASC from a second donor, wherein said second donor differs from said first donor in at least one allele group of human leukocyte antigen (HLA)-A or human leukocyte antigen (HLA)-B,

4. The method of claim 3, further comprising administering to said subject, at least 7 days after step b), a third pharmaceutical composition comprising allogeneic ASC of a third donor, wherein said third donor differs from both said first donor and said second donor in at least one allele group of HLA-A or HLA-B.

5. The method of claim 1, wherein said ASC secrete a factor selected from BDNF (brain derived neurotrophic factor), GDNF (glial cell line-derived neurotrophic factor), bFGF (basic fibroblast growth factor), NGF (beta-nerve growth factor), VEGF (vascular endothelial growth factor), and HGF (hepatocyte growth factor), and LIF (Leukemia inhibitory factor).

6. The method of claim 1, wherein said ASC have been incubated in a 3D culture apparatus.

7. The method of claim 6, further comprising the subsequent step of harvesting said ASC by removing said ASC from said 3D culture apparatus.

8. The method of claim 6, wherein said ASC have been incubated in a 2D adherent-cell culture apparatus, prior to said incubation in a 3D culture apparatus.

9. The method of claim 1, wherein said 3D culture apparatus comprises a bioreactor.

10-12. (canceled)

13. The method of claim 1, wherein said 3D culture apparatus comprises microcarriers.

14. (canceled)

15. The method of claim 1, wherein said addiction is an addiction to a psychostimulant.

16-18. (canceled)

19. The method of claim 1, wherein said ASC express a marker selected from the group consisting of CD73, CD90, CD29 and CD105.

20. The method of claim 19, wherein said ASC do not express a marker selected from the group consisting of CD3, CD4, CD11b, CD14, CD19, and CD34.

21. The method of claim 19, wherein said ASC do not express a marker selected from the group consisting of CD3, CD4, CD34, CD39, and CD106.

22. The method of claim 21, wherein less than 50% of said ASC express CD200.

23. The method of claim 21, wherein more than 50% of said ASC express CD200.

24-26. (canceled)

27. The method of claim 2, wherein said ASC originate from adipose tissue.

28. The method of claim 2, wherein said ASC originate from bone marrow.

29-31. (canceled)

32. The method of claim 1, wherein the cells are administered intranasally.

33. The method of claim 1, wherein the cells are administered intra-cerebro-ventricularly.


SEQUENCE LISTING (10 sequences)

SEQ ID NO: 1 - DNA, 20 bp, Artificial Sequence
  primer
gcggcagataaaaagactgc

SEQ ID NO: 2 - DNA, 20 bp, Artificial Sequence
  primer
gccagccaattctctttttg

SEQ ID NO: 3 - DNA, 22 bp, Artificial Sequence
  primer
agttgtgtccatcaagggatgt

SEQ ID NO: 4 - DNA, 17 bp, Artificial Sequence
  primer
agccagcagccgtccat

SEQ ID NO: 5 - DNA, 22 bp, Artificial Sequence
  primer
gacgtaccaaaatgagcgcacc

SEQ ID NO: 6 - DNA, 20 bp, Artificial Sequence
  primer
tcgataggggctgggacttc

SEQ ID NO: 7 - DNA, 19 bp, Artificial Sequence
  primer
aaacccatgcctccggaat

SEQ ID NO: 8 - DNA, 20 bp, Artificial Sequence
  primer
cctgggtagctgggagtgag

SEQ ID NO: 9 - DNA, 21 bp, Artificial Sequence
  primer
ctggggacctcctcaagttct

SEQ ID NO: 10 - DNA, 20 bp, Artificial Sequence
  primer
ccacgggaagcacaaatctg